SEQUENCE LISTING (4 sequences)

SEQ ID NO: 1 - protein, 448 aa, Homo sapiens
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Homo sapiens [sequence omitted]

SEQ ID NO: 3 - protein, 448 aa, Homo sapiens
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 4 - protein, 214 aa, Homo sapiens [sequence omitted]

(19) United States
(12) Patent Application Publication
(10) Pub. No.: US 2016/0176921 A1
(43) Pub. Date: Jun. 23, 2016

(54) METHODS OF PURIFYING RECOMBINANT PROTEINS

(71) Applicant: Alexion Pharmaceuticals, Inc., Cheshire, CT (US)

(72) Inventors: Saravanamoorthy Rajendran, Cheshire, CT (US); Luca Di Noto, Cheshire, CT (US)

(21) Appl. No.: 14/977,869

(22) Filed: Dec. 22, 2015

Related U.S. Application Data
(60) Provisional application No. 62/095,281, filed on Dec. 22, 2014.

(51) Int. Cl. C07K 1/36; C07K 1/18; C07K 1/16; B01D 15/32; C07K 1/20; C07K 16/18; B01D 15/38; B01D 15/36; C07K 1/22; C07K 1/34

(52) U.S. Cl. CPC C07K 1/36; C07K 1/22; C07K 1/18; C07K 1/165; C07K 1/34; C07K 1/20; C07K 16/18; B01D 15/3804; B01D 15/363; B01D 15/362; B01D 15/327; C07K 2317/24; C07K 2317/76

(57) ABSTRACT

Provided herein are methods of purifying a recombinant protein and methods of manufacturing a recombinant protein product that include capturing a recombinant protein from a solution including the recombinant protein, following capturing, performing one or more unit operations on the solution, and after capturing and performing one or more unit operations, flowing the recombinant protein through a depth filter to provide a filtrate that includes purified recombinant protein and is substantially free of soluble protein aggregates.

### CROSS-REFERENCE TO RELATED APPLICATIONS

This application claims priority to U.S. provisional patent application 62/095,281, filed Dec. 22, 2014, the entire contents of which are incorporated herein by reference.

### TECHNICAL FIELD

This invention relates generally to methods of purifying recombinant proteins and methods of manufacturing recombinant protein products.

### BACKGROUND

Recombinant proteins, such as monoclonal antibodies (mAb), are an important and valuable class of therapeutic products for treating diseases, such as paroxysmal nocturnal hemoglobinuria (PNH) and atypical hemolytic uremic syndrome (aHUS). Mammalian cells including a nucleic acid that encodes a recombinant protein are often used to produce the recombinant protein. The recombinant protein is then purified from the mammalian cell culture using a process that can include passing a fluid that includes the recombinant protein through one or more filters. These purification processes may exhibit slow flow rates (low filter throughput) and/or fouling due to plugging of one or more filters in the process with soluble protein aggregates that can include protein dimer, trimers, or higher protein polymers. The contaminants/impurities and/or fouling of one or more filters in a purification or manufacturing process can result in recombinant protein loss, implementation of additional purification steps, and/or can negatively impact the safety of the resulting recombinant protein product.

### SUMMARY

The present disclosure is based, at least in part, on the discovery that a method of purifying a recombinant protein, such as a monoclonal antibody (such as eculizumab or Alexion 1210), that includes a clarification step, a capture step, one or more unit operations involving various types of column chromatography, viral inactivation, and viral filtration, can benefit from strategic placement of a depth filtration step to remove protein aggregates and host cell proteins.

Provided herein are methods of purifying a recombinant protein that include: (a) capturing a recombinant protein from a solution including the recombinant protein; (b) following capturing, performing one or more unit operations on the solution; and (c) following steps (a) and (b), flowing the recombinant protein through a depth filter to provide a filtrate that includes purified recombinant protein and is substantially free of soluble protein aggregates. In some embodiments of any of the methods described herein, following flowing the recombinant protein through the depth filter, the filtrate is flowed through one or more additional depth filters or a viral filter. Some embodiments of any of the methods described herein further include prior to step (a): performing one or more unit operations selected from the group of: ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution including the recombinant protein.

Also provided are methods of manufacturing a recombinant protein product that include: (a) capturing a recombinant protein from a clarified liquid culture medium including the recombinant protein; (b) following capturing, performing one or more unit operations on the solution; (c) following steps (a) and (b), flowing the recombinant protein through a depth filter to provide a filtrate that includes purified recombinant protein and is substantially free of soluble protein aggregates; and (d) performing one or more unit operations on the purified recombinant protein, thereby producing the recombinant protein product. In some embodiments of these methods, the solution including the recombinant protein in step (a) is a clarified liquid culture medium or a buffered solution including the recombinant protein. In some embodiments of these methods, the one or more unit operations in step (d) is selected from the group of: purifying the recombinant protein, polishing the recombinant protein, inactivating viruses, removing viruses by filtration, adjusting one or both of the pH and ionic concentration of a solution comprising the purified recombinant protein, and passing the fluid through an additional depth filter. In some embodiments of these methods, the one or more unit operations in step (d) includes or is removing viruses by filtration. In some embodiments of these methods, step (d) includes performing the unit operations of purifying the recombinant protein and performing viral filtration. In some embodiments of these methods, step (d) includes performing the unit operations of polishing the recombinant protein and performing viral filtration. In some embodiments of these methods, step (d) includes performing the unit operations of purifying the recombinant protein (e.g., through cation exchange chromatography), polishing the recombinant protein (e.g., through anion exchange chromatography), and performing viral filtration. In some embodiments of these methods, step (d) includes performing the unit operations of ultrafiltration/diafiltration, purifying the recombinant protein (e.g., through cation exchange chromatography), polishing the recombinant protein (e.g., through anion exchange chromatography), and performing viral filtration.

In some embodiments of any of the methods described herein, the capturing is performed using an affinity chromatography resin, an anionic exchange chromatography resin, a cationic exchange chromatography resin, a mixed-mode chromatography resin, a molecular sieve chromatography resin, or a hydrophobic interaction chromatography resin.

In some embodiments of any of the methods described herein, the affinity chromatography resin utilizes a capture mechanism selected from the group of: a protein A-binding capture mechanism, an antibody- or antibody fragment-binding capture mechanism, a substrate-binding capture mechanism, and a cofactor-binding capture mechanism.

In some embodiments of any of the methods described herein, the one or more unit operations in step (b) is selected from the group of: ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein. In some embodiments of any of the methods described herein, the polishing is performed using hydrophobic interaction chromatography or ion-exchange chromatography.

In some embodiments of any of the methods described herein, the one or more unit operations in step (b) is polishing using hydrophobic interaction chromatography and ultrafiltration/diafiltration to concentrate the recombinant protein in a solution. In some embodiments of any of the methods described herein, the one or more unit operations in step (b) is viral inactivation and adjustment of one or both pH and ionic strength of a solution including the recombinant protein.

In some embodiments of any of the methods described herein, the recombinant protein is flowed through the depth filter in a solution having a pH of between about 4.0 to about 7.5 (e.g., between about 5.5 to about 7.5, or between about 6.5 to about 7.5). In some embodiments of any of the methods described herein, the recombinant protein is flowed through the depth filter and both protein aggregates (e.g., soluble protein aggregates) and host cell protein (HCP) are reduced by at least 50% (e.g., at least 55%, at least 60%, at least 65%, at least 70%, at least 75%, at least 80%, at least 85%, or at least 90%). In some embodiments of any of the methods described herein, the recombinant protein is flowed through the depth filter at a flow rate of between about 25 L/m²/h to about 400 L/m²/h (e.g., between about 70 L/m²/h to about 150 L/m²/h). In some embodiments of any of the methods described herein, the depth filter has a membrane surface area of between about 10 cm² to about 32000 cm² (e.g., between about 10 cm² to about 1020 cm²). In some embodiments of any of the methods described herein, the depth filter includes a filtration medium that is positively charged. In some embodiments of any of the methods described herein, the depth filter includes a filtration medium that comprises silica.

In some embodiments of any of the methods described herein, the filtrate including purified recombinant protein in step (c) further includes a reduced level of host cell protein as compared to a level of host cell protein in the recombinant protein that is flowed through the depth filter in step (c).

In some embodiments of any of the methods described herein, the recombinant protein is an antibody (e.g., a human or humanized antibody). In some embodiments of any of the methods described herein, the antibody specifically binds to human complement protein C5 (e.g., eculizumab or Alexion 1210). In some embodiments of any of the methods described herein, the antibody consists of a heavy chain comprising SEQ ID NO: 1 and a light chain comprising SEQ ID NO: 2. In some embodiments of any of the methods described herein,

the antibody consists of a heavy chain consisting of SEQ ID NO: 1 and a light chain consisting of SEQ ID NO: 2.

As used herein, the word “a” before a noun represents one or more of the particular noun. For example, the phrase “a depth filter” represents “one or more depth filters.”

The term “mammalian cell” means any cell from or derived from any mammal (e.g., a human, a hamster, a mouse, a green monkey, a rat, a pig, a cow, or a rabbit). For example, a mammalian cell can be an immortalized cell. The mammalian cell can be a differentiated or undifferentiated cell. Non-limiting examples of mammalian cells are described herein. Additional examples of mammalian cells are known in the art.

The term “substantially free” means a composition (e.g., a filtrate) that is at least or about 90% free, such as at least or about 95%, 96%, 97%, 98%, or at least or about 99% free, or about 100% free of a specified substance, such as soluble protein aggregates or host cell proteins.

The term “culturing” or “cell culturing” means maintenance or proliferation of a mammalian cell under a controlled set of physical conditions.

The term “culture of mammalian cells” means a culture medium (such as a liquid culture medium) including a plurality of mammalian cells that is maintained or proliferated under a controlled set of physical conditions.

The term “liquid culture medium” means a fluid that includes sufficient nutrients to allow a cell (such as a mammalian cell) to grow or proliferate in vitro. A liquid culture medium can include, for example, one or more of: amino acids (such as 20 amino acids), a purine (such as hypoxanthine), a pyrimidine (such as thymidine), choline, inositol, thiamine, folic acid, biotin, calcium, niacinamide, pyridoxine, riboflavin, thymidine, cyanocobalamin, pyruvate, lipoic acid, magnesium, glucose, sodium, potassium, iron, copper, zinc, and sodium bicarbonate. In some embodiments, a liquid culture medium can include serum from a mammal. In some embodiments, a liquid culture medium does not include serum or another extract from a mammal (a defined liquid culture medium). A liquid culture medium can also include trace metals, a mammalian growth hormone, and/or a mammalian growth factor. An example of liquid culture medium is minimal medium (such as a medium including only inorganic salts, a carbon source, and water). Non-limiting examples of liquid culture medium are described herein. Additional examples of liquid culture medium are known in the art and are commercially available. A liquid culture medium can include any density of mammalian cells. For example, as used herein, a volume of liquid culture medium removed from a vessel (such as a bioreactor) can be substantially free of mammalian cells.

The term “immunoglobulin” means a polypeptide including an amino acid sequence of at least 10 amino acids (such as at least 15, 20, 30, 40, 50, 60, 70, 80, 90, or 100 amino acids) of an immunoglobulin protein (such as a variable domain sequence, a framework sequence, or a constant domain sequence of a heavy or light chain immunoglobulin). The immunoglobulin may be an isolated antibody (such as an IgG, IgE, IgD, IgA, or IgM). The immunoglobulin may be any subclass of IgG, such as IgG1, IgG2, IgG3, or IgG4, or the chimeric IgG2/4 as found in eculizumab or Alexion 1210. The immunoglobulin may be an antibody fragment, such as a Fab fragment, a F(ab′)₂ fragment, or an scFv fragment. The immunoglobulin may be a bi-specific antibody or a tri-specific antibody, or a dimer, trimer, or multimer antibody, or a diabody, an AFFIBODY®, or a NANOBODY®. The immunoglobulin can be an engineered protein including at least one immunoglobulin domain (such as a fusion protein including a Fc domain). The immunoglobulin can be an engineered protein having four antibody binding domains such as DVD-Ig and CODV-Ig. See US2007/0071675 and WO2012/135345. Non-limiting examples of immunoglobulins are described herein and additional examples of immunoglobulins are known in the art.

The term “protein fragment” or “polypeptide fragment” means a portion of a polypeptide sequence that is at least or about 5 amino acids, at least or about 6 amino acids, at least or about 7 amino acids, at least or about 8 amino acids, at least or about 9 amino acids, at least or about 10 amino acids, at least or about 11 amino acids, at least or about 12 amino acids, at least or about 13 amino acids, at least or about 14 amino acids, at least or about 15 amino acids, at least or about 16 amino acids, at least or about 17 amino acids, at least or about 18 amino acids, at least or about 19 amino acids, or at least or about 20 amino acids in length, or more than 20 amino acids in length.

The term “capturing” means a step performed to partially purify or isolate (such as at least or about 10%, 15%, 20%, 25%, 30%, 40%, 45%, 50%, 55%, 60%, 65%, 70%, 75%, 80%, 85%, 90%, or 95%, or at least or about 99% pure by weight), concentrate, and/or stabilize a recombinant protein from one or more other components present in a solution including the recombinant protein. Other components may include buffers, salts, DNA, RNA, host cell proteins, and aggregates of the desired recombinant protein present in or secreted from a mammalian cell. Capturing can be performed using a chromatography resin that binds a recombinant protein through the use of a specific recognition and binding interaction, such as with protein A chromatography and antibody capture. Non-limiting methods for capturing a recombinant protein from a solution including the recombinant protein or a clarified liquid culture medium are described herein and others are known in the art. A recombinant protein can be captured from a liquid culture medium using at least one chromatography column (such as any of the chromatography columns described herein, such as a chromatography column packed with an affinity chromatography resin, an anionic exchange chromatography resin, a cationic exchange chromatography resin, a mixed-mode chromatography resin, a molecular sieve chromatography resin, or a hydrophobic interaction chromatography resin). Capturing can be performed using a chromatography resin that utilizes a protein A-binding capture mechanism, an antibody- or antibody fragment-binding capture mechanism, a substrate binding capture mechanism, or a cofactor-binding capture mechanism.

The term “purifying” means a method or step performed to isolate a recombinant protein from one or more other impurities or components present in a fluid including a recombinant protein. The components being separated include liquid culture medium proteins, host cell proteins, aggregates of the desired recombinant protein, DNA, RNA, other proteins, endotoxins, and viruses present in or secreted from a mammalian cell. For example, a purifying step can be performed before or after an initial capturing step and/or before or after a step of flowing a recombinant protein through a depth filter. A purifying step can be performed using a resin, membrane, or any other solid support that binds either a recombinant protein or contaminants (such as through the use of affinity chromatography, hydrophobic interaction chromatography, anion or cation exchange chromatography, mixed-mode chromatography resin, or molecular sieve chromatography). A recombinant protein can be purified from a solution including the recombinant protein using at least one chromatography column and/or chromatographic membrane (such as any of the chromatography columns described herein).

The term “polishing” is a term of art and means a step performed to remove remaining trace or small amounts of contaminants or impurities from a fluid including a manufactured recombinant protein that is close to a final desired purity. For example, polishing can be performed by passing a solution including the recombinant protein through a chromatographic column(s) or membrane absorber(s) that selectively binds to either the target recombinant protein or small amounts of remaining contaminants or impurities present in the solution including the recombinant protein. In such an example, the eluate/filtrate of the chromatographic column(s) or membrane absorber(s) includes the recombinant protein. As described herein, one or more unit operations of polishing can be performed prior to flowing a solution comprising the recombinant protein through a depth filter.

The terms “eluate” and “filtrate” are terms of art and mean a fluid that is emitted from a depth filter, chromatography column, or chromatographic membrane that includes a detectable amount of a recombinant protein.

The term “filtering” means the removal of at least part of (such as at least 90%, 95%, 96%, 97%, 98%, or 99%) undesired biological contaminants (such as a mammalian cell, bacteria, yeast cells, viruses, mycobacteria, or mycoplasma), impurities (such as soluble protein aggregates, host cell proteins, host cell DNA, and other chemicals used in a method for purifying a recombinant protein or a method of manufacturing a recombinant protein product), and/or particulate matter (such as precipitated proteins) from a liquid (such as a liquid culture medium or fluid present in any of the systems or processes described herein).

The term “secreted protein” or “secreted recombinant protein” means a protein (such as a recombinant protein) that originally included at least one secretion signal sequence when it is translated within a mammalian cell, and through, at least in part, enzymatic cleavage of the secretion signal sequence in the mammalian cell, is secreted at least partially into the extracellular space (such as a liquid culture medium). Skilled practitioners will appreciate that a “secreted” protein need not dissociate entirely from the cell to be considered a secreted protein.

The term “clarified liquid culture medium” means a liquid culture medium obtained from a mammalian, bacterial, or yeast cell culture that is substantially free (such as at least 90%, 92%, 94%, 96%, 98%, or 99% free) of mammalian, bacterial, or yeast cells. A clarified liquid culture medium can be prepared, for example, by filtering a cell culture (such as alternating tangential filtration or tangential flow filtration), by centrifuging a cell culture and collecting the supernatant, or by allowing the cells in the cell culture settle and obtaining a fluid that is substantially free of cells. The cells can also be separated from the medium by the use of a cell separation device, such as the ATF system from Refine Technology.

Purification of manufactured recombinant proteins usually requires in series performance of multiple independent purification operations or steps. The term “unit operation” is a term of art and means a discreet step or mini-process performed in a larger general process for purifying a recombinant protein or a method of manufacturing a recombinant protein product (such as a method of manufacturing a recombinant protein product from a clarified liquid culture medium). For example, a unit of operation can be a step of capturing the recombinant protein, ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein.

The term “filter medium” is a term of art and means a material that captures contaminants and/or impurities within its structure. For example, a filter medium can include multiple layers, a single layer, multiple layers or membranes, a gel, a matrix, or a packed chromatography resin. A filter medium can be comprised of silica (such as positively charged silica). A filter medium can be positively or negatively charged.

The term “depth filter” is a term of art and means a filter that includes a porous filtration medium that captures contaminants and/or impurities (such as any of the contaminants and/or impurities described herein) within its 3-dimensional structure and not merely on the surface. Depth filters are characterized in that they retain the contaminants or impurities within the filter and can retain a relatively large quantity before becoming clogged. Depth filter construction may comprise multiple layers, multiple membranes, a single layer, or a resin material. Non-limiting examples of depth filters include CUNO® Zeta PLUS® Delipid filters (3M, St. Paul, Minn.), CUNO® Emphaze AEX filters (3M, St. Paul, Minn.), CUNO® 90ZA08A filters (3M, St. Paul, Minn.), CUNO® DELI08A Delipid filters (3M, St. Paul, Minn.), Millipore XOHC filters (EMD Millipore, Billerica, Mass.), MILLISTAK® pads (EMD Millipore, Billerica, Mass.).

The term “soluble protein aggregates” is a term of art and means complexes of two or more proteins (such as recombinant proteins) that are soluble in a solution. Such complexes can form through hydrophobic and/or ionic interactions between individual recombinant protein molecules or fragments thereof.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic showing different recombinant Fc-fusion protein purification methods tested and the amount of soluble protein aggregates present in different steps of the tested methods. This figures does not show an ultrafiltration/diafiltration step that occurs prior to the protein A chromatography in each method tested.

FIG. 2 is a graph showing the flux decay as a function of viral filter throughput achieved in different processes used to purify a recombinant Fc fusion protein that include prior to the viral filtration step: clarification of a culture medium, ultrafiltration/diafiltration, protein A capturing, hydrophobic interaction chromatography (e.g., polishing), concentration to a recombinant protein concentration of 5 mg/mL, and pre-filtration (blue diamonds and red squares); clarification of a culture medium, ultrafiltration/diafiltration, protein A capturing, hydrophobic interaction chromatography (e.g., polishing), concentration to a recombinant protein concentration of 7.5 mg/mL, and pre-filtration (green triangles and lavender Xs); clarification of a culture medium, ultrafiltration/diafiltration, protein A capturing, hydrophobic interaction chromatography (e.g., polishing), concentration to a recombinant protein concentration of 10 mg/mL, and pre-filtration (green asterisks and orange circles); or clarification of a culture medium, ultrafiltration/diafiltration, protein A capturing, hydrophobic interaction chromatography (e.g., polishing, ultrafiltration/diafiltration, pre-filtration, and Delipid Virosart depth filtration, where the eluate of the depth filter has a recombinant protein concentration of 4.6 mg/mL (green plus signs and peach dashes).

FIG. 3 is a schematic diagram showing the unit operations following protein A chromatography (capturing) in three different tested methods of purifying a recombinant Fc fusion protein. Each test method further comprises prior to protein A chromatography (capturing) the steps of clarification of a culture medium, ultrafiltration/diafiltration, and viral inactivation.

FIG. 4 is a graph showing the flux decay as a function of viral filter throughput achieved in different processed used to purify a recombinant Fc fusion protein that include prior to the viral filtration step: clarification of a culture medium, ultrafiltration/diafiltration, viral inactivation, protein A chromatography (capturing), hydrophobic interaction chromatography (e.g., polishing), and ultrafiltration/diafiltration (Experiment 1; run 1 and run 2); clarification of a culture medium, ultrafiltration/diafiltration, viral inactivation, protein A chromatography (capturing), hydrophobic interaction chromatography (e.g., polishing), ultrafiltration/diafiltration, and depth filtration (Experiment 2; runs 1 and run 2); or clarification of a culture medium, ultrafiltration/diafiltration, viral inactivation, protein A chromatography (capturing), hydrophobic interaction chromatography (e.g., polishing), depth filtration, and ultrafiltration/diafiltration (Experiment 3; runs 1 and 2).

FIG. 5 is a diagram of the three tested processes in Example 4 (Schematics 1 to 3) and the process yields.

FIG. 6 is a diagram of the three tested processes in Example 4 (Schematics 1 to 3) and the percentage of soluble protein aggregates, the percentage of eculizumab monomers, and the percentage of eculizumab fragments at each step in the three tested processes.

FIG. 7 is a set of three chromatograms from the Capto Adhere ImpRes chromatography steps performed in each of the tested processes in Example 4 (Schematics 1 to 3).

FIG. 8 is a set of three isoelectric focusing capillary electrophoresis spectra from the Capto Adhere ImpRes chromatography steps performed in each of the tested processes in Example 4 (Schematics 1 to 3).

FIG. 9 is a graph showing the flux decay and the filter inlet pressure as a function of the volumetric throughput of the viral filters used in the tested Schematic 1 process in Example 4.

FIG. 10 is a graph showing the flux decay and the filter inlet pressure as a function of the volumetric throughput of the viral filters used in the tested Schematic 2 process in Example 4.

FIG. 11 is a graph showing the flux decay and the filter inlet pressure as a function of the volumetric throughput of the viral filters used in the tested Schematic 3 process in Example 4.

FIG. 12 is a graph showing the turbidity and compared to the pH of protein A chromatography pooled material that has been adjusted to a conductivity of 0.95 mS/cm, 2.07 mS/cm, 5.01 mS/cm, 8.95 mS/cm, or 15.55 mS/cm via ultrafiltration/diafiltration (UF/DF1 step).

FIG. 13 is a schematic showing the different processes tested in Example 6.

FIG. 14 is a schematic showing the process steps tested in Example 7.

FIG. 15 is a graph showing the percentage recovery of a biparatopic antibody of Alexion 1210 and percentage of soluble protein aggregates in Delipid depth filter filtrate at different loads (g/m²).

### DETAILED DESCRIPTION

During manufacturing of a recombinant protein, such as a monoclonal antibody such as eculizumab or Alexion 1210, soluble protein aggregates are known to form in solution. The cell culture phase of a typical recombinant protein production process often includes secretion of the recombinant protein from the cell into a liquid culture medium, which includes cells, liquid culture medium ingredients, nutrients for the cells, host-cell proteins (including proteases), dissolved oxygen, and other compounds. The cell culture (including the liquid culture medium) is typically held at near neutral pH at temperatures above 30° C. for several days. Once a sufficient amount of recombinant protein has been expressed, the liquid culture medium is typically clarified, harvested, and the recombinant protein is purified by one or more unit operations (such as multiple chromatography steps). For example, purification of recombinant proteins can include incubating a solution including the recombinant protein at an acidic pH in order to achieve viral inactivation. Purification methods can include a step performed under conditions of high conductivity, such as cation exchange chromatography, and/or a step performed at high pH, such as anion exchange chromatography. The methods can include a step of ultrafiltration/diafiltration. Throughout the purification methods, a solution including a recombinant protein is pumped, stirred, filtered, and exposed to a variety of materials including stainless steel, glass, and plastic. Exposure to these conditions of pH, ionic strength, temperature, concentration, shear forces, and other processing conditions can result in formation of recombinant protein aggregates. Methods for purifying a recombinant protein can include using one or more filters. These filters can become clogged with recombinant protein aggregates (such as soluble protein aggregates that can include soluble recombinant protein aggregates), and as a result, the throughput of the filter(s) can become reduced and/or the filter(s) can become fouled. In addition, the presence of protein aggregates (such as soluble recombinant protein aggregates) can decrease the yield of purified recombinant protein and/or a decrease the safety (such as by causing a change in characteristics that increase the immunogenicity of the product) of the resulting purified recombinant protein product.

The methods provided herein provide for one or more of the following benefits (in any combination): a reduction in the levels of soluble protein aggregates (such as soluble protein aggregates that include soluble recombinant protein aggregates) in a method of purifying a recombinant protein or a method of manufacturing a recombinant protein product or in a system used to perform the same, an increase in the throughput of one or more filters (such as a virus filter) used in a method of purifying a recombinant protein or a method of manufacturing a recombinant protein product, an improvement of the safety profile of a purified recombinant protein or a recombinant protein product, a reduction in the total number of unit operations required to purify a recombinant protein or to manufacture a recombinant protein product, a decrease in the cost of a method of purifying a recombinant protein or a method of manufacturing a recombinant protein product, a shorter period of time to obtain a purified recombinant protein or a recombinant protein product (such as when starting from a culture medium), a decreased level of immunogenicity in a purified recombinant protein or recombinant protein product following administration to a subject (such as a human subject) (as compared to a recombinant protein purified or manufactured by a method that does not include a step of flowing the recombinant protein through a depth filter after a step of capturing the recombinant protein), a reduced risk of filter fouling or contamination in a method of purifying a recombinant protein and a method of manufacturing a recombinant protein product or in a system used to perform the same, and a reduced level of host cell protein in the purified recombinant protein (as compared to a method that does not include a step of flowing the recombinant protein through a depth filter after a step of capturing the recombinant protein (and optionally further after one or more additional unit operations) or a system used to perform the same).

Provided herein are methods of purifying a recombinant protein and methods of manufacturing a recombinant protein product. The methods can include, for example, (a) capturing a recombinant protein from a solution including the recombinant protein, e.g., a clarified liquid culture medium or a buffered solution including the recombinant protein; (b) following capturing, performing one or more unit operations on the solution; and following steps (a) and (b), flowing the recombinant protein through a depth filter to provide a filtrate that includes purified recombinant protein and is substantially free of soluble protein aggregates; and optionally, further (d) performing one or more unit operations on the purified recombinant protein. Some embodiments further include, for example, performing at least one (such as two, three, or four) unit operation before the capturing step (e.g., selected from the group of clarifying a culture medium, ultrafiltration/diafiltration to concentrate the recombinant protein in a solution, viral inactivation, and adjusting one or both of the pH and ionic concentration of a solution including the recombinant protein). In some embodiments, step (b) includes performing one or more (such as two, three, or four) unit operations on the solution, e.g., selected from the group of ultrafiltration/diafiltration to concentrate the recombinant protein in a solution, purifying the recombinant protein, polishing the recombinant protein, inactivating viruses, removing viruses by filtration, and adjusting one or both of the pH and ionic concentration of the solution comprising the recombinant protein. In some embodiments, step (b) includes performing viral inactivation and adjusting one or both of the pH and ionic concentration of a solution including the recombinant protein. Some embodiments further include performing one or more (two, three, or four) unit operations after the step of flowing the recombinant protein through a depth filter (e.g., one or more unit operations selected from the group of purifying the recombinant protein, polishing the recombinant protein, inactivating viruses, removing viruses by filtration, adjusting one or both of the pH and ionic concentration of a solution comprising the purified recombinant protein, or passing the fluid through an additional depth filter). In some embodiments, step (d) includes performing viral filtration immediately following the step of flowing the recombinant protein through a depth filter (step (c)). In some embodiments, step (d) includes performing the unit operations of purifying the recombinant protein and performing viral filtration. In some embodiments, step (d) includes performing the unit operations of polishing the recombinant protein and performing viral filtration. In some embodiments, step (d) includes performing the unit operations of purifying the recombinant protein (e.g., through cation exchange chromatography), polishing the recombinant protein (e.g., through anion exchange chromatography), and performing viral filtration. In some embodiments, step (d) includes performing the unit operations of ultrafiltration/diafiltration, purifying the recombinant protein (e.g., through cation exchange chromatography), polishing the recombinant protein (e.g., through anion exchange chromatography), and performing viral filtration.

The methods provided herein can result in a purified recombinant protein that is at least or about 95%, 96%, 97%, 98%, 98.2%, 98.4%, 98.6%, 98.8%, 99.0%, 99.1%, 99.2%, 99.3%, 99.4%, 99.5%, 99.6%, 99.7%, 99.8%, or 99.9% free of soluble protein aggregates or includes no detectable soluble protein aggregates. The methods provided herein can also provide for a reduction, such as up to a 5% reduction, up to a 10% reduction, up to a 15% reduction, up to a 20% reduction, up to a 25% reduction, up to a 30% reduction, up to a 35% reduction, up to a 40% reduction, up to a 45% reduction, up to a 50% reduction, up to a 55% reduction, up to a 60% reduction, up to a 65% reduction, up to a 70% reduction, up to a 75% reduction, up to a 80% reduction, up to an 85% reduction, up to a 90% reduction, up to a 95% reduction, or up to a 99% reduction in host cell protein present in a purified recombinant protein (as compared to a purified recombinant protein produced by a method that does not include a step of flowing the recombinant protein through a depth filter after a step of capturing the recombinant protein (and optionally further after one or more additional unit operations)). Methods for determining the level of host cell protein are well known in the art. For example, kits for detecting the level of host cell protein are commercially available from Cygnus Technologies (Southport, N.C.), ArrayBridge (St. Louis, Mo.), Cisbio (Bedford, Mass.), and Lonza (Basel, Switzerland).

In some embodiments of any of the methods described herein, the filtrate produced in step (c) can include a reduced level (e.g., up to a 5% reduction, up to a 10% reduction, up to a 15% reduction, up to a 20% reduction, up to a 25% reduction, up to a 30% reduction, up to a 35% reduction, up to a 40% reduction, up to a 45% reduction, up to a 50% reduction, up to a 55% reduction, up to a 60% reduction, up to a 65% reduction, up to a 70% reduction, up to a 75% reduction, up to an 80% reduction, up to an 85% reduction, up to a 90% reduction, up to a 95% reduction, or up to a 99% reduction) of host cell protein as compared to a level of host cell protein in the recombinant protein that is flowed through the depth filter (e.g., the level of host cell protein in the recombinant protein flowed or fed into the depth filter in step (c) to generate the filtrate).

In some embodiments of any of the methods described herein, the filtrate produced in step (c) can include a reduced level (e.g., up to 5% reduction, up to 10% reduction, up to a 15% reduction, up to a 20% reduction, up to a 30% reduction, up to a 35% reduction, up to a 40% reduction, up to a 45% reduction, up to a 50% reduction, up to a 55% reduction, up to a 60% reduction, up to a 60% reduction, up to a 70% reduction, up to a 75% reduction, up to a 80% reduction, up to a 85% reduction, up to a 90% reduction, up to a 95% reduction, or up to a 99% reduction) in both the level of soluble protein aggregates and the level of host cell protein (e.g., as compared to a level of soluble protein aggregates and the level of host cell protein in the recombinant protein that is flowed through the depth filter, e.g., the level of soluble protein aggregates and the level of host cell protein in the recombinant protein flowed or fed into the depth filter in step c to generate the filtrate).

In some embodiments of any of the methods described herein, the recombinant protein is flowed through the depth filter and one or both of the level of protein aggregates and the level of host cell protein is/are reduced by at least 10%, at least 15%, at least 20%, at least 25%, at least 30%, at least 35%, at least 40%, at least 45%, at least 50%, at least 55%, at least 60%, at least 65% at least 70%, at least 75%, at least 80%, at least 85%, at least 90%, at least 95%, or at least 99% (e.g., as compared to a level of soluble protein aggregates and/or the level of host cell protein in the recombinant protein that is flowed through the depth filter, e.g., the level of soluble protein aggregates and the level of host cell protein in the recombinant protein flowed or fed into the depth filter in step c to generate the filtrate).

The methods provided herein can also result in a purified recombinant protein that has a decreased level of immunogenicity when administered to a subject (such as a human subject) as compared to a recombinant protein purified by a method that does not include a step of flowing the recombinant protein through a depth filter after a step of capturing the recombinant protein (and optionally also after performing one or more unit operations).

The methods provided herein can further provide for a reduced risk of filter fouling or contamination in a method of purifying a recombinant protein and a method of manufacturing a recombinant protein product or in a system used to perform the same (as compared to a method that does not include a step of flowing the recombinant protein through a depth filter after a step of capturing the recombinant protein (and optionally also after performing one or more unit operations) or a system used to perform the same).

### Recombinant Proteins

Non-limiting examples of recombinant proteins that can be produced by the methods provided herein include immunoglobulins (including light and heavy chain immunoglobulins, antibodies (such as eculizumab or Alexion 1210), or antibody fragments (such as any of the antibody fragments described herein), enzymes, proteins (e.g., human erythropoietin, tumor necrosis factor (TNF), or an interferon alpha or beta), or immunogenic or antigenic proteins or protein fragments (such as proteins for use in a vaccine). The recombinant protein can be an engineered antigen-binding polypeptide that includes at least one multifunctional recombinant protein scaffold (such as the recombinant antigen-binding proteins described in U.S. Patent Application Publication No. 2012/0164066. Non-limiting examples of recombinant proteins that are antibodies include: eculizumab, Alexion 1210, panitumumab, omalizumab, abagovomab, abciximab, actoxumab, adalimumab, adecatumumab, afelimomab, afutuzumab, alacizumab, alacizumab, alemtuzumab, alirocumab, altumomab, amatuximab, amatuximab, anatumomab, anrukinzumab, apolizumab, arcitumomab, atinumab, tocilizumab, basilizimab, bectumomab, belimumab, bevacizumab, besilesomab, bezlotoxumab, biciromab, canakinumab, certolizumab, cetuximab, cixutumumab, daclizumab, denosumab, densumab, edrecolomab, efalizumab, efungumab, epratuzumab, ertumaxomab, etaracizumab, figitumumab, golimumab, ibritumomab tiuxetan, igovomab, imgatuzumab, infliximab, inolimomab, inotuzumab, labetuzumab, lebrikizumab, moxetumomab, natalizumab, obinutuzumab, oregovomab, palivizumab, panitumumab, pertuzumab, ranibizumab, rituximab, tocilizumab, tositumomab, tralokinumab, tucotuzumab, trastuzumab, veltuzumab, zalutumumab, and zatuximab. Additional examples of recombinant antibodies that can be produced by the methods described herein are known in the art.

Examples of recombinant proteins that can be produced by the present methods include: alglucosidase alfa, laronidase, abatacept, galsulfase, lutropin alfa, antihemophilic factor, agalsidase beta, interferon beta-1a, darbepoetin alfa, tenecteplase, etanercept, coagulation factor IX, follicle stimulating hormone, interferon beta-1a, imiglucerase, dornase alfa, epoetin alfa, insulin or insulin analogs, mecasermin, factov VIII, factor VIIa, anti-thrombin III, protein C, human albumin, erythropoietin, granulocute colony stimulating factor, granulocyte macrophage colony stimulating factor, interleukin-11, laronidase, idursuphase, galsulphase, α-1-proteinase inhibitor, lactase, adenosine deaminase, tissue plasminogen activator, thyrotropin alpha, and alteplase. Additional examples include acid α-glucosidase, alglucosidase alpha, α-L-iduronidase, iduronate sulfatase, heparan N-sulfatase, galactose-6-sulfatase, acid β-galactosidase, β-glucoronidase, N-acetylglucosamine-1-phosphotransferase, α-N-acetylgalactosaminidase, acid lipase, lysosomal acid ceramidase, acid sphingomyelinase, β-glucosidase, galactosylceramidase, α-galactosidase-A, acid β-galactosidase, β-galactosidase, neuraminidase, hexosaminidase A, and hexosaminidase B. Further examples of recombinant proteins that can be produced by the methods described herein are known in the art.

In some embodiments of any of the methods described herein, the antibody is a human or a humanized antibody that binds to human complement protein C5. For example, the recombinant protein can be eculizumab consisting of a heavy chain comprising, consisting essentially of, or consisting of SEQ ID NO:1 and a light chain comprising, consisting essentially of, or consisting of SEQ ID NO:2. Nucleic acid that encodes the heavy and light chains of eculizumab are known in the art (see, for example, the nucleic acid sequences in U.S. Pat. No. 6,355,245 and Fc region sequences in An et al., mAbs 1:6, 572-579, 2009). In other examples, the recombinant protein can be Alexion 1210 consisting of a heavy chain comprising, consisting essentially of, or consisting of SEQ ID NO:3 and a light chain comprising, consisting essentially of, or consisting of SEQ ID NO:4. Nucleic acid that encodes the heavy and light chains of Alexion 1210 are known in the art (see, for example, the nucleic acid sequences in U.S. Patent Application Ser. No. 61/949,932).

### Cells and Cell Culture

Cells including a nucleic acid encoding a recombinant protein can be used to produce the recombinant protein (such as a secreted recombinant protein). In some examples, the nucleic acid encoding the recombinant protein is stably integrated into the genome of the cell. The cells used to produce the recombinant protein can be bacteria (e.g., gram negative bacteria), yeast (e.g., Saccharomyces cerevisiae, Pichia pastoris, Hansenula polymorpha, Kluyveromyces lactis, Schizosaccharomyces pombe, Yarrowia hpolytica, or Arxula adeninivorans), or mammalian cells.

The mammalian cell used to produce the recombinant protein can be a cell that grows in suspension or an adherent cell. Non-limiting examples of mammalian cells that can be cultured to produce a recombinant protein (e.g., any of the recombinant proteins described herein, such as eculizumab) include: Chinese hamster ovary (CHO) cells (such as CHO DG44 cells or CHO-K1s cells), Sp2.0, myeloma cells (such as NS/0 cells), B-cells, hybridoma cells, T-cells, human embryonic kidney (HEK) cells (such as HEK 293E and HEK 293F), African green monkey kidney epithelial cells (Vero) cells, and Madin-Darby Canine (Cocker Spaniel) kidney epithelial cells (MDCK) cells. In some examples where an adherent cell is used to produce a recombinant protein, the cell is cultured in the presence of a plurality of microcarriers (such as microcarriers that include one or more pores). Additional mammalian cells that can be cultured to produce a recombinant protein (such as a secreted recombinant protein) are known in the art. In some instances, the mammalian cell is cultured a bioreactor. In some embodiments, the mammalian cell used to inoculate a bioreactor was derived from a frozen cell stock or a seed train culture.

A nucleic acid encoding a recombinant protein can be introduced into a mammalian cell using a wide variety of methods known in molecular biology and molecular genetics. Non-limiting examples include transfection (e.g., lipofection), transduction (e.g., lentivirus, adenovirus, or retrovirus infection), and electroporation. In some instances, the nucleic acid is not stably integrated into a chromosome of the mammalian cell (transient transfection), while in others it is stably integrated into a chromosome of the mammalian cell. Alternatively or in addition, the nucleic acid can be present in a plasmid and/or in a mammalian artificial chromosome (such as a human artificial chromosome). Alternatively or in addition, the nucleic acid can be introduced into the cell using a viral vector (such as a lentivirus, retrovirus, or adenovirus vector). The nucleic acid can be operably linked to a promoter sequence (such as a strong promoter, such as a β-actin promoter and CMV promoter, or an inducible promoter). The nucleic acid can be operably linked to a heterologous promoter. A vector including the nucleic acid can, if desired, also include a selectable marker (such as a gene that confers hygromycin, puromycin, or neomycin resistance to the mammalian cell).

As noted herein, the recombinant protein can be a secreted protein that is released by the mammalian cell into the extracellular medium. For example, a nucleic acid sequence encoding a soluble recombinant protein can include a sequence that encodes a secretion signal peptide at the N- or C-terminus of the recombinant protein, which is cleaved by an enzyme present in the mammalian cell, and subsequently released into the extracellular medium (such as the first and/or second liquid culture medium in a perfusion cell culture or the first liquid culture medium and/or liquid feed culture medium in a feed batch culture).

Any of the methods described herein can further include culturing a mammalian cell including a nucleic acid encoding a recombinant protein under conditions sufficient to produce the recombinant protein (such as a secreted recombinant protein).

### Fed Batch Culturing

The culturing step in the methods described herein can include fed batch culturing. As is known in the art, fed batch culturing includes incremental (periodic) or continuous addition of a feed culture medium to an initial cell culture, which includes a first liquid culture medium, without substantial or significant removal of the first liquid culture medium from the cell culture. The cell culture in fed batch culturing can be disposed in a bioreactor (e.g., a production bioreactor, such as a 10,000-L production bioreactor). In some instances, the feed culture medium is the same as the first liquid culture medium. The feed culture medium may be either in a liquid form or a dry powder. In other instances, the feed culture medium is a concentrated form of the first liquid culture medium and/or is added as a dry powder. In some embodiments, both a first liquid feed culture medium and a different second liquid feed culture medium are added (e.g., continuously added) to the first liquid culture medium. In some examples, the addition of the first liquid feed culture medium and addition of the second liquid feed culture medium to the culture is initiated at about the same time. In some examples, the total volume of the first liquid feed culture medium and the second liquid feed culture medium added to the culture over the entire culturing period are about the same.

When the feed culture medium is added continuously, the rate of addition of the feed culture medium can be held constant or can be increased (e.g., steadily increased) over the culturing period. A continuous addition of feed culture medium can start at a specific time point during the culturing period (e.g., when the mammalian cells reach a target viable cell density, e.g., a viable cell density of about 1×10⁶ cells/mL, about 1.1×10⁶ cells/mL, about 1.2×10⁶ cells/mL, about 1.3×10⁶ cells/mL, about 1.4×10⁶ cells/mL, about 1.5×10⁶ cells/mL, about 1.6×10⁶ cells/mL, about 1.7×10⁶ cells/mL, about 1.8×10⁶ cells/mL, about 1.9×10⁶ cells/mL, or about 2.0×10⁶ cells/mL). In some embodiments, the continuous addition of feed culture medium can be initiated at day 2, day 3, day 4, or day 5 of the culturing period.

In some embodiments, an incremental (periodic) addition of feed culture medium can begin when the mammalian cells reach a target cell density (e.g., about 1×10⁶ cells/mL, about 1.1×10⁶ cells/mL, about 1.2×10⁶ cells/mL, about 1.3×10⁶ cells/mL, about 1.4×10⁶ cells/mL, about 1.5×10⁶ cells/mL, about 1.6×10⁶ cells/mL, about 1.7×10⁶ cells/mL, about 1.8×10⁶ cells/mL, about 1.9×10⁶, or about 2.0×10⁶ cells/mL). Incremental feed culture media addition can occur at regular intervals (e.g., every day, every other day, or every third day) or can occur when the cells reach specific target cell densities (e.g., target cell densities that increase over the culturing period). In some embodiments, the amount of feed culture medium added can progressively increase between the first incremental addition of feed culture medium and subsequent additions of feed culture medium. The volume of a liquid culture feed culture medium added to the initial cell culture over any 24 hour period in the culturing period can be some fraction of the initial volume of the bioreactor containing the culture or some fraction of the volume of the initial culture.

For example, the addition of the liquid feed culture medium (continuously or periodically) can occur at a time point that is between 6 hours and 7 days, between about 6 hours and about 6 days, between about 6 hours and about 5 days, between about 6 hours and about 4 days, between about 6 hours and about 3 days, between about 6 hours and about 2 days, between about 6 hours and about 1 day, between about 12 hours and about 7 days, between about 12 hours and about 6 days, between about 12 hours and about 5 days, between about 12 hours and about 4 days, between about 12 hours and about 3 days, between about 12 hours and about 2 days, between about 1 day and about 7 days, between about 1 day and about 6 days, between about 1 day and about 5 days, between about 1 day and about 4 days, between about 1 day and about 3 days, between about 1 day and about 2 days, between about 2 days and about 7 days, between about 2 days and about 6 days, between about 2 days and about 5 days, between about 2 days and about 4 days, between about 2 days and about 3 days, between about 3 days and about 7 days, between about 3 days and about 6 days, between about 3 days and about 5 days, between about 3 days and about 4 days, between about 4 days and about 7 days, between about 4 days and about 6 days, between about 4 days and about 5 days, between about 5 days and about 7 days, or between about 5 days and about 6 days, after the start of the culturing period.

The volume of a liquid feed culture medium added (continuously or periodically) to the initial cell culture over any 24 hour period can be between 0.01× and about 0.3× of the capacity of the bioreactor. In other embodiments, the volume of a liquid feed culture medium added (continuously or periodically) to the initial cell culture over any 24 hour period during the culturing period can be between 0.02× and about 1.0× of the volume of the initial cell culture. The total amount of feed culture medium added (continuously or periodically) over the entire culturing period can be between about 1% and about 40% of the volume of the initial culture.

In some examples, two different feed culture media are added (continuously or incrementally) during feed batch culturing. The amount or volume of the first feed culture medium and the second feed culture medium added can be substantially the same or can differ. The first feed culture medium can be in the form of a liquid and the second feed culture medium can be in the form of a solid, or vice-versa. The first feed culture medium and the second feed culture medium can be liquid feed culture media.

### Perfusion Culturing

The culturing step in the methods described herein can be perfusion culturing. As is known in the art, perfusion culturing includes removing from a bioreactor (e.g., a production bioreactor) a first volume of a first liquid culture medium, and adding to the production bioreactor a second volume of a second liquid culture medium, wherein the first volume and the second volume are typically (but need not be) about equal. The mammalian cells are retained in the bioreactor by some cell retention device or through techniques known in the art, such as cell settling. Removal and addition of culture media in perfusion culturing can be performed simultaneously or sequentially, or in some combination of the two. Further, removal and addition can be performed continuously, such as at a rate that removes and replaces a volume of between 0.1% to 800%, between 1% and 700%, between 1% and 600%, between 1% and 500%, between 1% and 400%, between 1% and 350%, between 1% and 300%, between 1% and 250%, between 1% and 100%, between 100% and 200%, between 5% and 150%, between 10% and 50%, between 15% and 40%, between 8% and 80%, or between 4% and 30% of the capacity of the bioreactor over an increment of time (such as over a 24-hour increment of time).

The first volume of the first liquid culture medium removed and the second volume of the second liquid culture medium added can in some instances be held approximately the same over each 24-hour period. As is known in the art, the rate at which the first volume of the first liquid culture medium is removed (volume/unit of time) and the rate at which the second volume of the second liquid culture medium is added (volume/unit of time) can be varied and depends on the conditions of the particular cell culture system. The rate at which the first volume of the first liquid culture medium is removed (volume/unit of time) and the rate at which the second volume of the second liquid culture medium is added (volume/unit of time) can be about the same or can be different.

Alternatively, the volume removed and added can change by gradually increasing over each 24-hour period. For example, the volume of the first liquid culture medium removed and the volume of the second liquid culture medium added within each 24-hour period can be increased over the culturing period. The volume can be increased a volume that is between 0.5% to about 20% of the capacity of the bioreactor over a 24-hour period. The volume can be increased over the culturing period to a volume that is about 25% to about 150% of the capacity of the bioreactor or the first liquid culture medium volume over a 24-hour period.

In some examples of the methods described herein, after the first 48 to 96 hours of the culturing period, in each 24-hour period, the first volume of the first liquid culture medium removed and the second volume of the second liquid culture medium added is about 10% to about 95%, about 10% to about 20%, about 20% to about 30%, about 30% to about 40%, about 40% to about 50%, about 50% to about 60%, about 60% to about 70%, about 70% to about 80%, about 80% to about 90%, about 85% to about 95%, about 60% to about 80%, or about 70% of the volume of the first liquid culture medium.

Skilled practitioners will appreciate that the first liquid culture medium and the second liquid culture medium can be the same type of media. In other instances, the first liquid culture medium and the second liquid culture medium can be different. The second liquid culture medium may be more concentrated with respect to one or more media components. In some embodiments, the first liquid culture medium includes processed BSA, the second liquid culture medium includes processed BSA, or both the first and the second liquid culture medium include processed BSA.

The first volume of the first liquid culture medium can be removed using any method, e.g., using an automated system. For example, alternating tangential flow filtration may be used. Alternatively, the first volume of the first liquid culture medium can be removed by seeping or gravity flow of the first volume of the first liquid culture medium through a sterile membrane with a molecular weight cut-off that excludes the mammalian cell. Alternatively, the first volume of the first liquid culture medium can be removed by stopping or significantly decreasing the rate of agitation for a period of at least 1 minute, at least 2 minutes, 3 minutes, 4 minutes, 5 minutes, 10 minutes, 15 minutes, 20 minutes, 25 minutes, 30 minutes, 40 minutes, 50 minutes, or 1 hour, and removing or aspirating the first volume of the first liquid culture medium from the top of the production bioreactor.

The second volume of the second liquid culture medium can be added to the first liquid culture medium by a pump. The second liquid culture medium can be added to the first liquid medium manually, such as by pipetting or injecting the second volume of the second liquid culture medium directly onto the first liquid culture medium or in an automated fashion.

### Liquid Culture Medium and Clarification

A solution comprising a recombinant protein, e.g., a liquid culture medium comprising a recombinant protein and that is substantially free of cells, can be derived from any source. For example, the liquid culture medium can be obtained from a recombinant cell culture (such as a recombinant bacterial, yeast, or mammalian cell culture). The liquid culture medium can be obtained from a fed-batch mammalian cell culture (such as a fed-batch bioreactor containing a culture of mammalian cells that secrete the recombinant protein) or a perfusion cell mammalian cell culture (such as a perfusion bioreactor containing a culture of mammalian cells that secrete the recombinant protein). The liquid culture medium can be a clarified liquid culture medium from a culture of bacterial, yeast, or mammalian cells that secrete the recombinant protein.

Liquid culture medium obtained from a recombinant cell culture can be clarified to obtain a liquid culture medium that is substantially free of cells and that includes a recombinant protein (also called a clarified culture medium or clarified liquid culture medium). Methods for clarifying a liquid culture medium in order to remove cells are known in the art (such as through the use of 0.2-μm filtration and filtration using an Alternating Tangential Flow (ATF′) system or tangential flow filtration (TFF)). Recombinant cells can be removed from liquid culture medium using centrifugation and removing the supernatant or by allowing the cells to settle to the gravitational bottom of a container (such as a bioreactor) and removing the liquid culture medium that is substantially free of cells. The liquid culture medium can be obtained from a culture of recombinant cells (such as recombinant bacteria, yeast, or mammalian cells) producing any of the recombinant proteins described herein.

The liquid culture medium including a recombinant protein or the liquid culture media used to culture a mammalian cell including a nucleic acid encoding a recombinant protein (such as the first and second liquid culture medium in perfusion culturing or the first liquid culture medium and the liquid feed culture medium in fed batch culturing) can be any of the types of liquid culture medium described herein or known in the art. For example, any of the liquid culture media described herein can be selected from the group of: animal-derived component free liquid culture medium, serum-free liquid culture medium, serum-containing liquid culture medium, chemically-defined liquid culture medium, and protein-free liquid culture medium. In any of the processes described herein, a liquid culture medium obtained from a culture can be diluted by addition of a second fluid (such as a buffered solution) before or after it is clarified and/or before the recombinant protein is captured.

The liquid culture medium that includes a recombinant protein and is substantially free of cells can be stored (such as at a temperature below about 15° C., below about 10° C., below about 4° C., below about 0° C., below about −20° C., below about −50° C., below about −70 C.°, or below about −80° C.) for at least or about 1 day, at least or about 2 days, at least or about 5 days, at least or about 10 days, at least or about 15 days, at least or about 20 days, or at least or about 30 days) prior to capturing the recombinant protein from the liquid culture medium. Alternatively, in some examples, the recombinant protein is captured from the liquid culture medium directly from a bioreactor after a clarification step.

### Capturing the Recombinant Protein

The methods provided herein include a step of capturing a recombinant protein from a solution including the recombinant protein (such as a clarified liquid culture medium comprising the secreted recombinant protein or a clarified liquid culture medium comprising the recombinant protein that has been diluted with a buffered solution).

As can be appreciated in the art, through performance of the capturing step, the recombinant protein can be partially purified or isolated (e.g., at least or about 5%, e.g., at least or about 10%, 15%, 20%, 25%, 30%, 40%, 45%, 50%, 55%, 60%, 65%, 70%, 75%, 80%, 85%, 90%, or at least or about 95% pure by weight), concentrated, and stabilized from one or more other components present in a clarified liquid culture medium comprising the recombinant protein (such as culture medium proteins or one or more other components (such as DNA, RNA, or other proteins) present in or secreted from a mammalian cell). Typically, capturing is performed using a resin that binds a recombinant protein (such as through the use of affinity chromatography). Non-limiting examples of methods for capturing a recombinant protein from a solution including the recombinant protein (such as a clarified liquid culture medium) are described herein and others are known in the art. In the methods described herein, a recombinant protein can be captured from a solution using at least one chromatography column (such as any of the chromatography columns and/or capture mechanisms described herein, such as affinity chromatography resin, an anionic exchange chromatography resin, a cationic exchange chromatography resin, a mixed-mode chromatography resin, a molecular sieve chromatography resin, or a hydrophobic interaction chromatography resin). The capturing step can be performed using a chromatography resin that utilizes a protein A-binding capture mechanism, an antibody- or antibody fragment-binding capture mechanism, a substrate binding capture mechanism, and a cofactor-binding capture mechanism.

For example, if the recombinant protein is an antibody or an antibody fragment, the capturing system can be a protein A-binding capturing mechanism or an antigen-binding capturing mechanism (where the capturing antigen is specifically recognized by the recombinant therapeutic antibody or antibody fragment). If the recombinant protein is an enzyme, the capturing mechanism can use an antibody or antibody fragment that specifically binds to the enzyme to capture the recombinant enzyme, a substrate of the enzyme to capture the recombinant enzyme, a cofactor of the enzyme to capture the recombinant enzyme, or, if the recombinant enzyme includes a tag, a protein, metal chelate, or antibody (or antibody fragment) that specifically binds to the tag present in the recombinant enzyme. Non-limiting examples of resins that can be used to capture a recombinant protein are described herein and additional resins are known in the art. Non-limiting examples of resin that utilize a protein A-binding capture mechanism is MABSELECT™ SURE™ resin and Protein A Sepharose™ CL-4B (GE Healthcare).

Exemplary non-limiting sizes and shapes of the chromatography column(s) that can be used to capture the recombinant protein are well known in the art. The liquid culture medium fed (loaded) can include, for example, between about 0.05 mg/mL to about 100 mg/mL recombinant protein, between about 0.1 mg/mL to about 90 mg/mL, between about 0.1 mg/mL to about 80 mg/mL, between about 0.1 mg/mL to about 70 mg/mL, between about 0.1 mg/mL to about 60 mg/mL, between about 0.1 mg/mL to about 50 mg/mL, between about 0.1 mg/mL to about 40 mg/mL, between about 0.1 mg/mL to about 30 mg/mL, between about 0.1 mg/mL to about 20 mg/mL, between 0.5 mg/mL to about 20 mg/mL, between about 0.1 mg/mL to about 15 mg/mL, between about 0.5 mg/mL to about 15 mg/mL, between about 0.1 mg/mL to about 10 mg/mL, or between about 0.5 mg/mL to about 10 mg/mL recombinant protein.

As can be appreciated in the art, to capture the recombinant protein using the chromatography column(s), one must perform the sequential chromatographic steps of loading, washing, eluting, and regenerating the chromatography column(s) or chromatography membrane(s).

Following the loading of the recombinant protein onto the at least one chromatographic column that includes a resin that is capable of capturing the recombinant protein, the at least one chromatographic column or chromatographic membrane is washed with at least one washing buffer. As can be appreciated in the art, the at least one (such as two, three, or four) washing buffer is meant to elute all proteins that are not the recombinant protein from the at least one chromatography column, while not disturbing the interaction of the recombinant protein with the resin.

Following washing, the recombinant protein is eluted from the at least one chromatographic column or chromatographic membrane by passing an elution buffer through the at least one chromatographic column or chromatographic membrane. Non-limiting examples of elution buffers that can be used in these methods will depend on the capture mechanism and/or the recombinant protein. For example, an elution buffer can include a different concentration of salt (e.g., increased salt concentration), a different pH (e.g., an increased or decreased salt concentration), or a molecule that will compete with the recombinant protein for binding to the resin that is capable of performing the unit operation of capturing. Examples of such elution buffers for each exemplary capture mechanism described herein are well known in the art.

Following elution of the recombinant protein from the at least one chromatographic column that includes a resin that is capable of capturing the recombinant protein, and before the next volume of solution including the recombinant protein can be loaded onto the at least one chromatographic column, the at least one chromatography column or chromatographic membrane must be equilibrated using an regeneration buffer.

### Depth Filtration

The methods include a step of flowing the recombinant protein through a depth filter to provide a filtrate that comprises the purified recombinant protein and is substantially free of soluble protein aggregates. Any of the exemplary depth filters or methods for depth filtration described herein can be used to flow a recombinant protein through a depth filter.

In some embodiments of the methods described herein, the recombinant protein is flowed through the depth filter in a solution having a pH of between about 4.0 to about 8.5, between about 4.0 to about 8.4, between about 4.0 to about 8.2, between about 4.0 to about 8.0, between about 4.0 to about 8.0, between about 4.0 to about 7.8, between about 4.0 to about 7.6, between about 4.0 to about 7.5, between about 4.0 to about 7.4, between about 4.0 to about 7.2, between about 4.0 to about 7.0, between about 4.0 to about 6.8, between about 4.0 to about 6.6, between about 4.0 to about 6.4, between about 4.0 to about 6.2, between about 4.0 to about 6.0, between about 4.0 to about 5.8, between about 4.0 to about 5.6, between about 4.0 to about 5.4, between about 4.0 to about 5.2, between about 4.0 to about 5.0, between about 4.0 to about 4.8, between about 4.0 to about 4.6, between about 4.0 to about 4.4, between about 4.0 to about 4.2, between about 4.2 to about 8.5, between about 4.2 to about 8.4, between about 4.2 to about 8.2, between about 4.2 to about 8.0, between about 4.2 to about 7.8, between about 4.2 to about 7.6, between about 4.2 to about 7.5, between about 4.2 to about 7.4, between about 4.2 to about 7.2, between about 4.2 to about 7.0, between about 4.2 to about 6.8, between about 4.2 to about 6.6, between about 4.2 to about 6.4, between about 4.2 to about 6.2, between about 4.2 to about 6.0, between about 4.2 to about 5.8, between about 4.2 to about 5.6, between about 4.2 to about 5.4, between about 4.2 to about 5.2, between about 4.2 to about 5.0, between about 4.2 to about 4.8, between about 4.2 to about 4.6, between about 4.2 to about 4.4, between about 4.4 to about 8.5, between about 4.4 to about 8.4, between about 4.4 to about 8.2, between about 4.4 to about 8.0, between about 4.4 to about 7.8, between about 4.4 to about 7.6, between about 4.4 to about 7.5, between about 4.4 to about 7.4, between about 4.4 to about 7.2, between about 4.4 to about 7.0, between about 4.4 to about 6.8, between about 4.4 to about 6.6, between about 4.4 to about 6.4, between about 4.4 to about 6.2, between about 4.4 to about 6.0, between about 4.4 to about 5.8, between about 4.4 to about 5.6, between about 4.4 to about 5.4, between about 4.4 to about 5.2, between about 4.4 to about 5.0, between about 4.4 to about 4.8, between about 4.4 to about 4.6, between about 4.6 to about 8.5, between about 4.6 to about 8.4, between about 4.6 to about 8.2, between about 4.6 to about 8.0, between about 4.6 to about 7.8, between about 4.6 to about 7.6, between about 4.6 to about 7.5, between about 4.6 to about 7.4, between about 4.6 to about 7.2, between about 4.6 to about 7.0, between about 4.6 to about 6.8, between about 4.6 to about 6.6, between about 4.6 to about 6.4, between about 4.6 to about 6.2, between about 4.6 to about 6.0, between about 4.6 to about 5.8, between about 4.6 to about 5.6, between about 4.6 to about 5.4, between about 4.6 to about 5.2, between about 4.6 to about 5.0, between about 4.6 to about 4.8, between about 4.8 to about 8.5, between about 4.8 to about 8.4, between about 4.8 to about 8.2, between about 4.8 to about 8.0, between about 4.8 to about 7.8, between about 4.8 to about 7.6, between about 4.8 to about 7.5, between about 4.8 to about 7.4, between about 4.8 to about 7.2, between about 4.8 to about 7.0, between about 4.8 to about 6.8, between about 4.8 to about 6.6, between about 4.8 to about 6.4, between about 4.8 to about 6.2, between about 4.8 to about 6.0, between about 4.8 to about 5.8, between about 4.8 to about 5.6, between about 4.8 to about 5.4, between about 4.8 to about 5.2, between about 4.8 to about 5.0, between about 5.0 to about 8.5, between about 5.0 to about 8.4, between about 5.0 to about 8.2, between about 5.0 to about 8.0, between about 5.0 to about 7.8, between about 5.0 to about 7.6, between about 5.0 to about 7.5, between about 5.0 to about 7.2, between about 5.0 to about 7.0, between about 5.0 to about 6.8, between about 5.0 to about 6.6, between about 5.0 to about 6.4, between about 5.0 to about 6.2, between about 5.0 to about 6.0, between about 5.0 to about 5.8, between about 5.0 to about 5.6, between about 5.0 to about 5.4, between about 5.0 to about 5.2, between about 5.2 to about 8.5, between about 5.2 to about 8.4, between about 5.2 to about 8.2, between about 5.2 to about 8.0, between about 5.2 to about 7.8, between about 5.2 to about 7.6, between about 5.2 to about 7.5, between about 5.2 to about 7.4, between about 5.2 to about 7.2, between about 5.2 to about 7.0, between about 5.2 to about 6.8, between about 5.2 to about 6.6, between about 5.2 to about 6.4, between about 5.2 to about 6.2, between about 5.2 to about 6.0, between about 5.2 to about 5.8, between about 5.2 to about 5.6, between about 5.2 to about 5.4, between about 5.4 to about 8.5, between about 5.4 to about 8.4, between about 5.4 to about 8.2, between about 5.4 to about 8.0, between about 5.4 to about 7.8, between about 5.4 to about 7.6, between about 5.4 to about 7.5, between about 5.4 to about 7.4, between about 5.4 to about 7.2, between about 5.4 to about 7.0, between about 5.4 to about 6.8, between about 5.4 to about 6.6, between about 5.4 to about 6.4, between about 5.4 to about 6.2, between about 5.4 to about 6.0, between about 5.4 to about 5.8, between about 5.4 to about 5.6, between about 5.6 to about 8.5, between about 5.6 to about 8.4, between about 5.6 to about 8.2, between about 5.6 to about 8.0, between about 5.6 to about 7.8, between about 5.6 to about 7.6, between about 5.6 to about 7.5, between about 5.6 to about 7.4, between about 5.6 to about 7.2, between about 5.6 to about 7.0, between about 5.6 to about 6.8, between about 5.6 to about 6.6, between about 5.6 to about 6.4, between about 5.6 to about 6.2, between about 5.6 to about 6.0, between about 5.6 to about 5.8, between about 5.8 to about 8.5, between about 5.8 to about 8.4, between about 5.8 to about 8.2, between about 5.8 to about 8.0, between about 5.8 to about 7.8, between about 5.8 to about 7.6, between about 5.8 to about 7.5, between about 5.8 to about 7.4, between about 5.8 to about 7.2, between about 5.8 to about 7.0, between about 5.8 to about 6.8, between about 5.8 to about 6.6, between about 5.8 to about 6.4, between about 5.8 to about 6.2, between about 5.8 to about 6.0, between about 6.0 to about 8.5, between about 6.0 to about 8.4, between about 6.0 to about 8.2, between about 6.0 to about 8.0, between about 6.0 to about 7.8, between about 6.0 to about 7.6, between about 6.0 to about 7.5, between about 6.0 to about 7.4, between about 6.0 to about 7.2, between about 6.0 to about 7.0, between about 6.0 to about 6.8, between about 6.0 to about 6.6, between about 6.0 to about 6.4, between about 6.0 to about 6.2, between about 6.2 to about 8.5, between 6.2 to about 8.4, between 6.2 to about 8.2, between 6.2 to about 8.0, between 6.2 to about 7.8, between 6.2 to about 7.6, between about 6.2 to about 7.5, between about 6.2 to about 7.4, between about 6.2 to about 7.2, between about 6.2 to about 7.0, between about 6.2 to about 6.8, between about 6.2 to about 6.6, between about 6.2 to about 6.4, between 6.4 to about 8.5, between 6.4 to about 8.4, between 6.4 to about 8.2, between 6.4 to about 8.0, between 6.4 to about 7.8, between 6.4 to about 7.6, between about 6.4 to about 7.5, between about 6.4 to about 7.4, between about 6.4 to about 7.2, between about 6.4 to about 7.0, between about 6.4 to about 6.8, between about 6.4 to about 6.6, between 6.6 to about 8.5, between 6.6 to about 8.4, between 6.6 to about 8.2, between 6.6 to about 8.0, between 6.6 to about 7.8, between 6.6 to about 7.6, between about 6.6 to about 7.5, between about 6.6 to about 7.4, between about 6.6 to about 7.2, between about 6.6 to about 7.0, between about 6.6 to about 6.8, between 6.8 to about 8.5, between 6.8 to about 8.4, between 6.8 to about 8.2, between 6.8 to about 8.0, between 6.8 to about 7.8, between 6.8 to about 7.6, between about 6.8 to about 7.5, between about 6.8 to about 7.4, between about 6.8 to about 7.2, between about 6.8 to about 7.0, between about 7.0 to about 8.5, between about 7.0 to about 8.4, between about 7.0 to about 8.2, between about 7.0 to about 8.0, between about 7.0 to about 7.8, between about 7.0 to about 7.6, between about 7.0 to about 7.5, between about 7.0 to about 7.4, between about 7.0 to about 7.2, between about 7.2 to about 8.5, between about 7.2 to about 8.4, between about 7.2 to about 8.2, between about 7.2 to about 8.0, between about 7.2 to about 7.8, between about 7.2 to about 7.6, between about 7.2 to about 7.5, between about 7.2 to about 7.4, between 7.4 to about 8.5, between about 7.4 to about 8.4, between about 7.4 to about 8.2, between about 7.4 to about 8.0, between about 7.4 to about 7.8, between about 7.4 to about 7.6, between about 7.6 to about 8.5, between about 7.6 to about 8.4, between about 7.6 to about 8.2, between about 7.6 to about 8.0, between about 7.6 to about 7.8, between about 7.8 to about 8.5, between about 7.8 to about 8.4, between about 7.8 to about 8.2, between about 7.8 to about 8.0, between about 8.0 to about 8.5, between about 8.0 to about 8.3, between about 8.0 to about 8.2, between about 8.2 to about 8.5, between about 8.2 to about 8.4, or between about 8.3 to about 8.5.

The solution including the recombinant protein that is flowed through a depth filter can include a concentration of recombinant protein between about 0.01 mg/mL and about 25 mg/mL (e.g., between about 0.01 mg/mL and about 22.5 mg/mL, between about 0.01 mg/mL and about 20.0 mg/mL, between about 0.01 mg/mL and about 17.5 mg/mL, between about 0.01 mg/mL and about 15.0 mg/mL, between about 0.01 mg/mL and about 12.5 mg/mL, between about 0.01 mg/mL and about 10 mg/mL, between about 0.01 mg/mL and about 8 mg/mL, between about 0.01 mg/mL and about 6 mg/mL, between about 0.01 mg/mL and about 5 mg/mL, between about 0.01 mg/mL and about 4 mg/mL, between about 0.01 mg/mL and about 3.5 mg/mL, between about 0.01 mg/mL and about 3.0 mg/mL, between about 0.01 mg/mL and about 2.5 mg/mL, between about 0.01 mg/mL and about 2.0 mg/mL, between about 0.01 mg/mL and about 1.5 mg/mL, between about 0.01 mg/mL and about 1.0 mg/mL, between about 0.01 mg/mL and about 0.5 mg/mL, between about 0.01 mg/mL and about 0.25 mg/mL, between about 0.01 mg/mL and about 0.1 mg/mL, between about 0.1 mg/mL and about 12.5 mg/mL, between about 0.1 mg/mL and about 10.0 mg/mL, between about 0.1 mg/mL and about 8.0 mg/mL, between about 0.1 mg/mL and about 6.0 mg/mL, between about 0.1 mg/mL and about 5.0 mg/mL, between about 0.1 mg/mL and about 4.0 mg/mL, between about 0.1 mg/mL and about 3.5 mg/mL, between about 0.1 mg/mL and about 3.0 mg/mL, between about 0.1 mg/mL and about 2.5 mg/mL, between about 0.1 mg/mL and about 2.0 mg/mL, between about 0.1 mg/mL and about 1.5 mg/mL, between about 0.1 mg/mL and about 1.0 mg/mL, between about 0.1 mg/mL and about 0.5 mg/mL, or between about 0.1 mg/mL and about 0.25 mg/mL).

In some embodiments, a solution comprising the recombinant protein is flowed through the depth filter at a flow rate of between about 25 L/m²/h to about 400 L/m²/h, between about 25 L/m²/h to about 390 L/m²/h, between about 25 L/m²/h to about 380 L/m²/h, between about 25 L/m²/h to about 360 L/m²/h, between about 25 L/m²/h to about 340 L/m²/h, between about 25 L/m²/h to about 320 L/m²/h, between about 25 L/m²/h to about 300 L/m²/h, between about 25 L/m²/h to about 280 L/m²/h, between about 25 L/m²/h to about 260 L/m²/h, between about 25 L/m²/h to about 240 L/m²/h, between about 25 L/m²/h to about 220 L/m²/h, between about 25 L/m²/h to about 200 L/m²/h, between about 25 L/m²/h to about 180 L/m²/h, between about 25 L/m²/h to about 160 L/m²/h, between about 25 L/m²/h to about 140 L/m²/h, between about 25 L/m²/h to about 120 L/m²/h, between about 25 L/m²/h to about 100 L/m²/h, between about 25 L/m²/h to about 80 L/m²/h, between about 25 L/m²/h to about 60 L/m²/h, between about 25 L/m²/h to about 40 L/m²/h, between about 25 L/m²/h to about 35 L/m²/h, between about 40 L/m²/h to about 400 L/m²/h, between about 40 L/m²/h to about 380 L/m²/h, between about 40 L/m²/h to about 360 L/m²/h, between about 40 L/m²/h to about 340 L/m²/h, between about 40 L/m²/h to about 320 L/m²/h, between about 40 L/m²/h to about 300 L/m²/h, between about 40 L/m²/h to about 280 L/m²/h, between about 40 L/m²/h to about 260 L/m²/h, between about 40 L/m²/h to about 240 L/m²/h, between about 40 L/m²/h to about 220 L/m²/h, between about 40 L/m²/h to about 220 L/m²/h, between about 40 L/m²/h to about 200 L/m²/h, between about 40 L/m²/h to about 180 L/m²/h, between about 40 L/m²/h to about 160 L/m²/h, between about 40 L/m²/h to about 140 L/m²/h, between about 40 L/m²/h to about 120 L/m²/h, between about 40 L/m²/h to about 100 L/m²/h, between about 40 L/m²/h to about 80 L/m²/h, between about 40 L/m²/h to about 60 L/m²/h, between about 40 L/m²/h to about 50 L/m²/h, between about 60 L/m²/h to about 400 L/m²/h, between about 60 L/m²/h to about 380 L/m²/h, between about 60 L/m²/h to about 360 L/m²/h, between about 60 L/m²/h to about 340 L/m²/h, between about 60 L/m²/h to about 320 L/m²/h, between about 60 L/m²/h to about 300 L/m²/h, between about 60 L/m²/h to about 280 L/m²/h, between about 60 L/m²/h to about 260 L/m²/h, between about 60 L/m²/h to about 240 L/m²/h, between about 60 L/m²/h to about 220 L/m²/h, between about 60 L/m²/h to about 200 L/m²/h, between about 60 L/m²/h to about 180 L/m²/h, between about 70 L/m²/h to about 150 L/m²/h, between about 70 L/m²/h to about 180 L/m²/h, between about 60 L/m²/h to about 160 L/m²/h, between about 60 L/m²/h to about 140 L/m²/h, between about 60 L/m²/h to about 120 L/m²/h, between about 60 L/m²/h to about 100 L/m²/h, between about 60 L/m²/h to about 80 L/m²/h, between about 80 L/m²/h to about 400 L/m²/h, between about 80 L/m²/h to about 380 L/m²/h, between about 80 L/m²/h to about 360 L/m²/h, between about 80 L/m²/h to about 340 L/m²/h, between about 80 L/m²/h to about 320 L/m²/h, between about 80 L/m²/h to about 300 L/m²/h, between about 80 L/m²/h to about 280 L/m²/h, between about 80 L/m²/h to about 260 L/m²/h, between about 80 L/m²/h to about 240 L/m²/h, between about 80 L/m²/h to about 220 L/m²/h. between about 80 L/m²/h to about 200 L/m²/h, between about 80 L/m²/h to about 180 L/m²/h, between about 80 L/m²/h to about 160 L/m²/h, between about 80 L/m²/h to about 140 L/m²/h, between about 80 L/m²/h to about 120 L/m²/h, between about 80 L/m²/h to about 100 L/m²/h, between about 100 L/m²/h to about 400 L/m²/h, between about 100 L/m²/h to about 380 L/m²/h, between about 100 L/m²/h to about 360 L/m²/h, between about 100 L/m²/h to about 340 L/m²/h, between about 100 L/m²/h to about 320 L/m²/h, between about 100 L/m²/h to about 300 L/m²/h, between about 100 L/m²/h to about 280 L/m²/h, between about 100 L/m²/h to about 260 L/m²/h, between about 100 L/m²/h to about 240 L/m²/h, between about 100 L/m²/h to about 220 L/m²/h, between about 100 L/m²/h to about 200 L/m²/h, 100 L/m²/h to about 180 L/m²/h, between about 100 L/m²/h to about 160 L/m²/h, between about 100 L/m²/h to about 140 L/m²/h, between about 100 L/m²/h to about 120 L/m²/h, between about 120 L/m²/h to about 400 L/m²/h, between about 120 L/m²/h to about 380 L/m²/h, between about 120 L/m²/h to about 360 L/m²/h, between about 120 L/m²/h to about 340 L/m²/h, between about 120 L/m²/h to about 320 L/m²/h, between about 120 L/m²/h to about 300 L/m²/h, between about 120 L/m²/h to about 280 L/m²/h, between about 120 L/m²/h to about 260 L/m²/h, between about 120 L/m²/h to about 240 L/m²/h, between about 120 L/m²/h to about 220 L/m²/h, between about 120 L/m²/h to about 200 L/m²/h, between about 120 L/m²/h to about 180 L/m²/h, between about 120 L/m²/h to about 160 L/m²/h, between about 120 L/m²/h to about 140 L/m²/h, between about 140 L/m²/h to about 400 L/m²/h, between about 140 L/m²/h to about 380 L/m²/h, between about 140 L/m²/h to about 360 L/m²/h, between about 140 L/m²/h to about 340 L/m²/h, between about 140 L/m²/h to about 320 L/m²/h, between about 140 L/m²/h to about 300 L/m²/h, between about 140 L/m²/h to about 280 L/m²/h, between about 140 L/m²/h to about 260 L/m²/h, between about 140 L/m²/h to about 240 L/m²/h, between about 140 L/m²/h to about 220 L/m²/h, between about 140 L/m²/h to about 200 L/m²/h, between about 140 L/m²/h to about 180 L/m²/h, between about 140 L/m²/h to about 160 L/m²/h, between about 160 L/m²/h to about 400 L/m²/h, between about 160 L/m²/h to about 380 L/m²/h, between about 160 L/m²/h to about 360 L/m²/h, between about 160 L/m²/h to about 340 L/m²/h, between about 160 L/m²/h to about 320 L/m²/h, between about 160 L/m²/h to about 300 L/m²/h, between about 160 L/m²/h to about 280 L/m²/h, between about 160 L/m²/h to about 260 L/m²/h, between about 160 L/m²/h to about 240 L/m²/h, between about 160 L/m²/h to about 220 L/m²/h, between about 160 L/m²/h to about 200 L/m²/h, between about 160 L/m²/h to about 180 L/m²/h, between about 180 L/m²/h to about 400 L/m²/h, between about 180 L/m²/h to about 380 L/m²/h, between about 180 L/m²/h to about 360 L/m²/h, between about 180 L/m²/h to about 340 L/m²/h, between about 180 L/m²/h to about 320 L/m²/h, between about 180 L/m²/h to about 300 L/m²/h, between about 180 L/m²/h to about 280 L/m²/h, between about 180 L/m²/h to about 260 L/m²/h, between about 180 L/m²/h to about 240 L/m²/h, between about 180 L/m²/h to about 220 L/m²/h, between about 180 L/m²/h to about 200 L/m²/h, between about 200 L/m²/h to about 400 L/m²/h, between about 200 L/m²/h to about 380 L/m²/h, between about 200 L/m²/h to about 360 L/m²/h, between about 200 L/m²/h to about 340 L/m²/h, between about 200 L/m²/h to about 320 L/m²/h, between about 200 L/m²/h to about 300 L/m²/h, between about 200 L/m²/h to about 280 L/m²/h, between about 200 L/m²/h to about 260 L/m²/h, between about 200 L/m²/h to about 240 L/m²/h, between about 200 L/m²/h to about 220 L/m²/h, between about 220 L/m²/h to about 400 L/m²/h, between about 220 L/m²/h to about 380 L/m²/h, between about 220 L/m²/h to about 360 L/m²/h, between about 220 L/m²/h to about 340 L/m²/h, between about 220 L/m²/h to about 320 L/m²/h, between about 220 L/m²/h to about 300 L/m²/h, between about 220 L/m²/h to about 280 L/m²/h, between about 220 L/m²/h to about 260 L/m²/h, between about 220 L/m²/h to about 240 L/m²/h, between about 240 L/m²/h to about 400 L/m²/h, between about 240 L/m²/h to about 380 L/m²/h, between about 240 L/m²/h to about 360 L/m²/h, between about 240 L/m²/h to about 340 L/m²/h, between about 240 L/m²/h to about 320 L/m²/h, between about 240 L/m²/h to about 300 L/m²/h, between about 240 L/m²/h to about 280 L/m²/h, between about 240 L/m²/h to about 260 L/m²/h, between about 260 L/m²/h to about 400 L/m²/h, between about 260 L/m²/h to about 380 L/m²/h, between about 260 L/m²/h to about 360 L/m²/h, between about 260 L/m²/h to about 340 L/m²/h, between about 260 L/m²/h to about 320 L/m²/h, between about 260 L/m²/h to about 300 L/m²/h, between about 260 L/m²/h to about 280 L/m²/h, between about 280 L/m²/h to about 400 L/m²/h, between about 280 L/m²/h to about 380 L/m²/h, between about 280 L/m²/h to about 360 L/m²/h, between about 280 L/m²/h to about 340 L/m²/h, between about 280 L/m²/h to about 320 L/m²/h, between about 280 L/m²/h to about 300 L/m²/h, between about 300 L/m²/h to about 400 L/m²/h, between about 300 L/m²/h to about 380 L/m²/h, between about 300 L/m²/h to about 360 L/m²/h. between about 300 L/m²/h to about 340 L/m²/h, between about 300 L/m²/h to about 320 L/m²/h, between about 320 L/m²/h to about 400 L/m²/h, between about 320 L/m²/h to about 380 L/m²/h, between about 320 L/m²/h to about 360 L/m²/h, between about 320 L/m²/h to about 340 L/m²/h, between about 340 L/m²/h to about 400 L/m²/h, between about 340 L/m²/h to about 380 L/m²/h, between about 340 L/m²/h to about 360 L/m²/h, between about 360 L/m²/h to about 400 L/m²/h, between about 360 L/m²/h to about 380 L/m²/h, or between about 380 L/m²/h to about 400 L/m²/h, to selectively retain soluble protein aggregates, such as protein dimers and higher protein oligomers (such as soluble recombinant protein aggregates). This filtration step is performed using a depth filter including a filtration media of, for example, silica, one or more layers of a fibrous media, one or more layers of charged or surface modified microporous membranes, or a small bed of chromatography media. The depth filters described herein can have a membrane surface area of between about 10 cm² to about 32000 cm², between about 10 cm² to about 31000 cm², between about 10 cm² to about 30000 cm², between about 10 cm² to about 29000 cm², between about 10 cm² to about 28000 cm², between about 10 cm² to about 27000 cm², between about 10 cm² to about 26000 cm², between about 10 cm² to about 25000 cm², between about 10 cm² to about 24000 cm², between about 10 cm² to about 23000 cm², between about 10 cm² to about 22000 cm², between about 10 cm² to about 21000 cm², between about 10 cm² to about 20000 cm², between about 10 cm² to about 19000 cm², between about 10 cm² to about 18000 cm², between about 10 cm² to about 17000 cm², between about 10 cm² to about 16000 cm², between about 10 cm² to about 15000 cm², between about 10 cm² to about 14000 cm², between about 10 cm² to about 13000 cm², between about 10 cm² to about 12000 cm², between about 10 cm² to about 11000 cm², between about 10 cm² to about 10000 cm², between about 10 cm² to about 9000 cm², between about 10 cm² to about 8000 cm², between about 10 cm² to about 7000 cm², between about 10 cm² to about 6000 cm², between about 10 cm² to about 5000 cm², between about 10 cm² to about 4000 cm², between about 10 cm² to about 3000 cm², between about 10 cm² to about 2000 cm², between about 10 cm² to about 1500 cm², between about 10 cm² to about 1020 cm², between about 10 cm² to about 1000 cm², between about 10 cm² to about 500 cm², between about 10 cm² to about 75 cm², between about 100 cm² to about 25000 cm², between about 100 cm² to about 24000 cm², between about 100 cm² to about 23000 cm², between about 100 cm² to about 22000 cm², between about 100 cm² to about 21000 cm², between about 100 cm² to about 20000 cm², between about 100 cm² to about 19000 cm², between about 100 cm² to about 18000 cm², between about 100 cm² to about 17000 cm², between about 100 cm² to about 16000 cm², between about 100 cm² to about 15000 cm², between about 100 cm² to about 14000 cm², between about 100 cm² to about 13000 cm², between about 100 cm² to about 12000 cm², between about 100 cm² to about 11000 cm², between about 100 cm² to about 10000 cm², between about 1000 cm² to about 9000 cm², between about 100 cm² to about 8000 cm², between about 100 cm² to about 7000 cm², between about 100 cm² to about 6000 cm², between about 100 cm² to about 5000 cm², between about 100 cm² to about 4000 cm², between about 100 cm² to about 3000 cm², between about 100 cm² to about 2000 cm², between about 100 cm² to about 1000 cm², between about 100 cm² to about 500 cm², between about 500 cm² to about 25000 cm², between about 500 cm² to about 24000 cm², between about 500 cm² to about 23000 cm², between about 500 cm² to about 22000 cm², between about 500 cm² to about 21000 cm², between about 500 cm² to about 20000 cm², between about 500 cm² to about 19000 cm², between about 500 cm² to about 18000 cm², between about 500 cm² to about 17000 cm², between about 500 cm² to about 16000 cm², between about 500 cm² to about 15000 cm², between about 500 cm² to about 14000 cm², between about 500 cm² to about 13000 cm², between about 500 cm² to about 12000 cm², between about 500 cm² to about 11000 cm², between about 500 cm² to about 10000 cm², between about 500 cm² to about 9000 cm², between about 500 cm² to about 8000 cm², between about 500 cm² to about 7000 cm², between about 500 cm² to about 6000 cm², between about 500 cm² to about 5000 cm², between about 500 cm² to about 4000 cm², between about 500 cm² to about 3000 cm², between about 500 cm² to about 2000 cm², between about 500 cm² to about 1000 cm², between about 1000 cm² to about 25000 cm², between about 1000 cm² to about 24000 cm², between about 1000 cm² to about 23000 cm², between about 1000 cm² to about 22000 cm², between about 1000 cm² to about 21000 cm², between about 1000 cm² to about 20000 cm², between about 1000 cm² to about 19000 cm², between about 1000 cm² to about 18000 cm², between about 1000 cm² to about 17000 cm², between about 1000 cm² to about 16000 cm², between about 1000 cm² to about 15000 cm², between about 1000 cm² to about 14000 cm², between about 1000 cm² to about 13000 cm², between about 1000 cm² to about 12000 cm², between about 1000 cm² to about 11000 cm², between about 1000 cm² to about 10000 cm², between about 1000 cm² to about 9000 cm², between about 1000 cm² to about 8000 cm², between about 1000 cm² to about 7000 cm², between about 1000 cm² to about 6000 cm², between about 1000 cm² to about 5000 cm², between about 1000 cm² to about 4000 cm², between about 1000 cm² to about 3000 cm², between about 1000 cm² to about 2000 cm², between about 5000 cm² to about 25000 cm², between about 5000 cm² to about 24000 cm², between about 5000 cm² to about 23000 cm², between about 5000 cm² to about 22000 cm², between about 5000 cm² to about 21000 cm², between about 5000 cm² to about 20000 cm², between about 5000 cm² to about 19000 cm², between about 5000 cm² to about 18000 cm², between about 5000 cm² to about 17000 cm², between about 5000 cm² to about 16000 cm², between about 5000 cm² to about 15000 cm², between about 5000 cm² to about 14000 cm², between about 5000 cm² to about 13000 cm², between about 5000 cm² to about 12000 cm², between about 5000 cm² to about 11000 cm², between about 5000 cm² to about 10000 cm², between about 5000 cm² to about 9000 cm², between about 5000 cm² to about 8000 cm², between about 5000 cm² to about 7000 cm², between about 5000 cm² to about 6000 cm², between about 10000 cm² to about 25000 cm², between about 10000 cm² to about 24000 cm², between about 10000 cm² to about 23000 cm², between about 10000 cm² to about 2200 cm², between about 10000 cm² to about 21000 cm², between about 10000 cm² to about 20000 cm², between about 10000 cm² to about 19000 cm², between about 10000 cm² to about 18000 cm², between about 10000 cm² to about 17000 cm², between about 10000 cm² to about 16000 cm², between about 10000 cm² to about 15000 cm², between about 10000 cm² to about 14000 cm², between about 10000 cm² to about 13000 cm², between about 10000 cm² to about 12000 cm², between about 10000 cm² to about 11000 cm², between about 15000 cm² to about 25000 cm², between about 15000 cm² to about 24000 cm², between about 15000 cm² to about 23000 cm², between about 15000 cm² to about 22000 cm², between about 15000 cm² to about 21000 cm², between about 15000 cm² to about 20000 cm², between about 15000 cm² to about 19000 cm², between about 15000 cm² to about 18000 cm², between about 15000 cm² to about 17000 cm², between about 15000 cm² to about 16000 cm², between about 20000 cm² to about 25000 cm², between about 20000 cm² to about 24000 cm², between about 20000 cm² to about 23000 cm², between about 20000 cm² to about 22000 cm², between about 20000 cm² to about 21000 cm², or about 25 cm². In some examples, two or more depth filters are fluidly connected to a manifold in order to increase the amount of recombinant protein flowed through a depth filter at one or more steps in a purification process.

The step of flowing the recombinant protein through a depth filter can result in substantially complete removal of soluble protein aggregates. For example, the step of flowing the recombinant protein through a depth filter can provide a filtrate that includes the purified recombinant protein and is substantially free (such as about or at least 90% free, about or at least 90.5% free, about or at least 91.0% free, about or at least 91.5% free, about or at least 92.0% free, about or at least 92.5% free, about or at least 93.0% free, about or at least 93.5% free, about or at least 94.0%, about or at least 94.5% free, about or at least 95.0% free, about or at least 95.5% free, about or at least 96.0% free, about or at least 96.5% free, about or at least 97.0% free, about or at least 97.5% free, about or at least 98.0% free, about or at least 98.5% free, about or at least 99.0% free, about or at least 99.5% free, or about or at least 99.8% free). In some embodiments, the depth filter provides a filtrate that includes the purified recombinant protein and no detectable soluble protein aggregates.

Methods for detecting the level or amount of protein aggregates are known in the art. For example, size exclusion chromatography, native (non-denaturing) gel chromatography, analytical ultracentrifugation (AUC), field-flow fractionation (FFF), and dynamic light scattering (DLS) can be used to detect the amount of soluble protein aggregates are present in the depth filter filtrate.

In one embodiment of the methods, a constant pressure mode of filtration or a constant flow mode of operation is used. A protein solution can be retained by a pressurized reservoir and pumped through a depth filter by the pressure in the reservoir. The solution is subjected to a normal flow mode of filtration with the aggregates being retained by the depth filter and an aggregate-free solution is discharged as the filtrate. The filtrate can be passed through a conduit for downstream processing, such as one or more unit operations. By operating in this manner, soluble protein aggregates are retained by the depth filter. Alternatively, a pump located between the reservoir and the depth filter could be used to create constant pressure and maintain constant flow through the depth filter. The protein solution is subjected to a normal flow mode of filtration with the aggregates being retained by the depth filter and an aggregate-free solution discharged as the filtrate from the depth filter. The filtrate can be passed through a conduit for further downstream processing, such as the filtrate can be flowed through one or more additional depth filters, a virus filter, and/or one or more unit operations can be performed on the purified recombinant protein.

Non-limiting depth filters that can be used to remove aggregates are described herein and additional depth filters that can be used are known in the art. Representative suitable depth filters include those formed from fibrous media formed of silica, cellulosic fibers, synthetic fibers or blends thereof, such as CUNO® Zeta PLUS® Delipid filters (3M, St. Paul, Minn.), CUNO® Emphaze AEX filters (3M, St. Paul, Minn.), CUNO® 90ZA08A filters (3M, St. Paul, Minn.), CUNO®DELI08A Delipid filters (3M, St. Paul, Minn.), Millipore XOHC filters (EMD Millipore, Billerica, Mass.), MILLISTAK® pads (EMD Millipore, Billerica, Mass.), microporous membranes that are either charged or have a surface chemistry (such as hydrophilicity or hydrophobicity, or a positive or negative charge as are taught by U.S. Pat. Nos. 5,629,084 and 4,618,533) made from a material selected from the group consisting of regenerated cellulose, polyethersulfone, polyarylsulphone, polysulfone, polyimide, polyamide or polyvinylidenedifluoride (PVDF), such as charged DURAPORE® membrane, hydrophobic DURAPORE® membrane, hydrophobic AERVENT® membrane and INTERCEPT™ Q quaternary charged membrane, all available from EMD Millipore, Billerica, Mass.

### One or More Unit Operations

Some embodiments of any of the methods described herein include, between the step of capturing and the step of flowing the recombinant protein through a depth filter, the step of performing one or more (e.g., two, three, four, or five) unit operations on the solution including the recombinant protein, e.g., one or more unit operations selected from the group of filtering (e.g., ultrafiltration/diafiltration to concentrate the recombinant protein in a solution), purifying the recombinant protein, polishing the recombinant protein, viral inactivation, removing viruses by filtration, and adjusting one or both of the pH and ionic concentration of the solution comprising the recombinant protein. Some embodiments of any of the methods described herein include, between the step of capturing and the step of flowing the recombinant protein through a depth filter, the step of performing one or more (e.g., two, three, four, or five) unit operations on the solution including the recombinant protein, e.g., one or more unit operations from the group of ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein. In some embodiments of any of the methods described herein, the methods include between the capturing step and the step of flowing the recombinant protein through a depth filter, performing the sequential unit operations of polishing (e.g., by performing hydrophobic interaction chromatography) and ultrafiltration/diafiltration to concentrate the recombinant protein in a solution.

Some embodiments of any of the methods described herein further include performing one or more (e.g., two, three, four, or five) unit operations before the capturing step, e.g., one or more unit operations selected from the group of clarifying a culture medium, filtration (e.g., ultrafiltration/diafiltration to concentrate the recombinant protein in a solution), viral inactivation, viral filtration, purifying, and adjusting one or both of the pH and ionic concentration of a solution comprising the recombinant protein. Some embodiments of any of the methods described herein further include performing one or more (e.g., two, three, four, or five) unit operations before the capturing step, e.g., one or more unit operations selected from the group of ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein. In some embodiments, the methods further include, prior to the capturing step, the sequential steps of clarification of culture media, ultrafiltration/diafiltration to concentrate the recombinant protein, and viral inactivation.

Some embodiments further include performing one or more unit operations after the step of flowing the recombinant protein through a depth filter, e.g., one or more unit operations selected from the group of purifying the recombinant protein, polishing the recombinant protein, inactivating viruses, filtration, removing viruses by filtration (viral filtration), adjusting one or both of the pH and ionic concentration of a solution comprising the purified recombinant protein, or passing the fluid through an additional depth filter. In some embodiments of any of the methods described herein, the unit operation of viral filtration occurs immediately following the step of flowing the recombinant protein through the depth filter. Some embodiments of any of the methods described herein include performing, after the step of flowing the recombinant protein through a depth filter, the unit operations of, e.g., purifying the recombinant protein and performing viral filtration. Some embodiments of any of the methods described herein include performing, after the step of flowing the recombinant protein through a depth filter, the unit operations of, e.g., polishing the recombinant protein and performing viral filtration. Some embodiments of any of the methods described herein include performing, after the step of flowing the recombinant protein through a depth filter, the unit operations of, e.g., purifying the recombinant protein (e.g., through cation exchange chromatography), polishing the recombinant protein (e.g., through anion exchange chromatography), and performing viral filtration. Some embodiments of any of the methods described herein include performing, after the step of flowing the recombinant protein through a depth filter, the unit operations of, e.g., ultrafiltration/diafiltration, purifying the recombinant protein (e.g., through cation exchange chromatography), polishing the recombinant protein (e.g., through anion exchange chromatography), and performing viral filtration.

### Purifying and Polishing the Recombinant Protein

The methods described herein can include a step of purifying the recombinant protein using at least one chromatography column that can be used to perform the unit operation of purifying a recombinant protein. The methods described herein can include a step of polishing the recombinant protein using at least one chromatography column or chromatographic membrane that can be used to perform the unit operation of polishing the recombinant protein.

The at least one chromatography column for purifying the recombinant protein can include a resin that utilizes a capture mechanism (such as any of the capture mechanisms described herein or known in the art), or a resin that can be used to perform anion exchange, cation exchange, or molecular sieve chromatography. The at least one chromatography column or chromatographic membrane for polishing the recombinant protein can include a resin can be used to perform anion exchange, cation exchange, or molecular sieve chromatography (such as any of the exemplary resins for performing anion exchange, cation exchange, or molecular sieve chromatography known in the art).

The size, shape, and volume of the at least one chromatography column for purifying the recombinant protein, and/or the size and shape of the at least one chromatography column or chromatographic membrane for polishing the recombinant protein can any of combination of the exemplary sizes, shapes, and volumes of chromatography columns or chromatographic membranes described herein or known in the art. Purifying or polishing a recombinant protein can, e.g., include the steps of loading, washing, eluting, and equilibrating the at least one chromatography column or chromatographic membrane used to perform the unit of operation of purifying or polishing the recombinant protein. Typically, the elution buffer coming out of a chromatography column or chromatographic membrane used for purifying comprises the recombinant protein. Typically, the loading and/or wash buffer coming out of a chromatography column or chromatographic membrane used for polishing comprises the recombinant protein.

For example, the size of the chromatography column for purifying the recombinant protein can have a volume of, e.g., between about 1.0 mL to about 650 L (e.g., between about 5.0 mL and about 600 L, between about 5.0 mL and about 550 L, between about 5.0 mL and about 500 L, between about 5.0 mL and about 450 L, between about 5.0 mL and about 400 L, between about 5.0 mL and about 350 L, between about 5.0 mL and about 300 L, between about 5.0 mL and about 250 L, between about 5.0 mL and about 200 L, between about 5.0 mL and about 150 L, between about 5.0 mL and about 100 L, between about 5.0 mL and about 50 L, between about 5.0 mL and about 10 L, between about 5.0 mL and about 1.0 L, between about 5.0 mL to about 900 mL, between about 5.0 mL to about 800 mL, between about 5.0 mL to about 700 mL, between about 5.0 mL to about 600 mL, between about 5.0 mL to about 500 mL, between about 5.0 mL to about 400 mL, between about 5.0 mL to about 300 mL, between about 5.0 mL to about 200 mL, between about 5.0 mL to about 180 mL, between about 5.0 mL to about 160 mL, between about 5.0 mL to about 140 mL, between about 5.0 mL to about 120 mL, between about 5.0 mL to about 100 mL, between about 5.0 mL to about 80 mL, between about 5.0 mL to about 60 mL, between about 5.0 mL to about 40 mL, between about 5.0 mL to about 30 mL, or between about 5.0 mL to about 25 mL).

The linear flow rate of the fluid comprising the recombinant protein as it is loaded onto the at least one chromatography column for purifying the recombinant protein can be, e.g., between 50 cm/hour to about 600 cm/hour, between about 50 cm/hour to about 550 cm/hour, between about 50 cm/hour to about 500 cm/hour, between about 50 cm/hour to about 450 cm/hour, between about 50 cm/hour to about 400 cm/hour, between about 50 cm/hour to about 350 cm/hour, between about 50 cm/hour to about 300 cm/hour, between about 50 cm/hour to about 250 cm/hour, between about 50 cm/hour to about 200 cm/hour, between about 50 cm/hour to about 150 cm/hour, or between about 50 cm/hour to about 100 cm/hour (e.g., for a chromatography column have a diameter of between about 100 cm to about 200 cm). The concentration of the recombinant protein loaded onto the chromatography column for purifying the recombinant protein can be, e.g., between about 0.05 mg/mL to about 90 mg/mL recombinant protein (e.g., between about 0.1 mg/mL to about 90 mg/mL, between about 0.1 mg/mL to about 80 mg/mL, between about 0.1 mg/mL to about 70 mg/mL, between about 0.1 mg/mL to about 60 mg/mL, between about 0.1 mg/mL to about 50 mg/mL, between about 0.1 mg/mL to about 40 mg/mL, between about 0.1 mg/mL to about 30 mg/mL, between about 0.1 mg/mL to about 20 mg/mL, between 0.5 mg/mL to about 20 mg/mL, between about 0.1 mg/mL to about 15 mg/mL, between about 0.5 mg/mL to about 15 mg/mL, between about 0.1 mg/mL to about 10 mg/mL, or between about 0.5 mg/mL to about 10 mg/mL recombinant protein). The resin in the at least one chromatography column for purifying can be an anion exchange or cation exchange chromatography resin. The resin in the at least one chromatography column or chromatographic membrane that is used to perform the unit operation of purifying can be a cationic exchange resin.

Following the loading of the recombinant protein, the at least one chromatographic column or chromatographic membrane is washed with at least one washing buffer. As can be appreciated in the art, the at least one (e.g., two, three, or four) washing buffer is meant to elute all proteins that are not the recombinant protein from the at least one chromatography column, while not disturbing the interaction of the recombinant protein with the resin or otherwise eluting the recombinant protein.

The wash buffer can be passed through the at least one chromatography column at a linear flow rate of, e.g., between 50 cm/hour to about 600 cm/hour, between about 50 cm/hour to about 550 cm/hour, between about 50 cm/hour to about 500 cm/hour, between about 50 cm/hour to about 450 cm/hour, between about 50 cm/hour to about 400 cm/hour, between about 50 cm/hour to about 350 cm/hour, between about 50 cm/hour to about 300 cm/hour, between about 50 cm/hour to about 250 cm/hour, between about 50 cm/hour to about 200 cm/hour, between about 50 cm/hour to about 150 cm/hour, or between about 50 cm/hour to about 100 cm/hour (e.g., for a chromatography column have a diameter of between about 100 cm to about 200 cm). The volume of wash buffer used (such as the combined total volume of wash buffer used when more than one wash buffer is used) can be between about 1× column volume (CV) to about 10×CV, between about 1×CV to about 9×CV, about 1×CV to about 8×CV, about 1×CV to about 7×CV, about 1×CV to about 6×CV, about 2×CV to about 10×CV, about 3×CV to about 10×CV, about 4×CV to about 10×CV, about 2.5×CV to about 5.0×CV, about 5×CV to about 10×CV, or about 5×CV to about 8×CV). The total time of the washing can be between about 2 minutes to about 5 hours (e.g., between about 5 minutes to about 4.5 hours, between about 5 minutes to about 4.0 hours, between about 5 minutes and about 3.5 hours, between about 5 minutes and about 3.0 hours, between about 5 minutes and about 2.5 hours, between about 5 minutes and about 2.0 hours, between about 5 minutes to about 1.5 hours, between about 10 minutes to about 1.5 hours, between about 10 minutes to about 1.25 hours, between about 20 minutes to about 1.25 hours, between about 30 minutes to about 1 hour, between about 2 minutes and 10 minutes, between about 2 minutes and 15 minutes, or between about 2 minutes and 30 minutes).

Following washing of the at least one chromatographic column for purifying the recombinant protein, the recombinant protein is eluted by passing an elution buffer through the column. The elution buffer can be passed through the column that can be used to perform the unit operation of purifying the recombinant protein at a liner flow rate of, e.g., between about 25 cm/hour to about 600 cm/hour, between about 25 cm/hour to about 550 cm/hour, between about 25 cm/hour to about 500 cm/hour, between about 25 cm/hour to about 450 cm/hour, between about 25 cm/hour to about 400 cm/hour, between about 25 cm/hour to about 350 cm/hour, between about 25 cm/hour to about 300 cm/hour, between about 25 cm/hour to about 250 cm/hour, between about 25 cm/hour to about 200 cm/hour, between about 25 cm/hour to about 150 cm/hour, or between about 25 cm/hour to about 100 cm/hour (e.g., for a chromatography column have a diameter of between about 100 cm to about 200 cm). The volume of elution buffer used to elute the recombinant protein from each the at least one chromatographic column for purifying the recombinant protein can be between about 1× column volume (CV) to about 10×CV, between about 1×CV to about 9×CV, between about 1×CV and about 8×CV, between about 1×CV to about 7×CV, about 1×CV to about 6×CV, about 1×CV to about 5×CV, about 1×CV to about 4×CV, about 2×CV to about 10×CV, about 3×CV to about 10×CV, about 4×CV to about 10×CV, about 5×CV to about 10×CV, or about 5×CV to about 9×CV. The total time of the eluting can be between about 5 minutes to about 3 hours, between about 5 minutes to about 2.5 hours, between about 5 minutes to about 2.0 hours, between about 5 minutes to about 1.5 hours, between about 5 minutes to about 1.5 hours, between about 5 minutes to about 1.25 hours, between about 5 minutes to about 1.25 hours, between about 5 minutes to about 1 hour, between about 5 minutes and about 40 minutes, between about 10 minutes and about 40 minutes, between about 20 minutes and about 40 minutes, or between about 30 minutes and 1.0 hour. Non-limiting examples of elution buffers that can be used in these methods will depend on the resin and/or the therapeutic protein. For example, an elution buffer can include a different concentration of salt (e.g., increased salt concentration), a different pH (e.g., an increased or decreased salt concentration), or a molecule that will compete with the recombinant protein for binding to the resin. Examples of such elution buffers for each of the exemplary capture mechanisms described herein are well known in the art.

Following the elution, and before the next volume of fluid including a recombinant protein can be loaded onto the at least one chromatographic column, the at least one chromatography column or chromatographic membrane must be equilibrated using a regeneration buffer. The regeneration buffer can be passed through the chromatography column at a linear flow rate of, e.g., between about 25 cm/hour to about 600 cm/hour, between about 25 cm/hour to about 550 cm/hour, between about 25 cm/hour to about 500 cm/hour, between about 25 cm/hour to about 450 cm/hour, between about 25 cm/hour to about 400 cm/hour, between about 25 cm/hour to about 350 cm/hour, between about 25 cm/hour to about 300 cm/hour, between about 25 cm/hour to about 250 cm/hour, between about 25 cm/hour to about 200 cm/hour, between about 25 cm/hour to about 150 cm/hour, or between about 25 cm/hour to about 100 cm/hour (e.g., for a chromatography column have a diameter of between about 100 cm to about 200 cm). The volume of regeneration buffer used for equilibration can be, e.g., between about 1× column volume (CV) to about 10×CV, between about 1×CV to about 9×CV, between about 1×CV to about 8×CV, between about 1×CV to about 7×CV, between about 1×CV to about 6×CV, between about 2×CV to about 10×CV, between about 3×CV to about 10×CV, between about 2×CV to about 5×CV, between about 2.5×CV to about 7.5×CV, between about 4×CV to about 10×CV, between about 5×CV to about 10×CV, or between about 5×CV to about 10×CV. The concentration of recombinant protein in a solution used to perform the unit operation of purifying the recombinant protein can be between about 0.05 mg/mL to about 90 mg/mL, between about 0.1 mg/mL to about 90 mg/mL, between about 0.1 mg/mL to about 80 mg/mL, between about 0.1 mg/mL to about 70 mg/mL, between about 0.1 mg/mL to about 60 mg/mL, between about 0.1 mg/mL to about 50 mg/mL, between about 0.1 mg/mL to about 40 mg/mL, between about 2.5 mg/mL and about 7.5 mg/mL, between about 0.1 mg/mL to about 30 mg/mL, between about 0.1 mg/mL to about 20 mg/mL, between 0.5 mg/mL to about 20 mg/mL, between about 0.1 mg/mL to about 15 mg/mL, between about 0.5 mg/mL to about 15 mg/mL, between about 0.1 mg/mL to about 10 mg/mL, or between about 0.5 mg/mL to about 10 mg/mL recombinant protein.

The at least one chromatography column or chromatography membrane that can be used to perform the unit operation of polishing the recombinant protein can include a resin that can be used to perform cation exchange, anion exchange, hydrophobic, mixed-mode, or molecular sieve chromatography. As can be appreciated in the art, polishing can include the steps of loading, chasing, and regenerating the chromatography column or chromatographic membrane. For example, when the steps of loading, chasing, and regenerating are used to perform the polishing, the recombinant protein does not bind the resin in the at least one chromatography column or chromatography membrane, and the recombinant protein is eluted from the chromatography column or chromatographic membrane in the loading and chasing steps, and the regenerating step is used to remove any impurities from the chromatography column or chromatographic membrane. Exemplary linear flow rates and buffer volumes to be used in each of the loading, chasing, and regenerating steps are described below.

The size, shape, and volume of the chromatography column or chromatography membrane for polishing the recombinant protein can any of combination of the exemplary sizes, shapes, and volumes of chromatography columns or chromatographic membranes described herein. For example, the size of the at least one chromatography column or chromatographic membrane can have a volume between about 2.0 mL to about 650 L, between about 2.0 mL and about 600 L, between about 2.0 mL and about 550 L, between about 2.0 mL and about 500 L, between about 2.0 mL and about 450 L, between about 2.0 mL and about 400 L, between about 2.0 mL and about 350 L, between about 2.0 mL and about 300 L, between about 2.0 mL and about 250 L, between about 2.0 mL and about 200 L, between about 2.0 mL and about 150 L, between about 2.0 mL and about 100 L, between about 2.0 mL and about 50 L, between about 2.0 mL and about 25 L, between about 2.0 mL and about 10 L, between about 2.0 L and about 5 L, between about 2.0 mL and about 2 L, between about 2.0 mL and about 1 L, between about 2.0 mL and about 800 mL, between about 2.0 mL and about 600 mL, between about 2.0 mL and about 400 mL, between about 2.0 mL and about 200 mL, between about 2.0 mL to about 180 mL, between about 2.0 mL to about 160 mL, between about 2.0 mL to about 140 mL, between about 2.0 mL to about 120 mL, between about 2.0 mL to about 100 mL, between about 2.0 mL to about 80 mL, between about 2.0 mL to about 60 mL, between about 2.0 mL to about 40 mL, between about 2.0 mL to about 40 mL, between about 2.0 mL to about 30 mL, between about 5.0 mL to about 30 mL, between about 2.0 mL to about 25 mL, between about 2.0 mL to about 10 mL, or between about 2.0 mL to about 5 mL. The at least one chromatography column can also be described in terms of its diameter. For example, the at least one chromatography column provided herein can have a diameter of between about 1 cm to about 200 cm, between about 1 cm to about 180 cm, between about 1 cm and about 160 cm, between about 1 cm and about 140 cm, between about 1 cm and about 120 cm, between about 1 cm and about 100 cm, between about 1 cm and about 80 cm, between about 1 cm and about 60 cm, between about 1 cm and about 40 cm, between about 1 cm and about 20 cm, or between about 1 cm and about 10 cm. The linear flow rate of the fluid comprising the recombinant protein as it is loaded onto the chromatography column or chromatographic membrane can be between about 25 cm/hour to about 600 cm/hour, between about 25 cm/hour to about 550 cm/hour, between about 25 cm/hour to about 500 cm/hour, between about 25 cm/hour to about 450 cm/hour, between about 25 cm/hour to about 400 cm/hour, between about 25 cm/hour to about 350 cm/hour, between about 25 cm/hour to about 300 cm/hour, between about 25 cm/hour to about 250 cm/hour, between about 25 cm/hour to about 200 cm/hour, between about 25 cm/hour to about 150 cm/hour, or between about 25 cm/hour to about 100 cm/hour (e.g., for a chromatography column have a diameter of between about 100 cm to about 200 cm). The amount of recombinant protein loaded per mL of resin can be between about 5 mg/mL to about 250 mg/mL, between about 5 mg/mL to about 200 mg/mL, between about 5 mg/mL to about 150 mg/mL, between about 5 mg/mL to about 100 mg/mL, between about 5 mg/mL to about 80 mg/mL, between about 5 mg/mL to about 60 mg/mL, between about 5 mg/mL to about 40 mg/mL, between about 5 mg/mL to about 20 mg/mL, between about 5 mg/mL to about 15 mg/mL, or between about 5 mg/mL to about 10 mg/mL. The resin in the chromatography column or chromatographic membrane for polishing can be an anion exchange or cation exchange resin. The resin can be, e.g., a cationic exchange resin.

Following the loading step, a chasing step is performed. For example, a chase buffer can be passed through the at least one chromatography membrane or chromatographic membrane to collect the recombinant protein that does not substantially bind to the column or membrane). In these examples, the chase buffer can be passed through the column or membrane at a linear flow rate of between about 25 cm/hour to about 600 cm/hour, between about 25 cm/hour to about 550 cm/hour, between about 25 cm/hour to about 500 cm/hour, between about 25 cm/hour to about 450 cm/hour, between about 25 cm/hour to about 400 cm/hour, between about 25 cm/hour to about 350 cm/hour, between about 25 cm/hour to about 300 cm/hour, between about 25 cm/hour to about 250 cm/hour, between about 25 cm/hour to about 200 cm/hour, between about 25 cm/hour to about 150 cm/hour, or between about 25 cm/hour to about 100 cm/hour (e.g., for a chromatography column have a diameter of between about 100 cm to about 200 cm). The volume of chase buffer used can be between about 1× column volume (CV) to about 20×CV, between about between about 1×CV to about 15×CV, between about 5×CV to about 20×CV, between about 1×CV to about 14×CV, about 1×CV to about 13×CV, about 1×CV to about 12×CV, about 1×CV to about 11×CV, about 2×CV to about 11×CV, about 3×CV to about 11×CV, about 4×CV to about 11×CV, about 2.5×CV to about 5.0×CV, about 5×CV to about 11×CV, or about 5×CV to about 10×CV. The total time of the chasing can be between about 2 minutes to about 3 hours, between about 2 minutes to about 2.5 hours, between about 2 minutes to about 2.0 hours, between about 2 minutes to about 1.5 hours, between about 2 minutes to about 1.25 hours, between about 2 minute to about 5 minutes, between about 2 minute to about 10 minutes, between about 2 minutes to about 4 minutes, between about 30 minutes to about 1 hour, between about 2 minutes and 15 minutes, or between about 2 minutes and 30 minutes. The combined concentration of recombinant protein present in the filtrate coming through the column in the loading step and the chasing step can be between about 0.1 mg/mL to about 250 mg/mL recombinant protein, between about 0.1 mg/mL to about 200 mg/mL recombinant protein, between about 0.1 mg/mL to about 150 mg/mL recombinant protein, between about 0.1 mg/mL to about 100 mg/mL recombinant protein, between about 0.1 mg/mL to about 80 mg/mL recombinant protein, between about 0.1 mg/mL to about 70 mg/mL recombinant protein, between about 0.1 mg/mL to about 60 mg/mL recombinant protein, between about 0.1 mg/mL to about 50 mg/mL recombinant protein, between about 0.1 mg/mL to about 40 mg/mL recombinant protein, between about 2.5 mg/mL and about 7.5 mg/mL recombinant protein, between about 0.1 mg/mL to about 30 mg/mL recombinant protein, between about 0.1 mg/mL to about 20 mg/mL recombinant protein, between 0.5 mg/mL to about 20 mg/mL recombinant protein, between about 0.1 mg/mL to about 15 mg/mL recombinant protein, between about 0.5 mg/mL to about 15 mg/mL recombinant protein, between about 0.1 mg/mL to about 10 mg/mL recombinant protein, between about to 0.5 mg/mL to about 10 mg/mL recombinant protein, or between about 1 mg/mL and about 5 mg/mL recombinant protein.

Following the chasing step and before the next volume of fluid is loaded, the column or membrane must be regenerated using a regeneration buffer. Regeneration buffer can be passed through the column or membrane for polishing at a linear flow rate of between about 25 cm/hour to about 600 cm/hour, between about 25 cm/hour to about 550 cm/hour, between about 25 cm/hour to about 500 cm/hour, between about 25 cm/hour to about 450 cm/hour, between about 25 cm/hour to about 400 cm/hour, between about 25 cm/hour to about 350 cm/hour, between about 25 cm/hour to about 300 cm/hour, between about 25 cm/hour to about 250 cm/hour, between about 25 cm/hour to about 200 cm/hour, between about 25 cm/hour to about 150 cm/hour, or between about 25 cm/hour to about 100 cm/hour. The volume of regeneration buffer used to regenerate can be between about 1× column volume (CV) to about 20×CV, between about 1×CV to about 15×CV, between about 5×CV to about 20×CV, between about 1×CV to about 14×CV, about 1×CV to about 13×CV, about 1×CV to about 12×CV, about 1×CV to about 11×CV, about 2×CV to about 11×CV, about 3×CV to about 11×CV, about 4×CV to about 11×CV, about 2.5×CV to about 5.0×CV, about 5×CV to about 11×CV, or about 5×CV to about 10×CV.

In other examples, the one or more chromatography column(s) and/or chromatographic membranes used to perform the unit operation of polishing include a resin that selectively binds or retains impurities present in a fluid comprising the recombinant protein, and instead of regenerating the one or more column(s) and/or membrane(s), the one or more column(s) and/or membrane(s) are replaced (such as with a similar column or membrane) once the binding capacity of the resin in the one or more column(s) and/or membrane(s) has been reached or is substantially close to being reached.

### Inactivation of Viruses/Viral Filtration

The unit operation of inactivating viruses present in a fluid comprising the recombinant therapeutic protein can be performed using a chromatography column, a chromatography membrane, or a holding tank that is capable of incubating a fluid comprising the recombinant therapeutic protein at a pH of between about 3.0 to 5.0, between about 3.5 to about 4.5, between about 3.5 to about 4.25, between about 3.5 to about 4.0, between about 3.5 to about 3.8, or about 3.75 for a period of at least 25 minutes, a period of between about 30 minutes to 1.5 hours, a period of between about 30 minutes to 1.25 hours, a period of between about 0.75 hours to 1.25 hours, or a period of about 1 hour.

Viruses can be removed by filtration. For example, viral filtration can be performed before and/or after the step of flowing the recombinant protein through a depth filter. Viruses can be removed from a solution comprising recombinant protein by either a normal flow filter (NFF) or a tangential flow filtration (TFF) filter such as is described in U.S. Pat. No. 6,365,395. In either TFF mode or NFF mode, filtration is conducted under conditions to retain the virus, generally having a 20 to 100 nanometer (nm) diameter, on the membrane surface while permitting passage of the recombinant protein through the membrane.

Representative suitable ultrafiltration membranes that can be utilized in the viral filtration step include those formed from regenerated cellulose, polyethersulfone, polyarylsulphones, polysulfone, polyimide, polyamide, polyvinylidenedifluoride (PVDF) or the like and are known as VIRESOLVE® membranes and RETROPORE™ membranes available from EMD Millipore, Billerica, Mass. These can be supplied in either a cartridge (NFF) form, such as VIRESOLVE® NFP viral filters, or as cassettes (for TFF), such as PELLICON® cassettes, available from EMD Millipore, Billerica, Mass.

Some methods described herein can include a step of adjusting the pH and/or ionic concentration of a solution comprising the recombinant protein. As described herein, the pH and/or ionic concentration of a solution comprising the recombinant protein can be adjusted (before and/or after it is fed into a depth filter) by adding a buffer to the solution (e.g., through the use of an in-line buffer adjustment reservoir).

### Formulating the Purified Recombinant Protein

Some embodiments of any of the methods described herein further include a step of formulating the recombinant protein or the recombinant protein product into a pharmaceutical composition. For example, formulating can include adding a pharmaceutically acceptable excipient to the purified recombinant protein or the recombinant protein product (e.g., produced by any of the methods of purifying a recombinant protein or any of the methods of manufacturing a recombinant protein product described herein). Formulating can include mixing a pharmaceutically acceptable excipient with the purified recombinant protein or the recombinant protein product. Examples of pharmaceutically acceptable excipients (e.g., non-naturally occurring pharmaceutically acceptable excipients) are well known in the art. In some embodiments, the purified recombinant protein or the recombinant protein product is formulated for intravenous, intraarterial, subcutaneous, intraperitoneal, or intramuscular administration.

### EXAMPLES

Several general protocols are described below, which may be used in any of the methods described herein and do not limit the scope of the invention described in the claims.

### Example 1

### Soluble Protein Aggregate Levels in a Method of Purifying a Recombinant Antibody

A first set of experiments was performed to evaluate the effect of depth filters for removing soluble protein aggregates in processes for purifying two different recombinant antibodies. In this example each process includes at least the following unit operations: antibody capture using protein A chromatography, viral inactivation, depth filtration, ulftrafiltration/diafiltration, and ion exchange chromatography. The amount of antibody aggregates, monomer (non-aggregated antibody), and host cell protein were measured after each unit operation. For example, the levels of aggregate, monomers, and HCP were determined in the pooled protein A chromatography eluate, the solution after viral inactivation, the depth filter eluate, and the pooled ion exchange chromatography eluate in the processes for purifying two different antibodies (Antibody A and Antibody B).

The data in Tables 1 and 2 show that the use of depth filtration in a recombinant antibody purification process results in a significant decrease in soluble protein aggregates during an antibody purification process (a reduction from 4.5% aggregates to 0.6% aggregates in Table 1, and a reduction from 2.2% aggregates to 0.2% aggregates in Table 2). The process shown in Table 1 consists of protein A capture (ProA) followed by viral inactivation (VI), then depth filtration, ultrafiltration diafiltration (UF/DF1), and ion exchange chromatography (IEX). The solution material in the process shown in Table 1 was analyzed for host cell protein (HCP) concentration, % protein monomer, % protein aggregate, and picograms of DNA/mg protein after each unit operation. The process shown in Table 2 consists of protein A capture (ProA), followed by viral inactivation (VI), then depth filtration, and ultrafiltration diafiltration (UF/DF1). The solution material in the process shown in Table 2 was analyzed for HCP, % protein monomer, % protein aggregate, and picograms of DNA/mg protein after each unit operation.

The data in Tables 1 and 2 demonstrate that inclusion of a depth filtration step early in a process for purifying recombinant antibodies can significantly reduce protein aggregates. The data also demonstrate that, even after the depth filtration step in the antibody purification process, the amount of protein aggregates can again increase with the performance of additional steps.

TABLE 1
Soluble Protein Aggregates in a Process for Purifying a Recombinant
Antibody

| Sample | HCP | Log HCP | % | % | pg |
|---|---|---|---|---|---|
| Description | (ng/mg) | Clearance | Monomer | Aggregate | DNA/mg |
| ProA Pool | | | 96.1 | 3.9 | N/A |
| VI Pool | 6447 | N/A | 95.5 | 4.5 | 3913.5 |
| Depth Filtered | 207 | 1.5 | 99.4 | 0.6 | <LOQ |
| VI Pool | | | | | |
| UF/DF1 Pool | | | 98.6 | 1.4 | N/A |
| IEX Pool | 92 | 0.4 | 99.6 | 0.4 | <LOQ |

TABLE 2
Amount of Soluble Protein Aggregates in Different Steps of a Process for
Purifying Recombinant Protein B

| Sample Description | HCP (ng/mg) | % Monomer | % Aggregate |
|---|---|---|---|
| ProA Pool | | 98.4 | 1.6 |
| VI Pool | 2064.1 | 97.9 | 2.2 |
| Depth Filtered VI Pool | 28 | 99.8 | 0.2 |
| UF/DF1 Pool | N/A | 99.5 | 0.5 |

### Example 2

### Use of Depth Filtration Downstream in a Process for Purifying a Recombinant Fc-Fusion Protein

A set of experiments was performed to determine whether the downstream or later use of a depth filter in a recombinant protein purification process (e.g., after cell culture media clarification, ultrafiltration/diafiltration, viral inactivation, protein A capturing, hydrophobic interaction chromatography (e.g., polishing), and a second ultrafiltration/diafiltration step) would decrease the flux decay in a viral filter in a method for purifying a recombinant Fc-fusion protein from a clarified cell culture.

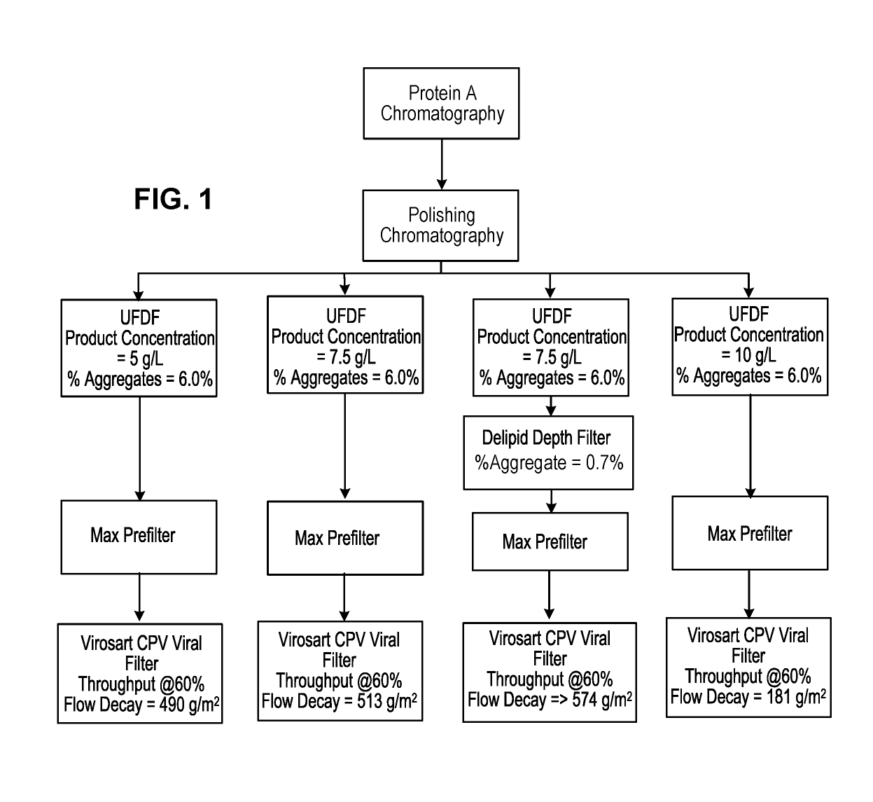

The different purification unit operations are shown in FIG. 1 and included protein A chromatography and hydrophobic interaction chromatography (e.g., polishing) for each process. The processes varied according to the diagram in FIG. 1. One process included concentration to a recombinant protein concentration of 5 mg/mL, a pre-filtration, and viral filtration. One process included concentration to a recombinant protein concentration of 7.5 mg/mL, a pre-filtration, and viral filtration. The next process included concentration to a recombinant protein concentration of 7.5 mg/mL, depth filtration, a pre-filtration, and viral filtration. The fourth process included concentration to a recombinant protein concentration of 10 g/L, a pre-filtration, and viral filtration. Each tested methods included a first ultrafiltration/diafiltration step prior to protein A chromatography. A schematic of the different steps in the different tested purification methods, the percentage of soluble protein aggregates at each step, the viral filter throughput, and the flow decay of the virus filter are shown in FIG. 1. (FIG. 1 does not show the ultrafiltration/diafiltration step that occurs before the protein A chromatography step in each method.) The percentage of soluble protein aggregates at each step, the viral filter throughput, and the flow decay of the virus filter are also shown in FIG. 1. The data in FIG. 1 show that the viral filter throughput (g/m²) in the viral filtration step is increased when the purification method includes a depth filtration step immediately following the second ultrafiltration/diafiltration step and immediately prior to the sequential prefiltration and viral filtration.

A second set of experiments were performed to test whether the use of a depth filter immediately prior to viral filtration in a purification method would result in a reduction in protein aggregates flowing into the virus filter (as compared to a similar method that utilizes a pre-filter rather than a depth filter immediately prior to viral filtration). The purification methods tested in this method include the steps of: clarification of a culture medium, ultrafiltration/diafiltration, virus inactivation, protein A chromatography (e.g., capturing), hydrophobic interaction chromatography (e.g., polishing), ultrafiltration/diafiltration, virus inactivation, protein A chromatography (capturing), hydrophobic interaction chromatography (e.g., polishing), ultrafiltration or diafiltration (e.g., to a concentration of 5 mg/mL, 7.5 mg/mL, or 10 mg/mL), filtration with a Sartorious Max pre-filter or a CUNO Delipid depth filter, and viral filtration.

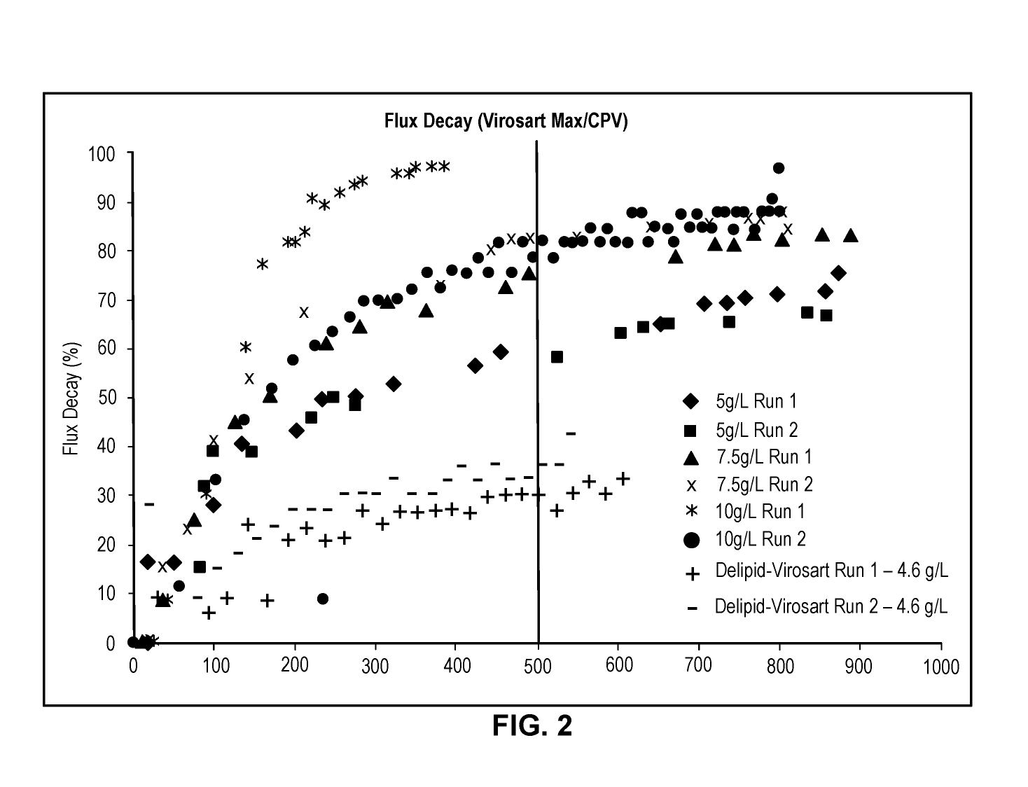

Table 3 shows the percentage of soluble protein aggregates entering into the viral filter (aggregate content in load) and the percentage of soluble protein aggregates present in the pooled virus filter eluate (aggregate content in filtrate pool) for each of the different purification methods tested. Each purification was run in duplicate and shown in Table 3 and FIG. 2. The data show that there was a significantly lower percentage of soluble protein aggregates in the virus filtrate pool from the method that utilized a depth filter immediately prior to viral filtration. See, bottom two rows of Table 3 for CUNO Delipid depth filter. The flux decay of the virus filter as compared to throughput (g/m²) in each of the tested purification methods was plotted and shown in FIG. 2. These data show that there was a lower flux decay at the different throughput values (g/m²) in the purification methods that included the use of a depth filter prior to viral filtration. Compare the flux decay at different throughput values observed for the purification methods that did not include the use of a depth filter prior to viral filtration (see, data for Delipid-Virosart Run 1 and 2-4.6 g/L data).

TABLE 3
Percentage of Soluble Protein Aggregates in Viral Filter Load and
Pooled Virus Filter Eluate in Different Tested Purification Methods

| | | | Aggregate | | | Aggregate |
|---|---|---|---|---|---|---|
| | | Load | content in | | | content in |
| | Prefilter | concentration | load | Load | Filtrate | Filtrate pool |
| Run# | Used | (mg/ml) | (%) | (Particulates/ml) | (Particulates/ml) | (%) |
| 1 | Sartorius | 5 | 6.0 | 5067 | 8571 | 5.9 |
| 2 | Max | 5 | | | | |
| 1 | | 7.5 | | 3684 | 15664 | 6.0 |
| 2 | | 7.5 | | | | |
| 1 | | 10 | | 5056 | 11073 | 5.9 |
| 2 | | 10 | | | | |
| 1 | CUNO | 4.6 | | 5912 | 1374 | 0.7 |
| 2 | Delipid | 4.6 | | | | |

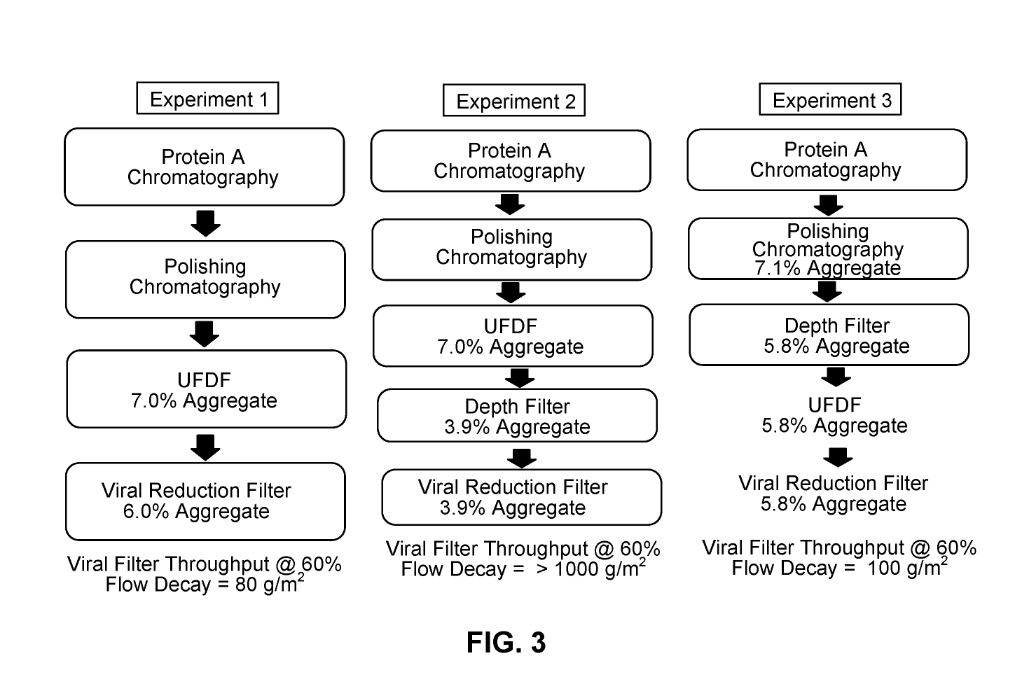

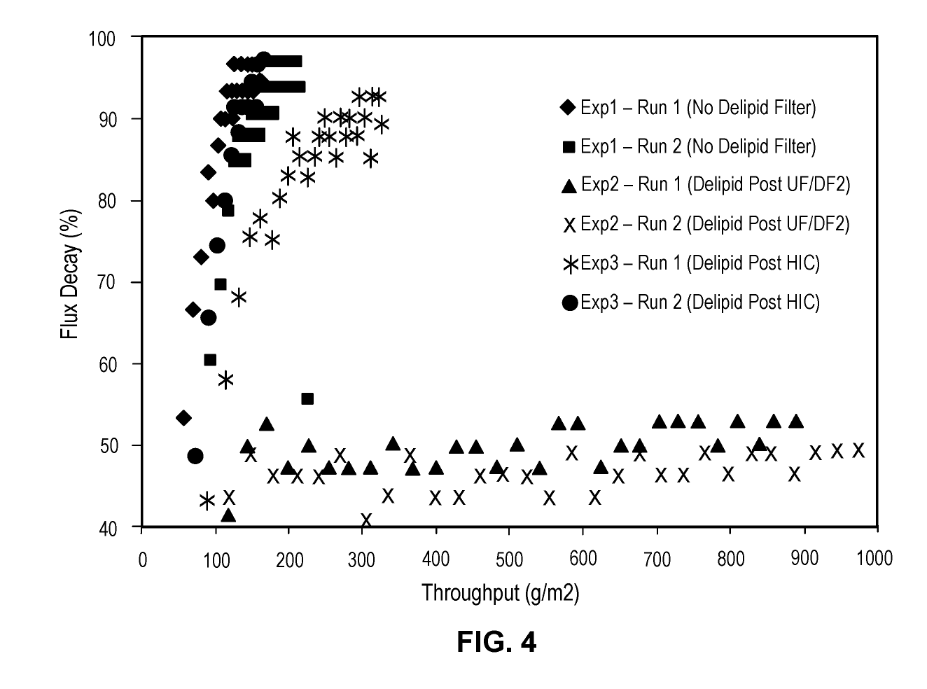

A set of experiments were performed to test the effect of the use of depth filtration at different steps in the process for purifying a recombinant Fc-fusion protein. The different methods included the unit operations of: clarification of culture medium, ultrafiltration/diafiltration, viral inactivation, protein A chromatography (capturing), hydrophobic interaction chromatography, ulfrafiltration/diafiltration, and viral filtration (Experiment 1); clarification of culture medium, ultrafiltration/diafiltration, viral inactivation, protein A chromatography (capturing), hydrophobic interaction chromatography (e.g., polishing), ultrafiltration/diafiltration, depth filtration, and viral filtration (Experiment 2); or clarification of culture medium, ultrafiltration/diafiltration, viral inactivation, protein A chromatography (capturing), hydrophobic interaction chromatography (e.g., polishing), depth filter filtration, ultrafiltration/diafiltration, and viral filtration (Experiment 3). FIG. 3 shows a schematic of the different purification processes, the percentage of soluble protein aggregates at each step and the resulting flow decay of the virus filter. The data showed that the lowest percentage of soluble protein aggregates and the best flow through the viral filter was achieved in the purification method having a step of depth filtration immediately before the step of viral filtration. The data in Table 4 represent the data from similar tested purification processes: with Runs 1 and 2 corresponding to Experiment 1 described above, Runs 3 and 4 corresponding to Experiment 2 above, Runs 5 and 6 corresponding to Experiment 3 above. FIG. 4 shows the flux decay of the virus filter over different throughput values (g/m²) for purification processes corresponding to Experiment 1, Experiment 2, and Experiment 3 processes described above.

The resulting data show that purification methods using a depth filter immediately prior to the viral filtration step resulted in a lower percentage of protein aggregates in the solution that was passed through the viral filter and also resulted in a significant decrease in the flux decay over different throughput values in the virus filter (FIG. 4).

TABLE 4
Soluble Protein Aggregates in Virus Filter Load and Virus Filter Pooled
Eluate in Different Methods of Purifying a Recombinant Fc-Fusion Protein

| | | | Aggregate | Aggregate | | | Aggregate |
|---|---|---|---|---|---|---|---|
| | Placement of | Load | content in | Content in | | | content in |
| | the Delipid | concentration | HIC pool | Viral | Load | Filtrate | Filtrate pool |
| Run# | depth filter | (mg/ml) | (%) | Filter Load | (Particulates/ml) | (Particulates/ml) | (%) |
| 1 | No Delipid | 7.5 | 7.0 | 6.8 | 9959 | 5863 | 6.8 |
| 2 | | 7.5 | | 6.8 | | | 6.8 |
| | Depth filter | | | | | | |
| 3 | Delipid filter | 7.5 | | 3.9 | 691 | 896 | 3.9 |
| 4 | after the | 7.5 | | 3.9 | | | 3.9 |
| | UFDF2 step | | | | | | |
| 5 | Delipid filter | 7.5 | | 5.8 | 1433 | 2890 | 5.8 |
| 6 | after the | 7.5 | | 5.8 | | | 5.7 |
| | HIC step | | | | | | |

### Example 3

### Effect of pH and Filter Load on Depth Filtration

A set of experiment was performed to test the effect of pH and filter load on the depth filtration step performed in a process that included the following steps: clarification of culture medium, ultrafiltration/diafiltration, viral inactivation, protein A chromatography (capturing), hydrophobic interaction chromatography (e.g., polishing), ulfrafiltration/diafiltration, depth filtration, and viral filtration. The solution passed through the depth filter in these methods had a pH of 6.0, 6.5, 7.0, 7.5, 8.0, or 8.5, and optionally, was passed through the filter at a filter load of 70 L/m², 125 L/m², and 180 L/m².

The percentage of soluble protein aggregates in the depth filter loading solution and the depth filter pooled eluates in different processes were measured and are shown in Tables 5 and 6. The data revealed that depth filtration significantly reduced the level of soluble protein aggregates and host cell protein in the purification processes, e.g., using different filter loads and using different recombinant antibody-containing solutions having pH values between 6.0 and 8.5.

In Table 5, the starting aggregate % and HCP (ng/mg) prior to passage through the depth filter was 4.5% aggregates and 3712 ng/mg as shown in the top row. The depth filtration step performed better when the pH was between 6.5 and 7.5. The load also affected depth filter performance, compare pH 6.5 with 180 L/m² and pH 6.5 with 70 L/m². The lower load at pH 6.5 allowed better performance of depth filter removal of both aggregates and HCP.

In Table 6, depth filter performance was evaluated at pH 6.0, pH 7.0, and pH 8.0. The starting HCP (ng/mg) and % aggregate prior to passage through the depth filter was 7,045-9,078 ng/mg and 2.1%-2.8% as shown in the third and sixth columns, respectively. The depth filtration step performed very well at removing HCP at all pH values tested. Aggregate removal was slightly more pH sensitive. See, Table 6.

TABLE 5
Effect of pH and Filter Load on Depth Filtration in
Tested Methods for Purifying a Recombinant Antibody

| | | Filter | | Aggre- | | |
|---|---|---|---|---|---|---|
| | | Load | Yield | gate | HCP | HCP |
| Sample | pH | (L/m2) | (%) | (%) | (ng/mg) | (LRV) |
| Affinity Pool (Depth | | | 99.7% | 4.5 | 3712 | |
| Filter Load) | | | | | | |
| Depth Filter Pool - | 6.5 | 180 | 85.3% | 1.0 | 260 | 1.15 |
| Run 1 | | | | | | |
| Depth Filter Pool - | 6.5 | 70 | 82.9% | 0.4 | 120 | 1.49 |
| Run 2 | | | | | | |
| Depth Filter Pool - | 7.5 | 125 | 86.6% | 1.6 | 218 | 1.23 |
| Run 3 | | | | | | |
| Depth Filter Pool - | 8.5 | 180 | 92.3% | 3.6 | 1085 | 0.53 |
| Run 4 | | | | | | |
| Depth Filter Pool - | 8.5 | 70 | 92.0% | 3.3 | 824 | 0.65 |
| Run 5 | | | | | | |

TABLE 6
Effect of pH on Depth Filtration in Tested Methods for Purifying a
Recombinant Antibody

| | | HCP | Log HCP | % | % |
|---|---|---|---|---|---|
| Sample Description | pH | (ng/mg) | Clearance | Monomer | Aggregate |
| Affinity Pool (Depth Filter Load) | | 7,045 | | 97.5 | 2.5 |
| Run1 | | | | | |
| Depth Filter Pool - Run 1 | 6.0 | 107 | 1.818 | 99.2 | 0.8 |
| Affinity Pool (Depth Filter Load) | | 7,786 | | 97.9 | 2.1 |
| Run 2 | | | | | |
| Depth Filter Pool - Run 2 | 6.0 | 753 | 1.015 | 99.2 | 0.7 |
| Affinity Pool (Depth Filter Load) | | 8,729 | | 97.2 | 2.8 |
| Run1 | | | | | |
| Depth Filter Pool - Run 1 | 7.0 | 2,038 | 0.632 | 98.8 | 1.2 |
| Affinity Pool (Depth Filter Load) | | 8,367 | | 97.7 | 2.3 |
| Run 2 | | | | | |
| Depth Filter Pool - Run 2 | 7.0 | 1,571 | 0.726 | 99.1 | 0.9 |
| Affinity Pool (Depth Filter Load) | | 8,324 | | 97.3 | 2.7 |
| Run1 | | | | | |
| Depth Filter Pool - Run 1 | 8.0 | 2,175 | 0.583 | 98.1 | 1.8 |
| Affinity Pool (Depth Filter Load) | | 9,078 | | 97.6 | 2.4 |
| Run 2 | | | | | |
| Depth Filter Pool - Run 2 | 8.0 | 608 | 1.174 | 98.6 | 1.5 |

### Example 4

### Exemplary Processes that Include Performing Viral Inactivation and Adjusting One or Both of the pH and Ionic Concentration of a Solution Including the Purified Recombinant Protein, Between Capturing and Depth Filtration

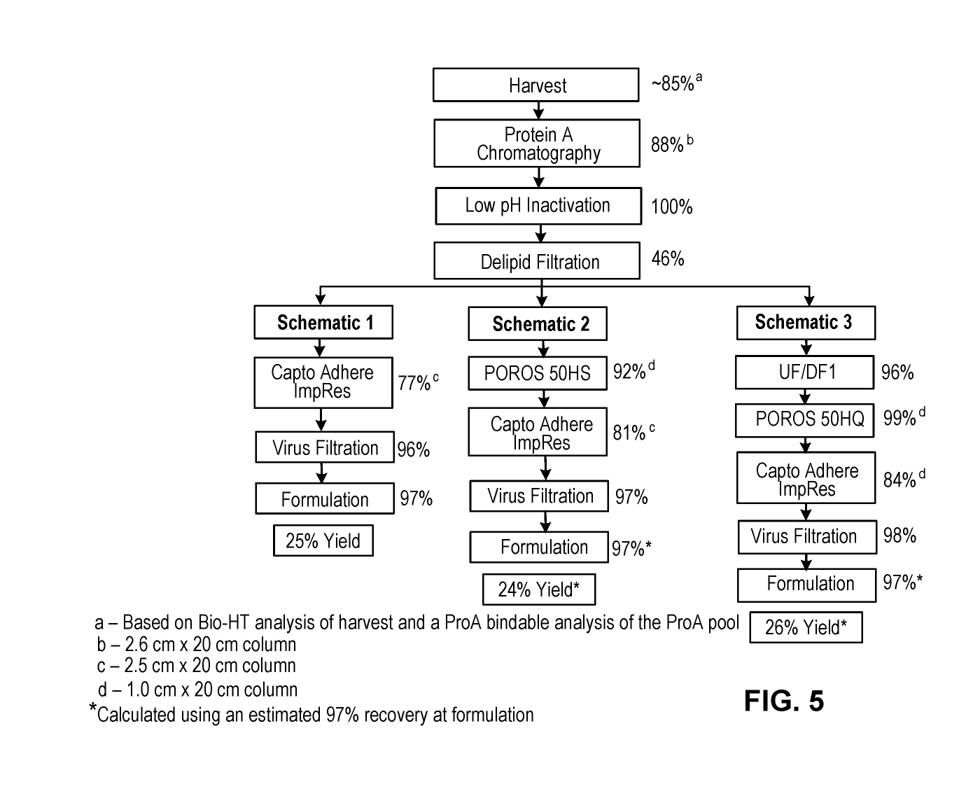

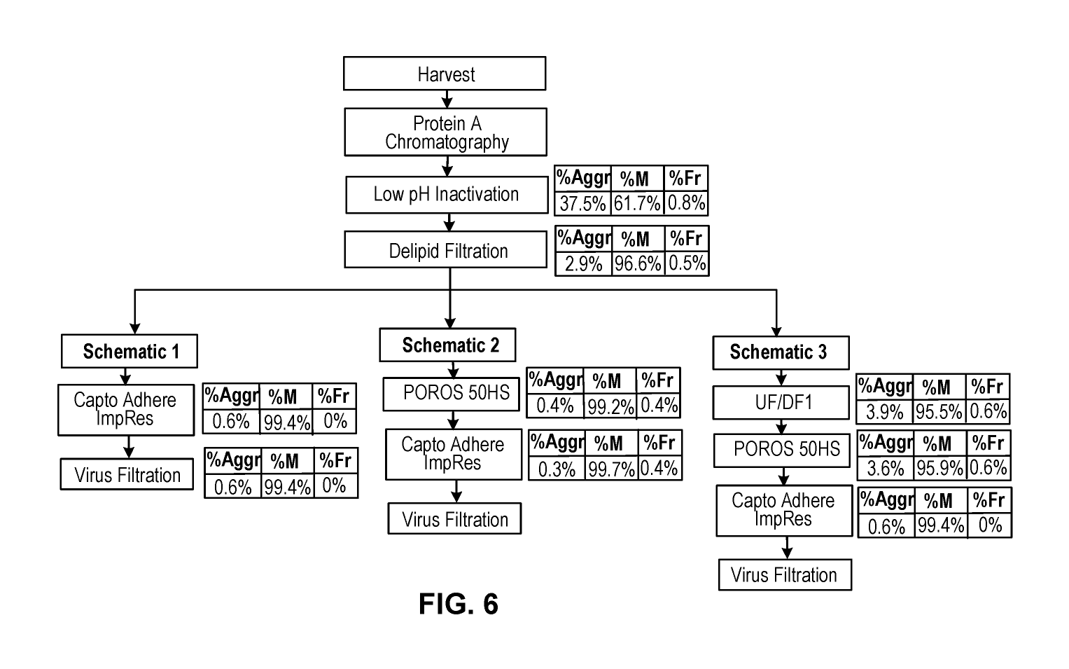

Three different purification processes were tested in this Example. Each tested process included a protein A chromatography capture step and adjustment of one or both of the pH and ionic concentration of a solution including the purified recombinant protein, prior to performing depth filtration and one or more additional downstream unit operations. Each of the tested processes (Schematics 1 to 3) is shown in FIGS. 5 and 6. The product yield and soluble protein aggregate clearance were compared between the three tested processes.

### Materials and Methods

### Materials

Culture medium including eculizumab harvested from a single bioreactor culture run was used in each experiment. The following additional materials were used to perform the tested processes: Millipore DOHC, Millipore A1HC, Millipore Express SHC (0.2 μm), MabSelect™ SuRe™ resin (GE Healthcare), Zeta Plus Dilipid filter (3M DELI08A), Pelicon3 UF/DF membrane, POROS 50HS Column (Life Technologies), POROS 50HQ Resin (Life Technologies), Capto Adhere ImpRes resin (GE Healthcare), Sartorius MAX pre-filter, Sartorius Virosart CPV, AKTA Avant chromatography system (with Unicorn software), a pH meter, and a conductivity meter. All buffers were produced using USP grade chemicals. Table 7 lists the dimensions for each of the columns and filters used in this Example.

TABLE 7
Column and Filter Dimensions

| Column/Filter | Column/Filter Size |
|---|---|
| MabSelect ™ SuRe ™ | 2.6 cm ID × 20 cm bed height (106.1 mL) |
| Delipid Filter | 3 × 25 cm² |
| Pellicon 3 UF/DF | 50 cm², 30 kDa MWCO |
| CA ImpRes | 1 cm ID × 20 cm bed height (15.7 mL) |
| CA ImpRes | 2.5 cm ID × 20 cm bed height (98.1 mL) |
| POROS 50HS | 1.2 cm ID × 20 cm bed height (22.6 mL) |
| POROS 50HQ | 1 cm ID × 20 cm bed height (15.7 mL) |
| Virosart MAX | 5 cm² |
| Virosart CPV | 5 cm² |

### Methods

MabSelect™ SuRe′ chromatography is an Fc affinity-based capture step used to concentrate the product and to remove impurities. The product is bound to the column at neutral pH and is eluted at low pH using 25 mM sodium acetate (pH 3.7). Five chromatography cycles were performed to generate the starting material for the three tested processes (Schematics 1 to 3) depicted in FIGS. 5 and 6. Tables 8 and 9 summarizes the process conditions for protein A purification of eculizumab.

TABLE 8
Protein A Column Specifications and Resin Load Conditions

| Resin type: | MabSelect ™ SuRe ™ |
|---|---|
| Column diameter (cm): | 2.6 |
| Column height estimated (cm): | 20 |
| Column volume (mL): | 106 |
| Load (mg/mL resin): | ~25 |
| Amount loaded × cycle (mg): | ~2450 |

The low pH viral inactivation step was performed to inactivate viruses, e.g., enveloped viruses. The protein A pool was subjected to low pH viral inactivation under the conditions described in Table 10. Material was held at pH 3.70 for 60 minutes at room temperature without stirring after pH adjustment. The material was incubated for 60 minutes and subsequently neutralized to pH 6.0 with 1.0 M Tris base.

Filtration of eculizumab with Zeta Plus Delipid filters (3M DELI08A) was performed under the load conditions described in Table 11. The neutralized low pH viral inactivated pool was filtered through three Zeta Plus Delipid filters in parallel (3×25 cm2 filtration area). Filtration was executed at 150 LMH. A 50 L/m2 recovery flush was executed using 50 mM sodium phosphate, 150 mM sodium chloride, pH 6.0.

TABLE 9
Protein A Chromatography Conditions

| | | | Volume/Collection | | Flow Direction/ |
|---|---|---|---|---|---|
| Step | Block Name | Solution | Criteria | | Flow rate cm/hr |
| 1. | Equilibration | 50 mM Sodium Phosphate, | ≧4.0 | CV | Downflow/300 |
| | | 100 mM Sodium Chloride, | | | |
| | | pH 7.00 | | | |
| 2. | Load | Clarified Harvest | 25 mg/mL resin | | Downflow/300 |
| 3. | Post-load | 50 mM Sodium Phosphate, | ≧4.0 | CV | Downflow/300 |
| | wash 1 | 100 mM Sodium Chloride, | | | |
| | | pH 7.00 | | | |
| 4. | Post-load | 85 mM Sodium Phosphate, | ≧5.0 | CV | Downflow/300 |
| | wash 2 | 100 mM Sodium Chloride, | | | |
| | | 0.7% Caprylic acid, | | | |
| | | 300 mM Arginine, pH 7.5 | | | |
| 5. | Post-load | 50 mM Sodium Phosphate, | ≧4.0 | CV | Downflow/300 |
| | wash 3 | 100 mM Sodium Chloride, | | | |
| | | pH 7.00 | | | |
| 6. | Elution | 25 mM Sodium Acetate, | 100 mAU-100 mAU | | Downflow/300 |
| | | pH 3.70 | (2 mm path-length) | | |
| 7. | Post-elution | 0.1M Citric acid | ≧3.0 | CV | Downflow/300 |
| | Strip | | | | |
| 8. | Post-strip | dH2O | ≧3.0 | CV | Downflow/300 |
| | Flush | | | | |
| 9. | Clean | 0.1N NaOH | 3.0 | CV | Upflow/300 |
| 10. | Static Hold* | 0.1N NaOH | 60 | min | N/A |
| 11. | Pre-Store | 50 mM Sodium Phosphate, | ≧3.0 | CV | 1 CV |
| | Flush | 100 mM Sodium Chloride, | | | Upflow/2 CV |
| | | pH 7.00 | | | Downflow/300 |
| 12. | Storage* | 18% Ethanol | ≧3.0 | CV | Downflow/225 |

TABLE 10
Low pH Viral Inactivation Conditions

| Sub-Step | Parameter | Target/Range |
|---|---|---|
| Acidification | Final Target pH | 3.70 ± 0.10 |
| | Hold Time | 60-70 min |
| Neutralization | Final Target pH | 6.00 ± 0.10 |

TABLE 11
Process Conditions for Delipid Depth Filtration

| Process Parameter | Target/Range |
|---|---|
| Filter Type | 3M DELI08A, Zeta Plus Delipid filter |
| Flush Buffer | 50 mM Sodium phosphate, 100 mM |
| | Sodium chloride pH 6.0 |
| Flush volume (L/m²) | 54 |
| Membrane Surface Area (cm²) | 25 × 3 filters = 75 |
| Load | ≦250 g/m²/≦100 L/m² |
| Post Load Flush (L/m²) | ≦50 L/m² |
| Flow Rate (LMH) | 150 |

POROS 50HS is a flow-through strong cation exchange chromatography step, which is used to provide soluble protein aggregate and impurity clearance. During loading, the antibody flows through the column, while soluble protein aggregates and impurities bind to the column. The impurities are stripped from the column using 2 M sodium chloride. The Delipid filtrate was adjusted to a conductivity of 18 mS/cm using 2 M NaCl and then loaded onto the POROS 50HS column. The process conditions used to perform the depth filtration are shown in Tables 12 and 13.

TABLE 12
Cation Exchange Chromatography Specifications
and Resin Load Conditions

| Process Parameter | Target |
|---|---|
| Resin type: | POROS 50 HS |
| Column diameter (cm): | 1 |
| Column height (cm): | 20 |
| Column volume (mL): | 15.7 |
| Load (mg/mL resin): | 20 |

TABLE 13
Cation Exchange Chromatography Conditions

| | | Volume/Collection | | Flow Direction/ |
|---|---|---|---|---|
| Block Name | Solution | Criteria | | Flow rate cm/hr |
| Pre- | 2M NaCl | ≧2 | CV | Downflow/300 |
| Equilibration | | | | |
| Equilibration | 20 mM Citrate | ≧5 | CV | Downflow/150 |
| | 150 mM NaCl | | | |
| | pH 6.0 | | | |
| Load | Delipid | 20 | mg/mL | Downflow/300 |
| | Filtrate | | | |
| Wash | 20 mM Citrate | ≦10 | CV | Downflow/300 |
| | 150 mM NaCl | | | |
| | pH 6.0 | | | |
| Collection | | Load Collection | | Downflow/300 |
| Criteria | | Criteria >100 mAU | | |
| | | Wash Collection | | |
| | | Criteria | | |
| | | until <100 mAU | | |
| | | (2 mm path-length) | | |
| Strip | 2M NaCl | ≧3 | CV | Upflow/300 |
| Clean | 1.0N NaOH | ≧3 | CV | Upflow/300 |
| Equilibration | 20 mM Citrate | ≧3 | CV | Downflow/300 |
| | 150 mM NaCl | | | |
| | pH 6.0 | | | |
| Storage | 18% Ethanol | ≧3 | CV | Downflow/300 |

Ultrafilration/diafiltration step 1 (UF/DF1) is a concentration and buffer exchange step performed to generate POROS 50HQ load material. Eculizumab was concentrated to 4 mg/mL prior to 6× diafiltration. The UF/DF1 conditions are listed in Table 14.

TABLE 14
UF/DF1 Conditions

| TFF membrane used | Millipore Pellicon 3 | |
|---|---|---|
| Membrane cutoff (Da) | 30,000 | |
| TMP | 1.0-1.5 | bar |
| Feed flow rate | 4-8 | L/m²/min |
| Filter area (m²) | 0.1 | |
| Filter load (g/m²) | ≦300 | |
| Diavolumes | 6X | |
| Target Concentration | 4 | g/L |

| | |
|---|---|
| Diafiltration Buffer | 20 mM Tris, 65 mM NaCl, pH 7.6 |

POROS 50HQ is a flow-through anion exchange chromatography step, which is used to remove impurities and viruses. During loading, the antibody flows through the column and the impurities bind to the column. Following product collection, the impurities are stripped from the column using 2.0 M sodium chloride buffer. Material from the UF/DF1 cycle was processed onto the POROS 50HQ flow-through column using the parameters listed in Tables 15 and 16.

TABLE 15
Anion Exchange Column Specifications
and Resin Load Conditions

| Process Parameter | Target |
|---|---|
| Resin type | POROS 50 HQ |
| Column diameter (cm) | 1 |
| Column height (cm) | 20 |
| Column volume (mL) | 15.7 |
| Load (mg/mL resin) | 25-50 |
| Amount loaded x cycle (mg) | 393-789 |

TABLE 16
Anion Exchange Column Chromatography Conditions

| | | | Volume/Collection | | Flow Direction/ |
|---|---|---|---|---|---|
| Step | Block Name | Solution | Criteria | | Flow rate cm/hr |
| 1. | Pre- | 2M NaCl | ≧2 | CV | Downflow/300 |
| | equilibration | | | | |
| 2. | Equilibration | 20 mM Tris, 65 mM NaCl, | ≧5 | CV | Downflow/300 |
| | | pH 7.6 | | | |
| 3. | Load | UF/DF1 pool | 25-50 mg/mL | | Downflow/300 |
| | | | resin >100 mAU | | |
| | | | (2 mm path-length) | | |
| 4. | Wash | 20 mM Tris, 65 mM NaCl, | <100 | mAU | Downflow/300 |
| | | pH 7.6 | (2 mm path-length) | | |
| 5. | Strip | 2M NaCl | ≧3 | CV | Upflow/300 |
| 6. | Clean | 1.0M NaOH | ≧3 | CV | Upflow/300 |
| 7. | Static Hold | 1.0M NaOH | 45-60 | min | N/A |
| 8. | Flush | 20 mM Tris, 89 mM NaCl, | ≧3 | CV | Downflow/300 |
| | | pH 7.5 | | | |
| 9. | Storage | 18% Ethanol | ≧3 | CV | Downflow/300 |

Capto Adhere ImpRes is a multimodal strong anion exchange chromatography step used in a bind and elute mode to remove impurities. Eculizumab is bound to the column under neutral pH and is eluted by lowering the pH. Delipid filtrate and POROS 50HS pools were adjusted to pH 7.0 with 1.0 M Tris Base and the POROS 50HQ pool was adjusted to pH 7.0 with 1 M citric acid to generate the Capto Adhere ImpRes load material for Schematics 1 to 3, respectively. The column specifications and conditions used to perform the Capto Adhere ImpRes chromatography are shown in Tables 17 and 18.

TABLE 17
Capto Adhere ImpRes Column Specifications
and Resin Load Conditions

| Resin type | Capto Adhere ImpRes |
|---|---|
| Column diameter (cm) | 2.6 |
| Column height estimated (cm) | 20 |
| Column volume (mL) | 106 |
| Load (mg/mL resin) | 20 |
| Amount loaded x cycle (mg) | 2120 |

TABLE 18
Capto Adhere ImpRes Chromatography Process Conditions

| | | | Volume/Collection | | Flow Direction/ |
|---|---|---|---|---|---|
| Step | Block Name | Solution | Criteria | | Flow rate cm/hr |
| 1. | Equilibration | 25 mM Sodium Phosphate, | ≧4.0 | CV | Downflow/225 |
| | | 150 mM Sodium Chloride, | | | |
| | | pH 7.00 | | | |
| 2. | Load | Protein A pool | 20 mg/mL resin | | Downflow/225 |
| 3. | Post-load | 25 mM Sodium Phosphate, | ≧6.0 | CV | Downflow/225 |
| | wash 1 | 150 mM Sodium Chloride, | | | |
| | | pH 7.00 | | | |
| 4. | Elution | 50 mM Sodium Acetate, | 100 mAU-300 mAU | | Downflow/225 |
| | | 150 mM NaCl, pH 5.5 | (2 mm path-length) | | |
| 5. | Post-elution | 0.1M Citric acid | ≧3.0 | CV | Downflow/225 |
| | Strip | | | | |
| 6. | Post Strip | dH₂O | ≧3.0 | CV | Downflow/225 |
| | Flush | | | | |
| 7. | Clean | 0.5N NaOH | 3.0 | CV | Upflow/225 |
| 8. | Static Hold* | 0.5N NaOH | 60 | min | N/A |
| 9. | Pre-Store | 25 mM Sodium Phosphate, | ≧3.0 | CV | 1 CV |
| | Flush | 150 mM Sodium Chloride, | | | Upflow/2 CV |
| | | pH 7.00 | | | Downflow/225 |
| 10. | Storage* | 18% Ethanol | ≧3.0 | CV | Downflow/225 |

The Virosart CPV virus filtration step removes potential viruses that are ≧20 nm. Material from the three test processes (Schematics 1 to 3) was processed through the Sartorius MAX prefilter and Virosart CPV viral filter at a pressure of <30 psi for filter sizing analysis. The Sartorius MAX pre-filtration and Virosart CPV virus filtration step was performed under the conditions listed in Table 19.

TABLE 19
Viral Filtration Conditions

| Process Parameter | Target/Range |
|---|---|
| Prefilter | Sartorius Max, 5 cm² |
| Virus Filter | Virosart CPV, 5 cm² |
| Inlet pressure maximum (psi) | 30 |

### Results

The protein A capture chromatography data are shown in Table 20. Protein A chromatography capture performance was consistent between the five cycles performed, with an average yield of 88%±1% standard deviation. The five chromatograms from the MabSelect™ SuRe™ chromatography cycles share a very similar elution profile. The product yield was approximately 100% following low pH inactivation and neutralization (Table 21). However, the turbidity increased approximately 9.8-fold to an average of 105 NTU (Table 21) and there was 37.53% soluble protein aggregates present in the neutralized low pH inactivation pool in the Schematic 1 process (Table 22).

TABLE 20
Protein A Capture Chromatography Data

| | | | | | | | | Tur- |
|---|---|---|---|---|---|---|---|---|
| | Load | Load | Resin Load | Pool | Pool | Pool | Yield | bidity |
| Cycle | (mL) | (mg/mL) | (mg/mL) | (mL) | (mg/mL) | (CV) | (%) | (NTU) |
| 1 | 3800 | 0.6 | 21.5 | 509 | 4.03 | 4.8 | 90% | 12.7 |
| 2 | 3800 | 0.6 | 21.5 | 549 | 3.67 | 5.2 | 88% | 10.6 |
| 3 | 3800 | 0.6 | 21.5 | 576 | 3.47 | 5.4 | 88% | 10.2 |
| 4 | 3800 | 0.6 | 21.5 | 593 | 3.33 | 5.6 | 87% | 9.2 |
| 5 | 3800 | 0.6 | 21.5 | 570 | 3.50 | 5.4 | 88% | 10.4 |

| | |
|---|---|
| Average ± SD | 88 ± 1% |

1 CV = 106 mL

TABLE 21
Low pH Viral Inactivation Data

| | Load | Load | Pool | Pool | | Tur- |
|---|---|---|---|---|---|---|
| | Vol | onc | Vol | Conc | Yield | bidity |
| Cycle | (mL) | (mg/mL) | (mL) | (mg/mL) | (%) | (NTU) |
| 1 | 499 | 4.03 | 520 | 3.69 | 95 | 116 |
| 2 | 539 | 3.67 | 558 | 3.38 | 95 | 105 |
| 3 | 566 | 3.47 | 588 | 3.47 | 104 | 113 |
| 4 | 583 | 3.33 | 602 | 3.33 | 103 | 92.4 |
| 5 | 560 | 3.50 | 579 | 3.5 | 103 | 98.4 |

TABLE 22
Eculizumab Size Exclusion Chromatography-HPLC Results

| Sample Description | % Aggregate | % Main Peak | % Fragment |
|---|---|---|---|
| Neut. Low pH Pool | 37.53 | 61.66 | 0.81 |
| (Cycle 1) | | | |
| Delipid Filtrate | 2.89 | 96.58 | 0.52 |
| (Pooled Cycles) | | | |
| CA ImpRes Pool | 0.60 | 99.39 | 0 |
| (Schematic 1) | | | |
| Viral Filtrate | 0.61 | 99.40 | 0 |
| (Schematic 1) | | | |
| POROS 50HS Pool | 0.39 | 99.17 | 0.44 |
| (Schematic 2) | | | |
| CA ImpRes Pool | 0.26 | 99.74 | 0 |
| (Schematic 2) | | | |
| UF/DF1 Retentate | 3.86 | 95.54 | 0.59 |
| (Schematic 3) | | | |
| POROS 50HQ Pool | 3.56 | 95.89 | 0.55 |
| (Schematic 3) | | | |
| CA ImpRes Pool | 0.64 | 99.36 | 0 |
| (Schematic 3) | | | |

Delipid filtration was performed with three 25-cm² filters in parallel (total area of 0.0075 m²). The Delipid depth filtration results are shown in Table 23. The amount of eculizumab loaded on the Delipid filter ranged from 249 g/m² to 270 g/m² and the process yields ranged from 42% to 52%, with an average of 46%±4%. The Delipid pools from the five cycles were pooled and then analyzed by size exclusion chromatography-HPLC (SEC-HPLC). The Delipid depth filtration step reduced the level of soluble protein aggregates from 37.53% to 2.89% (FIG. 6 and Table 22).

TABLE 23
Delipid Depth Filtration Data

| | Load | Load | Filter | Pool | Pool | Filter | |
|---|---|---|---|---|---|---|---|
| | Vol | Conc | Load | Vol | Conc | Load | Yield |
| Cycle | (mL) | (mg/mL) | (L/m²) | (mL) | (mg/mL) | (g/m²) | (%) |
| 1 | 515 | 3.69 | 69 | 880 | 1.13 | 253 | 52% |
| 2 | 553 | 3.38 | 74 | 929 | 0.97 | 249 | 48% |
| 3 | 583 | 3.47 | 78 | 951 | 0.90 | 270 | 42% |
| 4 | 597 | 3.33 | 80 | 971 | 0.85 | 265 | 42% |
| 5 | 574 | 3.5 | 77 | 939 | 1.00 | 268 | 47% |

| | |
|---|---|
| Average ± SD | 46 ±4% |

Total filter area = 0.0075 (m²)

The data from the POROS 50HS chromatography step is shown in Table 24. The pool volume was 449 mL, which was 11 mL less than the load volume, and the yield was 92%. As a result of the POROS 50HS column at the first polishing step, the Schematic 2 process removed the most amount of aggregate to a final level of 0.26% following the Capto Adhere ImpRes step (FIG. 6 and Table 22).

TABLE 24
POROS 50HS Chromatography Data

| | Load | Load | Resin | Pool | Pool | Pool | |
|---|---|---|---|---|---|---|---|
| | Vol | Conc | Load | Vol | Conc | Volume | Yield |
| Cycle | (mL) | (mg/mL) | (mg/mL) | (mL) | (mg/mL) | (CV) | (%) |
| 1 | 500 | 0.92 | 20.3 | 449 | 0.94 | 19.86 | 92 |

The UF/DF1 data are shown in Table 25. UF/DF1 filter area was 0.005 m² and the average yield was 96%±2%. UF/DF1 caused an increase in the level of soluble protein aggregates from 2.89% to 3.86%.

TABLE 25
UF/DF1 Data

| | Load | Load | Filter | Pool | Pool | |
|---|---|---|---|---|---|---|
| | Vol | Conc | Load | Vol | Conc | Yield |
| Cycle | (mL) | (mg/mL) | (g/m2) | (mL) | (mg/mL) | (%) |
| 1 | 512 | 0.98 | 100.35 | 142 | 3.33 | 94 |
| 2 | 510 | 0.98 | 99.96 | 128 | 3.8 | 97 |

| | |
|---|---|
| Average ± SD | 96 ± 2% |

The POROS 50HQ chromatography data are shown in Table 26. The pool volume was ˜8% greater than the load volume. The yield was 99% and the level of soluble protein aggregates was slightly reduced from 3.86% in the material loaded to 3.56% in the POROS 50HQ pool (Table 22).

TABLE 26
POROS 50HQ Chromatography Data

| | Load | Load | Resin | Pool | Pool | |
|---|---|---|---|---|---|---|
| | Vol | Conc | Load | Vol | Conc | Yield |
| Cycle | (mL) | (mg/mL) | (mg/mL) | (mL) | (mg/mL) | (%) |
| 1 | 122 | 3.8 | 0.91 | 132 | 3.49 | 99 |

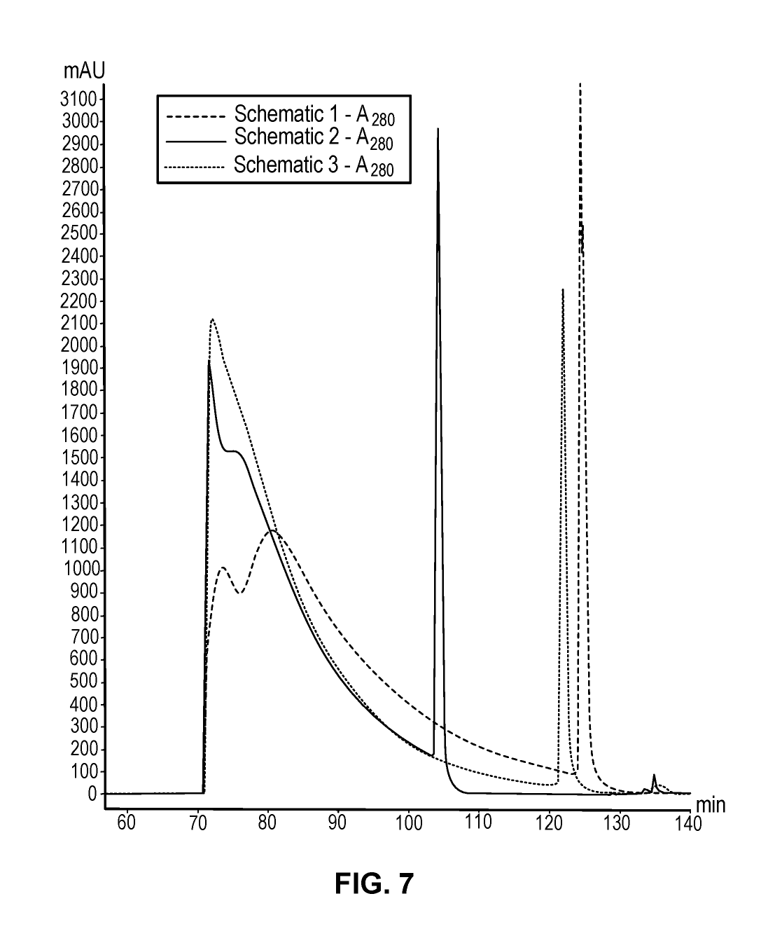

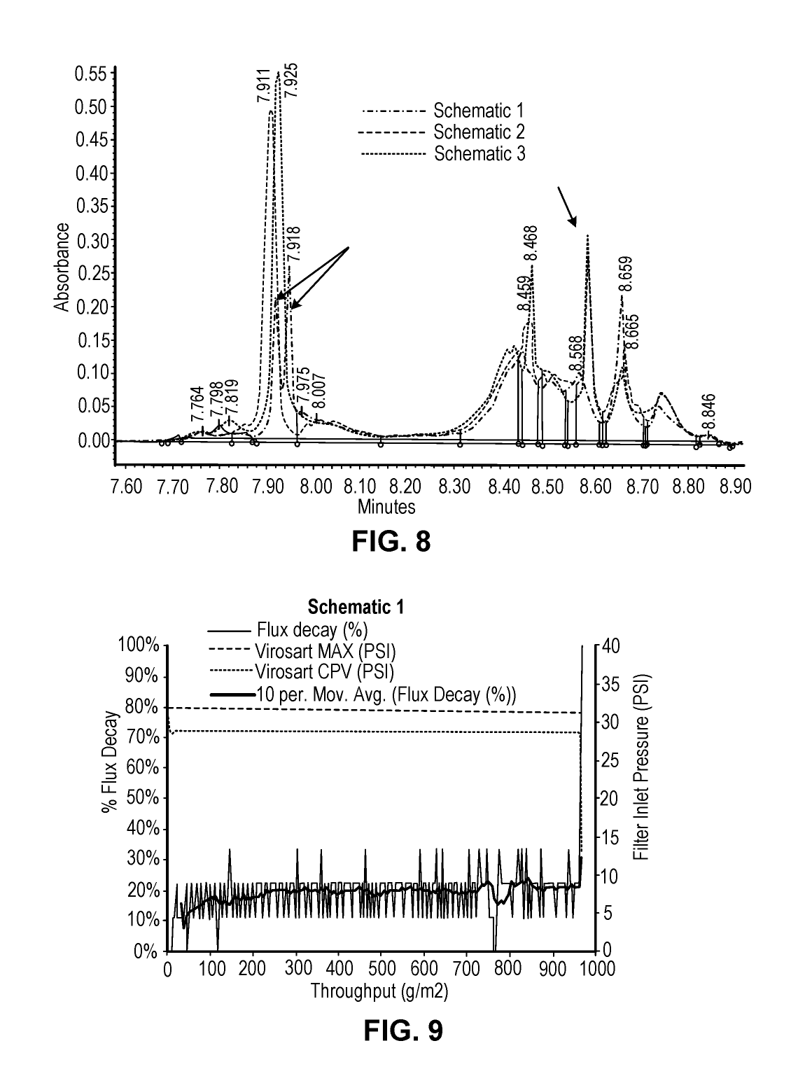

The data from the Capto Adhere ImpRes chromatography step performed in each of the processes of Schematics 1 to 3 are shown in Table 27. The Capto Adhere ImpRes chromatography in Schematic 1 was performed using a 2.5 cm×2.1 cm column with a column volume of 103 mL, while the Capto Adhere ImpRes chromatography in Schematics 2 and 3 were run using a 1 cm×19.4 cm column with a column volume of 15.2 mL. The concentration of the material loaded varied between Schematics 1 to 3, however the resin loads were held constant and averaged 19.2 mg/mL. The yield from Schematics 1 to 3 was an average of 81%±4%. FIG. 7 is an overlay of the Capto Adhere ImpRes chromatograms from each of Schematics 1 to 3. The chromatograms show differences in the peak shapes, with Schematics 2 and 3 showing similar but distinct trends. These differences likely reflect different properties (i.e., charge) of adalimumab following the different upstream purification/filtration steps performed in each of Schematics 1 to 3. The isoelectric focusing capillary electrophoresis data shows that distinct charge variants (peaks) were observed between Schematics 1, 2, and 3 Capto Adhere ImpRes pools (FIG. 8). More acidic species were present in the Capto Adhere ImpRes pools as compared to all of the other isoelectric focusing capillary electrophoresis fractions with the POROS 50HS pool being the only exception. The aggregate levels achieved at the end of each of Schematics 1 to 3 was 0.6% (Table 22).

TABLE 27
Capto Adhere ImpRes Chromatography Data

| | Load | Load | Resin | Pool | Pool | |
|---|---|---|---|---|---|---|
| | Vol | Conc | Load | Vol | Conc | Yield |
| Cycle | (mL) | (mg/mL) | (mg/mL) | (mL) | (mg/mL) | (%) |
| Schematic 1 * | 2051 | 0.96 | 19.1 | 628 | 2.4 | 77 |
| Schematic 2 ** | 300 | 0.94 | 18.6 | 76 | 3.0 | 81 |
| Schematic 3 ** | 89 | 3.42 | 20.0 | 78 | 3.27 | 84 |
| | | | | | Average ± SD | 81 ± 4% |

* CV = 103 mL
** CV = 15.2 mL

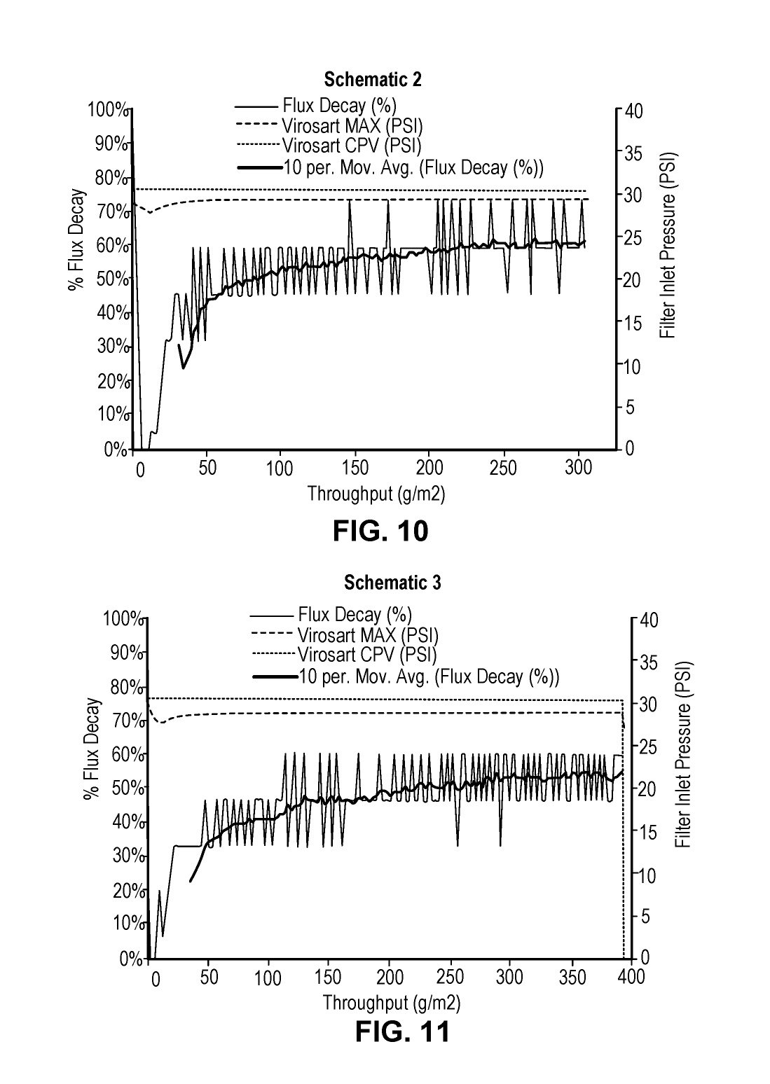

The viral filtration data is shown in Table 28. The average yield was 97%±1%. Eculizumab flux stabilized at approximately 300 g/m² (FIGS. 9-11) and remained stable up to 950 g/m² (FIG. 9). FIGS. 9-11 show the flux decay and filter inlet pressure versus volumetric throughput. Viral filtration of material in Schematic 1 did not cause an increase in the percentage of soluble protein aggregates as determined by size exclusion chromatography-HPLC (SEC-HPLC) (Table 22).

TABLE 28
Viral Filtration Data

| | Load | Load | Filter | Filter | Pool | Pool | |
|---|---|---|---|---|---|---|---|
| | Vol | Conc | Load | Load | Vol | Conc | Yield |
| Cycle | (mL) | (mg/mL) | (L/m²) | (g/m²) | (mL) | (mg/mL) | (%) |
| Schematic 1 | 205 | 2.4 | 410 | 984 | 208 | 2.3 | 96 |
| Schematic 2 | 47 | 3 | 94 | 282 | 57 | 2.4 | 97 |
| Schematic 3 | 60 | 3.27 | 120 | 392.4 | 66 | 2.9 | 98 |

| | |
|---|---|
| Average ± SD | 97 ± 1% |

SEC-HPLC analyses was performed on eculizumab process intermediates following a single freeze-thaw cycle was conducted to determine its effect on soluble protein aggregate levels (Table 29).

TABLE 29
Freeze/Thaw SEC-HPLC Data

| Sample Description | % Aggregate | % Main Peak | % Fragment |
|---|---|---|---|
| Freeze Thaw/Capto Adhere | 2.86 | 96.58 | 0.56 |
| ImpRes Load (Schematic 1) | | | |
| Freeze Thaw/Capto Adhere | 0.73 | 99.27 | 0 |
| ImpRes Pool (Schematic 1) | | | |
| Freeze Thaw/POROS | 2.90 | 96.60 | 0.50 |
| 50HS Load (Schematic 2) | | | |
| Freeze Thaw/POROS | 0.51 | 99.02 | 0.47 |
| 50HS Pool (Schematic 2) | | | |
| Freeze Thaw/POROS | 3.70 | 95.69 | 0.61 |
| 50HQ Load (Schematic 3) | | | |

All the tested processes (Schematics 1 to 3) effectively removed aggregates to 0.6% soluble protein aggregates and all resulted in similar product yields (from 25% to 27%) through viral filtration. The Schematic 2 process, which included the POROS 50HS chromatography for the first polishing step, removed the most soluble protein aggregates, to a level of 0.3%. In Schematic 3, the UF/DF1 step prior to the POROS 50HQ chromatography step caused an increase in the level of soluble protein aggregates (from 2.9% to 3.9%). Taken together, these data demonstrate that all three tested processes (Schematics 1 to 3) produced similar product yields and effectively cleared soluble protein aggregates.

### Example 5

### Optimization of Protein A Chromatography Elution and Depth Filtration

A set of experiments were performed to assess the minimum amount of sodium chloride required in the protein A elution buffer to prevent precipitation and aggregation

### Materials and Methods

A clarified culture medium including eculizumab obtained from a single bioreactor culture was used in these experiments. MabSelect™ SuRe™ from GE Healthcare media (Catalogue No. 17-5438-05, Lot No. 10037980) was used to prepare a 2.5 cm×20 cm column having a column volume of 106 mL. A Millipore Pellicon 3 UF/DF membrane was used to perform ultrafiltration/diafiltration. An Orion Conductivity probe (E12/026) was used to measure conductivity, and a conductivity meter was used following standardization with 100 mS/cm conductivity buffer at the start of every day. A Hach Turbidity meter (Catalog No. 470060, Serial No. 05080C020561) was used to measure turbidity of a fluid.

A summary of the protein A chromatography and the ultrafiltration/diafiltration methods used in this Example is shown in Table 30. MabSelect™ SuRe™ chromatography was performed as outlined in Tables 31 and 32. Eculizumab was eluted from the column using a buffer of 25 mM sodium acetate, 150 mM sodium chloride, pH 3.7.

TABLE 30
Summary of Protein A Chromatography and
Ultrafiltration/Diafiltration Methods

| Protein A | Protein A | UF/DF1 | UF/DF1 |
|---|---|---|---|
| Column Size | Column Cycles | (cm²) | Load |
| 2.6 cm D × 20 cm | 1 | 88, 30 kDa | 100 g/m² |
| H/106 mL | (2.5 g per cycle) | MWCO | (~0.9 g) |

TABLE 31
Protein A Chromatography Conditions

| Resin type: | MabSelect ™ SuRe ™ |
|---|---|
| Column diameter (cm): | 2.6 |
| Column height estimated (cm): | 20 |
| Column volume (mL): | 106 |
| Load (mg/mL resin): | ~25 |
| Amount loaded x cycle (mg): | ~2450 |

TABLE 32
Protein A Chromatography Conditions

| | | | Volume/Collection | | Flow Direction/ |
|---|---|---|---|---|---|
| Step | Block Name | Solution | Criteria | | Flow rate cm/hr |
| 1. | Equilibration | 50 mM Sodium Phosphate, | ≧4.0 | CV | Downflow/300 |
| | | 100 mM Sodium Chloride, | | | |
| | | pH 7.00 | | | |
| 2. | Load | Clarified Harvest | 32 mg/mL resin | | Downflow/300 |
| 3. | Post-load | 50 mM Sodium Phosphate, | ≧4.0 | CV | Downflow/300 |
| | wash 1 | 100 mM Sodium Chloride, | | | |
| | | pH 7.00 | | | |
| 4. | Post-load | 85 mM Sodium Phosphate, | ≧5.0 | CV | Downflow/300 |
| | wash 2 | 100 mM Sodium Chloride, | | | |
| | | 0.7% Caprylic acid, | | | |
| | | 300 mM Arginine, pH 7.5 | | | |
| 5. | Post-load | 50 mM Sodium Phosphate, | ≧4.0 | CV | Downflow/300 |
| | wash 3 | 100 mM Sodium Chloride, | | | |
| | | pH 7.00 | | | |
| 6. | Elution | *See Table 9 below | 100 mAU-100 mAU | | Downflow/300 |
| | | | (2 mm path-length) | | |
| 7. | Post-elution | 0.1M Citric acid | ≧3.0 | CV | Downflow/300 |
| | Strip | | | | |
| 8. | Post Strip | dH₂O | ≧3.0 | CV | Downflow/300 |
| | Flush | | | | |
| 9. | Clean | 0.1N NaOH | 3.0 | CV | Upflow/300 |
| 10. | Static Hold | 0.1N NaOH | 60 | min | N/A |
| 11. | Pre-Store | 50 mM Sodium Phosphate, | ≧3.0 | CV | 1 CV |
| | Flush | 100 mM Sodium Chloride, | | | Upflow/2 CV |
| | | pH 7.00 | | | Downflow/300 |
| 12. | Storage | 18% Ethanol | ≧3.0 | CV | Downflow/300 |

A buffer exchange step was performed on the protein A elution to assess the minimum amount of sodium chloride required in the protein A elution buffer to prevent precipitation and aggregation. The diafiltration buffer used was 25 mM sodium acetate, pH 3.7. A buffer exchange of six diavolumes was performed and conditions are shown in Table 33.

TABLE 33
UF/DF1 Process Conditions
Diafiltration

| TFF membrane used | Millipore Pellicon 3 | |
|---|---|---|
| Membrane cutoff (Da) | 30,000 | |
| TMP | 1.0-1.5 | bar |
| Feed flow rate | 4-8 | L/m²/min |

| | |
|---|---|
| Filter area (m²) | 0.0088 |
| Filter load (g/m²) | ≦100 |
| Diavolumes | 6X |
| Diaflltration Buffer | 25 mM Sodium Acetate, pH 3.7 |

### Results

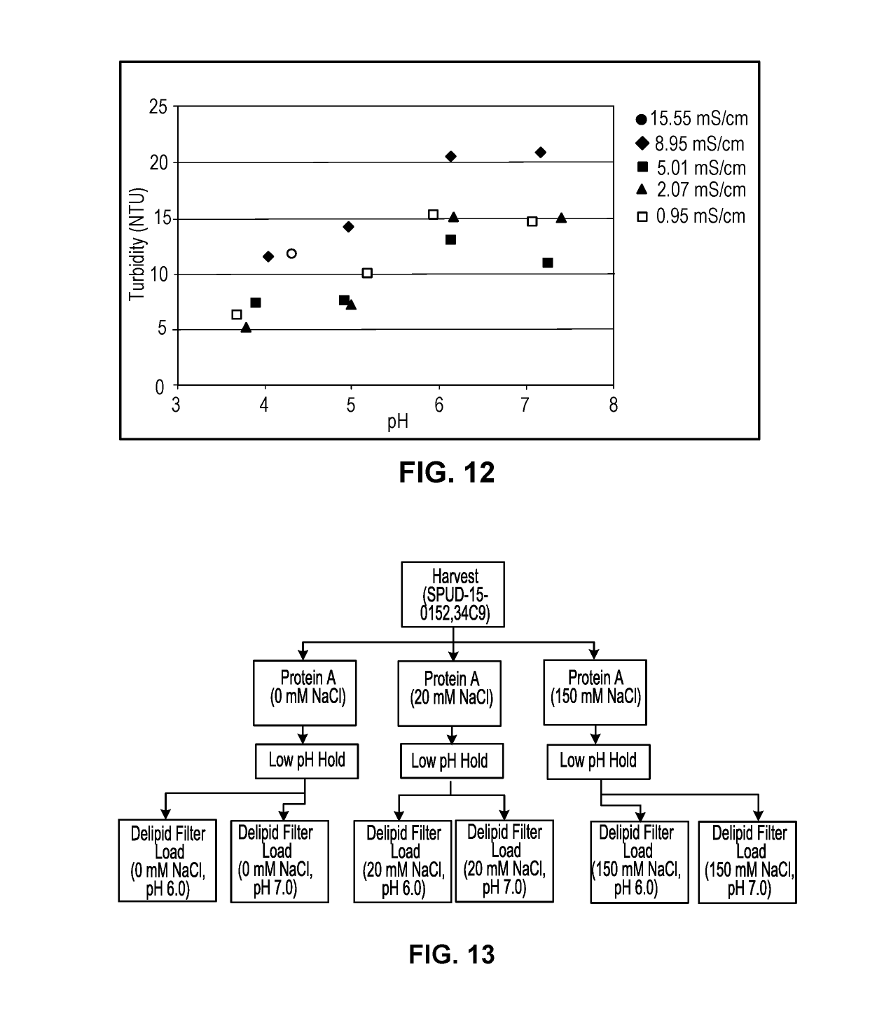

Tables 34 and 35, and FIG. 12 show the results of these experiments. The data show that a protein A pool with 150 mM NaCl resulted in an 80% yield (Table 34). The data also show that as the conductivity and pH decreased in the Protein A pool by diafiltration, the turbidity also decreased (Table 35 and FIG. 12). The data also show that eculizumab will not precipitate at a concentration of approximately 4 mg/mL in 25 mM acetate buffer between pH 4 and 7 with a conductivity of <1-15 mS/cm (<10-150 mM NaCl) at room temperature and 2° C. to 8° C.

TABLE 34
Protein A Chromatography Results

| Load | Load | Resin | Pool | Pool | Pool | |
|---|---|---|---|---|---|---|
| Vol | Conc | Load | Vol | Conc | Vol | Yield |
| (L) | (g/L) | (g/L) | (L) | (g/L) | (CV) | (%) |
| 2.57 | 1.03 | 25 | 0.544 | 3.86 | 5.1 | 80 |

TABLE 35
Protein A Pool Turbidity versus pH and Conductivity Results

| Conductivity | pH | Turbidity |
|---|---|---|
| 15.55 mS/cm | 4.31 | 11.7 |
| 8.95 mS/cm | 4.05 | 11.7 |
| | 4.95 | 14.2 |
| | 6.14 | 20.5 |
| | 7.16 | 20.8 |
| 5.01 mS/cm | 3.9 | 7.2 |
| | 4.92 | 7.5 |
| | 6.14 | 13 |
| | 7.27 | 10.8 |
| 2.07 mS/cm | 3.76 | 5.2 |
| | 4.99 | 7.2 |
| | 6.15 | 15.1 |
| | 7.39 | 15 |
| 0.95 mS/cm | 3.69 | 6.2 |
| | 5.18 | 9.9 |
| | 5.93 | 15.2 |
| | 7.08 | 14.6 |

### Example 6

### Optimization of Depth Filtration Step

A set of experiments was performed to test three different protein A chromatography elution buffers. Each protein A chromatography elution pool was treated with low pH to achieve viral inactivation and was neutralized to the desired pH (pH of 6 or 7), prior to passing it through a Zeta Plus Delipid depth filter. A total of six different load conditions were filtered through the Delipid depth filter (25 cm² filtration area) at a load of 100 L/m² and flow of 150 LMH.

### Materials and Methods

A clarified culture medium including eculizumab obtained from a single bioreactor culture was used in these experiments. MabSelect™ SuRe™ from GE Healthcare media (Catalogue No. 17-5438-05, Lot No. 10037980) was used to prepare a 2.5 cm×20 cm column having a column volume of 106 mL. A 3M Zeta Plus Delipid depth filter (Catalog No. BC0025LDELI08A, Lot Nos. 43720 and 43534) were used to perform the step of depth filtration. An Explorer and Avant automated liquid chromatography system was used to perform the Protein A column chromatography. An Orion Ross ultra-flat surface pH probe (Sx1-15902) and a pH meter standardized at the start of every day with pH 4, 7, and 10 buffer was used to detect pH.

A summary of the methods used in the six different tested processes in this Example are shown in FIG. 13. A summary of the protein A chromatography and the depth filtration methods used in this Example is shown in Table 36. MabSelect™ SuRe™ chromatography was performed as outlined in Tables 31 and 32 (shown in Example 5). In contrast to the Protein A chromatography methods described in Example 5, eculizumab in the experiments in this Example was eluted from the protein A column using one of the three different elution buffers shown in Table 37.

TABLE 36
Summary of Protein A Chromatography
and Depth Filtration Methods

| Protein A | Protein A | Delipid | Delipid |
|---|---|---|---|
| Column Size | Column Cycles | Filter Size | Filter Load |
| 2.6 cm D × | 3 | 25 cm² × 6 | 100 L/m² |
| 20 cm H/106 mL | (2.5 g per cycle) | (DELI08A) | (250 mL × 6) |

TABLE 37
Protein A Chromatography Elution Conditions

| Cycle # | Block Name | Solution |
|---|---|---|
| 1 | Elution | 25 mM Sodium Acetate 150 mM NaCl, pH 3.7 |
| 2 | Elution | 25 mM Sodium Acetate, pH 3.7 |
| 3 | Elution | 25 mM Sodium Acetate 20 mM NaCl, pH 3.7 |

The low pH step was performed to inactivate viruses. In each of the six tested processes in this Example, low pH viral inactivation was performed using the protocol summarized in Table 38. Neutralization was performed using 1.0 M Tris base.

TABLE 38
Low pH Viral Inactivation Process Conditions

| Sub-Step | Parameter | Target/Range |
|---|---|---|
| Acidification | Final Target pH | 3.70 ± 0.10 |
| | Addition rate | 2 mL/min/L of pool |
| | Hold Time | 60-70 min |
| Neutralization | Final Target pH | 6.00 ± 0.10 |
| | Addition rate | 4 mL/min/L of pool |

Following the low pH viral inactivation protocol, the solutions including eculizumab were filtered through a Zeta Plus Delipid depth filter (25 cm² filtration area). The solutions including eculizumab were loaded onto the Delipid depth filter at ≦100 L/m². Filtration was performed at 150 LMH. A 50 L/m² recovery flush was executed using flush buffer (50 mM sodium phosphate, 150 mM NaCl, pH 6.00). The Delipid depth filtration was performed using the conditions shown in Table 39 below.

TABLE 39
Depth Filtration Protocol

| Process Parameter | Target/Range |
|---|---|
| Filter Type | 3M DELI08A |
| Flush Buffer | 50 mM Sodium phosphate, |
| | 150 mM NaCl, pH 6 |
| Flush volume (L/m²) | 54 |
| Membrane Surface Area (cm²) | 25 |
| Volume to Load (mL) | 1 Protein A Cycle |
| Load volume (L/m²) | ≦100 |
| Post Load Flush (L/m²) | 50 |
| Flow Rate (LMH) | 150 |

### Results

Tables 40-42 show the protein A chromatography data and the low pH viral inactivation data. As the NaCl concentration increased in the protein A chromatography elution buffers, the pool volume decreased and the yield increased. However, the turbidity increased as the NaCl concentration in the protein A elution buffer increased (Table 40). The protein A pools neutralized to pH 6 or 7 (after the low pH hold) increased in the level of turbidity (Table 41) and the level of soluble protein aggregates (Table 42) as the NaCl concentration increased. The turbidity substantially increased from the protein A pool to the neutralized protein A pool (following low pH treatment and neutralization) when comparing the same NaCl concentration (Tables 40 and 41).

TABLE 40
Protein A Chromatography Data

| | NaCl | Load | Load | Resin | Pool | Pool | Pool | | Tur- |
|---|---|---|---|---|---|---|---|---|---|
| Cycle | Concentration | Vol | Conc | Load | Vol | Conc | Vol | Yield | bidity |
| # | (mM) | (L) | (g/L) | (g/L) | (L) | (g/L) | (CV) | (%) | (NTU) |
| 2 | 0 | 2.57 | 1.03 | 25 | 0.689 | 2.73 | 6.5 | 71 | 5.11 |
| 3 | 20 | 2.57 | 1.03 | 25 | 0.509 | 4.33 | 4.8 | 83 | 9.51 |
| 1 | 150 | 2.57 | 1.03 | 25 | 0.354 | 6.59 | 3.3 | 88 | 28.2 |

TABLE 41
Low pH Viral Inactivation Data

| | NaCl | | Load | Load | Pool | Pool | | Tur- |
|---|---|---|---|---|---|---|---|---|
| Cycle | Conc. | | Volume | Conc. | Volume | Conc. | *Yield | bidity |
| # | (mM) | pH | (L) | (g/L) | (L) | (g/L) | (%) | (NTU) |
| 2 | 0 | 6 | | 2.73 | | 2.67 | | 38.1 |
| | | 7 | | 2.73 | | 2.56 | | 28.6 |
| 3 | 20 | 6 | | 4.33 | | 4.16 | | 26.7 |
| | | 7 | | 4.33 | | 4.18 | | 27.6 |
| 1 | 150 | 6 | 0.1853 | 6.59 | 0.1853 | 6.24 | 95 | 49.5 |
| | | 7 | 0.1872 | 6.59 | 0.1872 | 6.20 | 94 | 72.3 |

TABLE 42
Low pH Viral Inactivation Impurities

SEC

| Description | % Aggregate | % Monomer | % Fragment |
|---|---|---|---|
| Neut. Pro A Pool | 28.10 | 70.90 | 0.99 |
| (0 mM NaCl) | | | |
| Neut. Pro A Pool | 39.91 | 59.17 | 0.92 |
| (20 mM NaCl) | | | |
| Neut. Pro A Pool | 45.78 | 53.29 | 0.94 |
| (150 mM NaCl) | | | |

Tables 43-45 show the Delipid load and filtration data. The data show that increasing the NaCl of the depth filter loading material increases the level of soluble protein aggregate and host cell protein clearance (Table 43). The Delipid depth filtration process (loaded at 72 L/m²) for pH 6 and 7 resulted in a slightly higher yield, but also higher turbidity, as the NaCl concentration increased (Table 44). The results for the Delipid depth filter filtrate at pH 6 and 7 show the same trend as the Delipid depth filter load for the level of soluble protein aggregates and host cell protein clearance. Delipid loading material having a pH of 6 had the lowest level of impurities as compared to Delipid loading material having a pH of 7 with the same amount of NaCl, and resulted in approximately 1 log reduction in host cell protein levels (Table 45) as compared to a starting Delipid loading material at pH 7 with 0 and 20 mM NaCl (Table 43). When a Delipid loading material including 150 mM NaCl was used, the result was a 0.2 log reduction in host cell protein levels in the filtrate. The data show that a Delipid depth filter loading material having a pH of 6 with no added NaCl had the highest soluble protein aggregate and host cell protein removal/clearance when passed through a Delipid depth filter.

TABLE 43
Delipid Depth Filtration Impurities

| NaCl | | | | | |
|---|---|---|---|---|---|
| Concentration | | SEC | | | HCP |
| (mM) | pH | % Aggregate | % Monomer | % Fragment | (ng/mg) |
| 0 | 6 | 32.01 | 67.12 | 0.86 | |
| 0 | 7 | 31.25 | 67.85 | 0.90 | 412 |
| 20 | 6 | 42.26 | 56.89 | 0.85 | |
| 20 | 7 | 42.28 | 56.82 | 0.91 | 345 |
| 150 | 6 | 48.77 | 50.54 | 0.99 | |
| 150 | 7 | 48.99 | 49.98 | 1.03 | 915 |

TABLE 44
Delipid Depth Filtration Data

| | NaCl | | Load | Load | Pool | Pool | | Tur- |
|---|---|---|---|---|---|---|---|---|
| Cycle | Conc. | | Volume | Conc. | Volume | Conc. | Yield | bidity |
| # | (mM) | pH | (L) | (g/L) | (L) | (g/L) | (%) | (NTU) |
| 2 | 0 | 6 | 0.180 | 2.67 | 0.303 | 0.82 | 52 | 1.28 |
| | | 7 | 0.180 | 2.56 | 0.302 | 0.74 | 49 | 1.21 |
| 3 | 20 | 6 | 0.180 | 4.16 | 0.305 | 1.46 | 59 | 1.71 |
| | | 7 | 0.180 | 4.18 | 0.299 | 1.39 | 55 | 1.81 |
| 1 | 150 | 6 | 0.180 | 6.24 | 0.304 | 2.19 | 59 | 2.47 |
| | | 7 | 0.183 | 6.20 | 0.303 | 2.04 | 55 | 2.99 |

TABLE 45
Delipid Depth Filtration Impurities

| NaCl | | | | | |
|---|---|---|---|---|---|
| Concentration | | SEC | | | HCP |
| (mM) | pH | % Aggregate | % Monomer | % Fragment | (ng/mg) |
| 0 | 6 | 6.51 | 92.53 | 0.96 | 22 |
| 0 | 7 | 8.83 | 90.02 | 1.14 | 75 |
| 20 | 6 | 19.96 | 79.08 | 0.96 | 30 |
| 20 | 7 | 20.42 | 78.51 | 1.07 | 86 |
| 150 | 6 | 30.52 | 68.76 | 0.72 | 114 |
| 150 | 7 | 30.10 | 68.86 | 1.04 | 548 |

### Example 7

### Optimization of Depth Filtration to Maximize Antibody Recovery and Impurity Clearance

An additional set of experiments were performed to test different Delipid depth filter loading conditions in an effort to optimize antibody recovery and removal of impurities (host cell protein and soluble protein aggregates).

### Materials and Methods

A clarified culture medium including a biparatopic antibody of Alexion 1210 obtained from a single bioreactor culture was used in these experiments. MabSelect™ SuRe′ from GE Healthcare media (Catalogue No. 17-5438-05, Lot No. 10037980) was used to prepare a 2.5 cm×20 cm column having a column volume of 106 mL. A 3M Zeta Plus Delipid depth filter (Catalog No. BC0025LDELI08A, Lot Nos. 43720 and 43534) were used to perform the step of depth filtration. An Explorer and Avant automated liquid chromatography system was used to perform the Protein A column chromatography. An Orion Ross ultra-flat surface pH probe (Sx1-15902) and a pH meter standardized at the start of every day with pH 4, 7, and 10 buffer was used to detect pH.

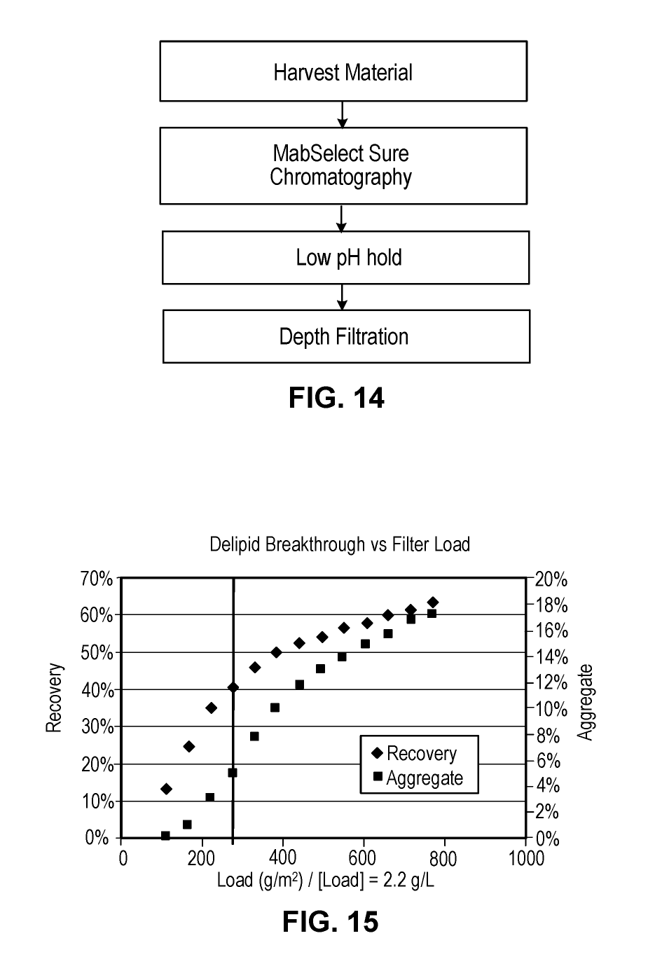

A summary of the methods used in the six different tested processes in this Example are shown in FIG. 14. A summary of the protein A chromatography and the depth filtration methods used in this Example is shown in Table 46. MabSelect™ SuRe™ chromatography was performed as outlined in Tables 31 and 32 (shown in Example 5). In contrast to the Protein A chromatography methods described in Example 5, the antibody in the experiments in this Example was eluted from the protein A column using 25 mM sodium acetate, pH 3.7.

TABLE 46
Summary of Protein A Chromatography and Depth Filtration

| Protein A | Protein A | Delipid | Delipid |
|---|---|---|---|
| Column Size | Column Cycles | Filter Size | Filter Load |
| 2.6 cm D × | 1 | 25 cm² × 1 | 1 Protein A Cycle |
| 20 cm H/106 mL | (2.5 g per cycle) | (DELI08A) | at 150 LMH |

The low pH step was performed to inactivate viruses. The low pH viral inactivation was performed using the protocol summarized in Table 38 (shown in Example 6). Neutralization was performed using 1.0 M Tris base.

Following the low pH viral inactivation protocol, the solutions including the antibody were filtered through a Zeta Plus Delipid depth filter (25 cm2 filtration area). The solutions including the antibody were loaded onto the Delipid depth filter at >200 L/m². Filtration was performed at 150 LMH. A 50 L/m² recovery flush was executed using flush buffer (50 mM sodium phosphate, pH 6). The Delipid depth filtration was performed using the conditions shown in Table 47 below.

TABLE 47
Delipid Depth Filter Conditions

| Process Parameter | Target/Range |
|---|---|
| Filter Type | 3M DELI08A |
| Flush Buffer | 50 mM Sodium phosphate, pH 6 |
| Flush volume (L/m²) | 54 |
| Membrane Surface Area (cm²) | 25 |
| Volume to Load (mL) | 1 Protein A Cycle |
| Load volume (L/m²) | >200 |
| Post Load Flush (L/m²) | 50 |
| Flow Rate (LMH) | 150 |

### Results

Tables 48 and 49 show the protein A and low pH viral inactivation data. Tables 50-52 and FIG. 15 show the Delipid depth filtration data. These data indicate that the Zeta Plus Delipid filter may be loaded at ≦275 g/m² to optimize the purification of the antibody.

TABLE 48
Protein A Chromatography Data

| Load | Load | Resin | Pool | Pool | Pool | | Tur- |
|---|---|---|---|---|---|---|---|
| Vol | Conc | Load | Vol | Conc | Vol | Yield | bidity |
| (L) | (g/L) | (g/L) | (L) | (g/L) | (CV) | (%) | (NTU) |
| 2.57 | 1.03 | 25.0 | 0.881 | 2.22 | 8.3 | 74 | 4.5 |

TABLE 49
Low pH Viral Inactivation Data

| | Load | Load | Pool | Pool | | Tur- |
|---|---|---|---|---|---|---|
| Cycle | Volume | Conc. | Volume | Conc. | Yield | bidity |
| # | (L) | (g/L) | (L) | (g/L) | (%) | (NTU) |
| 1 | 0.874 | 2.22 | 0.898 | 2.20 | 102 | 29.0 |

TABLE 50
Delipid Depth Filtration Impurity Data

| SEC | | | HCP |
|---|---|---|---|
| % Aggregate | % Monomer | % Fragment | (ng/mg) |
| 33.65 | 65.93 | 0.43 | 162 |

TABLE 51
Delipid Depth Filtration Recovery
and Breakthrough Process Results

| | | Load Conc. | Volume | Conc. | % |
|---|---|---|---|---|---|
| L/m² | g/m² | (mg/mL) | (mL) | (mg/mL) | Recovery |
| 50 | 110 | 2.20 | 125 | 0.29 | 13.2 |
| 75 | 165 | 2.20 | 188 | 0.55 | 25.0 |
| 100 | 220 | 2.20 | 250 | 0.78 | 35.5 |
| 125 | 275 | 2.20 | 313 | 0.91 | 41.4 |
| 150 | 330 | 2.20 | 375 | 1.02 | 46.4 |
| 175 | 385 | 2.20 | 438 | 1.10 | 50.0 |
| 200 | 440 | 2.20 | 500 | 1.16 | 52.7 |
| 225 | 495 | 2.20 | 563 | 1.20 | 54.5 |
| 250 | 550 | 2.20 | 625 | 1.25 | 56.8 |
| 275 | 605 | 2.20 | 688 | 1.28 | 58.2 |
| 300 | 660 | 2.20 | 750 | 1.33 | 60.5 |
| 325 | 715 | 2.20 | 813 | 1.35 | 61.4 |
| 350 | 770 | 2.20 | 875 | 1.40 | 63.6 |

TABLE 52
Delipid Depth Filtration Recovery
and Breakthrough Impurity Results

| SEC | | | HCP |
|---|---|---|---|
| % Aggregate | % Monomer | % Fragment | (ng/mg) |
| 0.18% | 99.33% | 0.49% | 14 |
| 1.03% | 98.57% | 0.40% | |
| 3.16% | 96.47% | 0.37% | |
| 4.97% | 94.66% | 0.37% | 13 |
| 7.73% | 91.89% | 0.38% | |
| 10.01% | 89.67% | 0.32% | |
| 11.75% | 87.88% | 0.36% | |
| 13.00% | 86.63% | 0.37% | |
| 13.85% | 85.76% | 0.39% | 13 |
| 14.91% | 84.72% | 0.37% | |
| 15.73% | 83.85% | 0.43% | |
| 16.69% | 82.94% | 0.37% | |
| 17.28% | 82.35% | 0.37% | 18 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of purifying a recombinant protein, the method comprising:
(a) capturing a recombinant protein from a solution comprising the recombinant protein;
(b) following capturing, performing one or more unit operations on the solution; and
(c) following steps (a) and (b), flowing the recombinant protein through a depth filter to provide a filtrate that comprises purified recombinant protein and is substantially free of soluble protein aggregates.

2. The method of claim 1, wherein the capturing is performed using an affinity chromatography resin, an anionic exchange chromatography resin, a cationic exchange chromatography resin, a mixed-mode chromatography resin, a molecular sieve chromatography resin, or a hydrophobic interaction chromatography resin.

3. The method of claim 2, wherein the affinity chromatography resin utilizes a capture mechanism selected from the group consisting of: a protein A-binding capture mechanism, an antibody- or antibody fragment-binding capture mechanism, a substrate-binding capture mechanism, and a cofactor-binding capture mechanism.

4. The method of claim 1, wherein the one or more unit operations in step (b) is selected from the group consisting of:
ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein.

5. (canceled)

6. The method of claim 1, wherein the one or more unit operations in step (b) is polishing using hydrophobic interaction chromatography and ultrafiltration/diafiltration to concentrate the recombinant protein in a solution.

7. (canceled)

8. The method of claim 1, wherein the recombinant protein is flowed through the depth filter in a solution having a pH of between about 4.0 to about 7.5.

9.-10. (canceled)

11. The method of claim 8, wherein the recombinant protein is flowed through the depth filter and both protein aggregates and HCP are reduced by at least 50%.

12. The method of claim 1, wherein the recombinant protein is flowed through the depth filter at a flow rate of between about 25 L/m²/h to about 400 L/m²/h.

13.-15. (canceled)

16. The method of claim 1, wherein the depth filter comprises a filtration medium that is positively charged.

17. (canceled)

18. The method of claim 1, wherein following flowing the recombinant protein through the depth filter, the filtrate is flowed through one or more additional depth filters or a viral filter.

19. The method of claim 1, further comprising prior to step (a):
performing one or more unit operations selected from the group consisting of:
ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein.

20. The method of claim 1, wherein the filtrate comprising purified recombinant protein in step (c) further comprises a reduced level of host cell protein as compared to a level of host cell protein in the recombinant protein that is flowed through the depth filter in step (c).

21. A method of manufacturing a recombinant protein product, the method comprising:
(a) capturing a recombinant protein from a clarified liquid culture medium comprising the recombinant protein;
(b) following capturing, performing one or more unit operations on the solution;
(c) following steps (a) and (b), flowing the recombinant protein through a depth filter to provide a filtrate that comprises purified recombinant protein and is substantially free of soluble protein aggregates; and
(d) performing one or more unit operations on the purified recombinant protein, thereby producing the recombinant protein product.

22.-23. (canceled)

24. The method of claim 21, wherein the one or more unit operations in step (b) is selected from the group consisting of: ultrafiltration/diafiltration to concentrate the recombinant protein in a solution, ion exchange chromatography, hydrophobic interaction chromatography, polishing the recombinant protein, viral inactivation, viral filtration, adjustment of pH, adjustment of ionic strength, and adjustment of both pH and ionic strength of the solution comprising the recombinant protein.

25. (canceled)

26. The method of claim 21, wherein the one or more unit operations in step (b) is polishing using hydrophobic interaction chromatography and ultrafiltration/diafiltration to concentrate the recombinant protein in a solution.

27. The method of claim 21, wherein the recombinant protein is flowed through the depth filter in a solution having a pH of between about 4.0 to about 7.5.

28.-30. (canceled)

31. The method of claim 21, wherein the recombinant protein is flowed through the depth filter at a flow rate of between about 25 L/m²/h to about 400 L/m²/h.

32.-34. (canceled)

35. The method of claim 21, wherein the depth filter comprises a filtration medium that is positively charged.

36. (canceled)

37. The method of claim 21, wherein the one or more unit operations in step (d) is selected from the group consisting of: purifying the recombinant protein, polishing the recombinant protein, inactivating viruses, removing viruses by filtration, adjusting one or both of the pH and ionic concentration of a solution comprising the purified recombinant protein, or passing the fluid through an additional depth filter.

38. The method of claim 37, wherein the one or more unit operations in step (d) is removing viruses by filtration.

39. The method of claim 21, wherein the filtrate comprising purified recombinant protein in step (c) further comprises a reduced level of host cell protein as compared to a level of host cell protein in the recombinant protein that is flowed through the depth filter in step (c).

40. The method of claim 1, wherein the recombinant protein is eculizumab or Alexion 1210.

41.-48. (canceled)
